(19) United States
(12) Patent Application Publication
(10) Pub. No.: US 2026/0117221 A1
(43) Pub. Date: Apr. 30, 2026

(54) METHODS OF PREPARING LIGATION PRODUCT AND SEQUENCING LIBRARY, IDENTIFYING BIOMARKERS, PREDICTING OR DETECTING A DISEASE OR CONDITION

(71) Applicant: Zongli ZHENG, Hong Kong (CN)

(72) Inventors: Zongli ZHENG, Hong Kong (CN); Shifeng LIAN, Stockholm (SE)

(21) Appl. No.: 18/854,057

(22) PCT Filed: Apr. 6, 2023
(86) PCT No.: PCT/CN2023/086601

§ 371 Date: Oct. 3, 2024

Related U.S. Application Data
(60) Provisional application No. 63/362,665, filed on Apr. 8, 2022.

(51) Int. Cl. C12N 15/10; C12Q 1/6886

(52) U.S. Cl. CPC C12N 15/1068; C12N 15/1072; C12Q 1/6886

(57) ABSTRACT

Provided is a method of preparing at least one ligation product from a sample including a plurality of single-strand nucleic acid fragments, the method including the steps of: (a) ligating a first universal oligonucleotide adaptor to at least one single-strand nucleic acid fragment, wherein the first universal oligonucleotide adaptor is configured for ligating to a 3′ end of individual single-strand nucleic acid fragment; and (b) ligating a second universal oligonucleotide adaptor to the at least one single-strand nucleic acid fragment, wherein the second universal oligonucleotide adaptor is configured for ligating to a 5′ end of individual single-strand nucleic acid fragment, thereby at least one ligation product is formed. In another embodiment, provided is at least one cancer biomarker comprising human telomere sequence with two or more consecutive repeats of nucleotide sequence TTAGGG.

### CROSS-REFERENCE TO RELATED APPLICATION

This application claims priority to, and the benefit of, U.S. Provisional Application having Ser. No. 63/362,665 filed on Apr. 8, 2022. The entire contents of the foregoing application are hereby incorporated by reference in its entirety for all purposes.

### REFERENCE TO SEQUENCE LISTING

This application contains a sequence listing which has been submitted electronically in ST.26 (xml) format and is hereby incorporated by reference in its entirety. Said ST.26 copy, created on May 20, 2025, is named “00349155 G024002NPOUS.xml” and is 25 kilobytes in size.

### FIELD OF INVENTION

This invention relates to methods of preparing ligation product and sequencing library from a sample. In some embodiments, the present invention provides methods of identifying biomarkers, and methods of predicting or detecting a disease or condition in a subject.

### BACKGROUND OF INVENTION

Early detection of diseases, especially cancer, is important to allow early intervention in order to improve the chance for successful treatment and survival of the patient. For example, hepatocellular carcinoma (HCC) is the third-most common cause of cancer-related mortality worldwide, which is estimated to cause approximately 830,000 deaths in 2020. The only potential cure for HCC likely is surgery or liver transplantation if the disease is detected early. However, because clinical symptoms associated with the disease are nonspecific, diagnosis is often delayed to advanced stages, when the 5-year survival is less than 18%, whereas early-stage HCC 5-year survival can reach over 50%.

Despite universal hepatitis B virus (HBV) vaccination of newborns and advances in antiviral therapy, chronic HBV infection still affects more than 250 million people, accounting for at least 50% of HCC cases worldwide. International guidelines concordantly recommend HCC screening in patients with HBV infection, with cirrhosis or at high risk for HCC. However, the evidence in supporting the screening is insufficient, due to the lack of highly sensitive surveillance biomarkers for early-stage HCC. For example, the recommended screening strategy—ultrasound and alpha-fetoprotein (AFP) combined—has a sensitivity of 63% for detecting early-stage HCC.

For at least the above reasons, there is a need for novel methods of identifying sensitive biomarkers for early detection of disease or condition.

### SUMMARY OF INVENTION

Disclosed herein are novel methods of preparing at least one ligation product, methods of preparing a sequencing library from a sample including a plurality of single-strand nucleic acid fragments, methods of identifying one or more biomarkers associated with a disease or condition, and methods of making the same.

In some embodiments, provided is a method of preparing at least one ligation product from a sample including a plurality of single-strand nucleic acid fragments, the method including the steps of: (a) ligating a first universal oligonucleotide adaptor to at least one single-strand nucleic acid fragment, wherein the first universal oligonucleotide adaptor is configured for ligating to a 3′ end of individual single-strand nucleic acid fragment; and (b) ligating a second universal oligonucleotide adaptor to the at least one single-strand nucleic acid fragment, wherein the second universal oligonucleotide adaptor is configured for ligating to a 5′ end of individual single-strand nucleic acid fragment, thereby at least one ligation product is formed.

In some embodiments, provided is a method of preparing a sequence library from a sample including a plurality of single-strand nucleic acid fragments, the method including the steps of: (a) ligating a first universal oligonucleotide adaptor to at least one single-strand nucleic acid fragment, wherein the first universal oligonucleotide adaptor is configured for ligating to a 3′ end of individual single-strand nucleic acid fragment; (b) ligating a second universal oligonucleotide adaptor to the at least one single-strand nucleic acid fragment, wherein the second universal oligonucleotide adaptor is configured for ligating to a 5′ end of individual single-strand nucleic acid fragment, thereby at least one ligation product is formed; (c) amplifying the at least one ligation product with a pair of sequencing specific adaptor primers to form a sequencing library, wherein the pair of sequencing specific adaptor primers is at least partially complementary to the first universal oligonucleotide adaptor and the second universal oligonucleotide adaptor respectively.

In some embodiments, provided is a method of identifying one or more biomarkers associated with a disease or condition, including the steps of: (a) obtaining a plurality of samples including a plurality of single-strand nucleic acid fragments from a case group of subjects having the disease or condition and from a control group; (b) for individual sample, ligating a first universal oligonucleotide adaptor to at least one single-strand nucleic acid fragment, wherein the first universal oligonucleotide adaptor is configured for ligating to a 3′ end of individual single-strand nucleic acid fragment; (c) ligating a second universal oligonucleotide adaptor to the at least one single-strand nucleic acid fragment, wherein the second universal oligonucleotide adaptor is configured for ligating to a 5′ end of individual single-strand nucleic acid fragment, thereby at least one ligation product is formed; (d) amplifying the at least one ligation product with a pair of sequencing specific adaptor primers to form individual sequencing library, wherein the pair of sequencing specific adaptor primers is at least partially complementary to the first universal oligonucleotide adaptor and the second universal oligonucleotide adaptor respectively; (e) quantifying and reading the sequencing library to obtain individual sequencing result; and (f) comparing the sequencing results between the case group and the control group, such that one or more biomarkers associated with the disease or condition are identified.

In some embodiments, provided is a method of predicting or detecting a disease or condition in a subject, including the steps of: (a) obtaining a sample including a plurality of single-strand nucleic acid fragments from the subject; (b) ligating a first universal oligonucleotide adaptor to at least one single-strand nucleic acid fragment, wherein the first universal oligonucleotide adaptor is configured for ligating to a 3′ end of individual single-strand nucleic acid fragment; (c) ligating a second universal oligonucleotide adaptor to the at least one single-strand nucleic acid fragment, wherein the second universal oligonucleotide adaptor is configured for ligating to a 5′ end of individual single-strand nucleic acid fragment, thereby at least one ligation product is formed; (d) amplifying the at least one ligation product with a pair of sequencing specific adaptor primers to form a sequencing library, wherein the pair of sequencing specific adaptor primers is at least partially complementary to the first universal oligonucleotide adaptor and the second universal oligonucleotide adaptor respectively; (e) quantifying and reading the sequencing library to obtain a sequencing result of the subject; and (f) analyzing the levels of one or more biomarkers associated with the disease or condition using the sequencing result.

In some embodiments, provided is a method of predicting or detecting cancer in a human subject, including the steps of: (a) obtaining a sample including a plurality of nucleic acid fragments from the subject; and (b) performing a quantitative analysis of the level of at least one biomarker associated with the cancer using the plurality of nucleic acid fragments of the sample, wherein the at least one biomarker includes one or more telomere-containing sequences including at least two consecutive repeats of nucleotide sequence TTAGGG.

Other example embodiments are discussed herein.

There are many advantages of the present application. In some embodiments, the sample comprises a plurality of single-strand nucleic acid fragments. In some embodiments, disclosed herein are novel methods to prepare a sequencing library and uses thereof which is termed as bilateral single-strand sequencing (BLESSING) that allows simple and direct whole genome sequencing library construction, as well as simple and robust analysis of single-stranded DNA. In some embodiments, single-strand library strategy using the novel methods of the present application is able to recover more biological information than the conventional double-strand library strategies. In some embodiments, the novel methods are able to maximally recover circulating cell-free DNA (ccfDNA) including those of ultra-short sizes and to preserve nature DNA fragment ends in biological samples. In some embodiments, the novel methods are able to recognize fragment direction and therefore are able to analyze the sequences by end source (5′ or 3′ of a DNA fragment).

In some embodiments, disclosed herein are novel methods for identifying or screening one or more biomarkers associated with a disease or condition such as cancer using the sequencing results obtained by BLESSING. In some embodiments, the one or more biomarkers identified can be used for accurately predicting or detecting the disease or condition in a given subject.

In certain embodiments, disclosed herein are methods for predicting or detecting a disease or condition in a subject using a sample obtained therefrom, such as a sample comprising circulating cell-free DNA (ccfDNA). In certain embodiments, circulating cell-free DNA (ccfDNA) shed from solid tumors provides a window to detect early cancer in a non-invasive manner. In some embodiments, the novel methods demonstrate high sensitivity in predicting or detecting a disease or condition such as cancer using pre-diagnosis samples. In some embodiments, the novel methods are able to determine if the subject have high or low risk of death. In some embodiments, provided is at least one cancer biomarker comprising human telomere sequence with two or more repeats of nucleotide sequence TTAGGG. In some embodiments, the cancer is hepatocellular carcinoma (HCC) and the biomarkers comprise telomere G-tail (5′-TTAGGG-3′) and ccfDNA end sequences and optionally alpha-fetoprotein (AFP). In some embodiments, the novel methods can be applied for detecting early hepatocellular carcinoma in high-risk populations. In some embodiments, the novel methods can be applied for detecting early cancers of different tissue types, such as kidney cancer, liver cancer, breast cancer, colorectal cancer, pancreatic cancer, uterine cancer, bladder cancer, prostate cancer, lung cancer, testicular cancer, esophageal cancer, head cancer, ovarian cancer, and skin cancer. In some embodiments, as the telomere biology mechanism holds true for all types of cancers, the novel methods can be applied for detecting early cancers of any tissue types.

In certain embodiments, the novel methods include the use of telomeres as biomarkers for predicting or detecting a disease or condition such as cancer. Telomeres, located at the terminal ends of linear chromosome, are closely associated with integrity of the genome, cellular immortalization, and cancer development. These short telomeres capture the characteristics of clone expansion in early-stage cancer, thus can be potentially used as tumor biomarker for early detection.

In certain embodiments, based on hospital HCC cases and a machine learning approach, a model termed “telomere and end sequence phenomenon etymology (Telephone) model” or “Telecon model” was provided for detecting HCC at the initial Discovery phase and then validated in the hepatitis B virus surface antigen (HBsAg)-seropositive cohort. Based on longitudinal samples, an increasing diagnostic performance of Telephone were shown using pre-HCC samples collected at >4 years, 4-3 years, 3-2 years, 2-1 years and 1-0 year before clinical diagnosis of HCC. Telephone showed an estimated positive predict value of 15.2% for HCC diagnosis in one year among a high-risk population and can predict prognosis of HCC cases independent of tumor stage.

In some embodiments, Telephone had a sensitivity of 68.2% (95% CI=52.4-81.4%) in detecting early HCC, yielding an estimated positive predict value of 15.2% among HBV-seropositive population. High Telephone was also associated with poor survival in hospital HCC patients (hazard ratio 3.22, 95% CI=1.49-7.0), independent of tumor stage.

### BRIEF DESCRIPTION OF FIGURES

FIG. 1A shows an example workflow of a method for preparing a ligation product and a sequence library which is termed as bilateral single-strand sequencing (BLESSING) according to an example embodiment.

FIG. 1B is a flowchart of a method of identifying one or more biomarkers associated with a disease or condition according to an example embodiment.

FIG. 1C is a flowchart of a method of predicting or detecting a disease or condition in a subject according to an example embodiment.

FIG. 2A is a diagram which illustrates an example workflow of a study consisted of a population-based cohort for validation (validation phase) and a hospital-based discovery (discovery phase) for initial biomarker identification according to an example embodiment.

FIG. 2B shows size distributions of ccfDNA fragments in discovery and validation phases according to an example embodiment.

FIG. 2C shows definitions of telomere related sequences according to an example embodiment, which can be identified from sequencing data.

FIG. 2D is a schematic diagram which illustrates the extraction of 4 bases at the 5′ end and 3′ end of DNA fragments according to an example embodiment.

FIG. 3A shows case-control comparison of telomere (Telo) and non-telomere (Telo_null) fragments, their reverse complement fragments (TeloRv and TeloRv_null), and fragment end sequences between HCC and non-HCC control groups in terms of p-value versus fold change in the Discovery phase according to an example embodiment.

FIG. 3B shows the results of hierarchical clustering analysis of the same example embodiment of FIG. 3A.

FIG. 3C shows case-control comparison of the proportions of telomere (Telo) and non-telomere (Telo_null) fragments, their reverse complement fragments (TeloRv and TeloRv_null), and fragment end sequences between 1 year Pre-HCC and non-HCC control groups in terms of p-value versus fold change in the Validation phase according to an example embodiment.

FIG. 3D shows the results of hierarchical clustering analysis of the same example embodiment of FIG. 3C.

FIG. 3E shows a graph comparing the example variable importance of Telephone markers and an example equation to calculate a Telephone score to express the contributions of the 4 markers according to an example embodiment.

FIG. 3F shows the distributions of the four Telephone markers, and TeloRv and TeloRv_null by disease status (control, pre-HCC, HCC) and fragment size in Discovery and Validation phases, according to an example embodiment.

FIG. 4A shows comparison of Telephone between controls in discovery phase and independent validation phase with pre-diagnosis samples by cutoff curve analysis, according to an example embodiment.

FIG. 4B shows comparison of AUC of Telephone between controls in discovery phase and independent validation phase with pre-diagnosis samples by ROC curve analysis, according to the same embodiment of FIG. 4A.

FIG. 4C shows comparison of AUC of AFP between controls in discovery phase and independent validation phase with pre-diagnosis samples by ROC curve analysis, according to an example embodiment.

FIG. 4D shows the comparison of sensitivities for detecting HCC using AFP alone, Telephone alone and both (AFP and Telephone), according to an example embodiment.

FIG. 4E shows estimated positive predictive value (PPV) and negative predictive value (NPV), using Telephone alone and both (AFP and Telephone), in a population setting where male chronic HBV carriers have an incidence rate of 525 per 100,000 person-years for HCC (corresponding to the incidence among male HBV-carriers in the entire screening cohort in an example embodiment).

FIG. 4F shows the timeline of pre-HCC blood sample collection in the population cohort, according to an example embodiment. Each line represents one individual. Each dot represents one sampling time point. The statues of Telephone (positive or negative) and AFP (positive or negative) for any blood sample were shown as in the legend.

FIG. 5A shows Kruskal-Wallis tests of Telephone in different BCLC stages, according to an example embodiment.

FIG. 5B shows hazards ratios of patient survival by factors of Telephone, Age, Sex, BCLC and AFP, according to the same embodiment of FIG. 5A.

FIG. 5C shows the survival probability of HCC patients with high or low Telephone over the time, according to the same embodiment of FIG. 5A.

FIG. 5D shows the survival probability of HCC patients with high or low Telephone over the time by different BCLC stages, according to the same example embodiment of FIG. 5A.

FIG. 6A is a schematic diagram showing plasma volumes used in discovery and validation phases, according to the same example embodiment.

FIG. 6B shows total ccfDNA amount of non-HCC and HCC/Pre-HCC in discovery and validation phase, according to an example embodiment.

FIG. 6C shows raw read numbers of sequencing data of non-HCC and HCC/Pre-HCC in discovery and validation phases, according to an example embodiment.

FIGS. 7A and 7B show case-control comparisons of 260 telomere and 4-nt end sequences in discovery phase among 18 strata, namely by fragment size (short/medium/long), end source (5′/3′), and type of end sequence (5p4/3p4/pp4), according to an example embodiment. The darker dots are features with fold change >2 or <0.5.

FIGS. 8 and 9A-9B show the dynamic change of Telephone along the time to diagnosis in 51 HCC patients in whom more than two pre-diagnosis samples were available, according to an example embodiment. In FIGS. 8 and 9A-9B, the dotted line is the Telephone at a cutoff (0.429) with a corresponding specificity at 98%. FIG. 8 shows Telephone changes in a group of pre-HCC patient samples. The solid line shows the Telephone change over time derived by the method of locally estimated scatterplot smoothing. Linear mixed model is used to test the time change trend, and with P<0.001. FIG. 9A-B shows individual Telephone change along the time to diagnosis.

FIGS. 10A and 10B show Telephone distribution by sex, AFP and age in 67 HCC patients in discovery phase (FIG. 10A) and 43 Pre-HCC samples around 1 year before diagnosis in validation phase (FIG. 10B), according to an example embodiment.

FIG. 11A shows motif diversity score (MDS) distribution of Non-HCC and HCC/Pre-HCC in discovery and validation phases, according to an example embodiment. Pre-HCC samples are classified into 5 intervals at >4, 3-4, 2-3, 1-2, and 0-1 year before diagnosis according to the samples collection time. And when more than one sample was evaluated at an interval for one Pre-HCC subject, the mean MDS score is selected.

FIG. 11B shows AUC of ccfDNA motif diversity score (MDS) in discovery and validation phases, according to an example embodiment.

FIG. 11C shows distribution of 6 previous reported end sequence (CCCA, CCAG, CCTG, TAAA, AAAA, TTTT) in discovery and validation phases, according to an example embodiment. Except for non-significant (ns) marked, other groups showed statistically significant difference.

FIG. 11D shows CCCA, CCAG, CCTG, TAAA, AAAA, TTTT end sequence distribution by BCLC stage in the 67 HCC patients from discovery phase, according to an example embodiment.

FIG. 12 shows all AUC values from the 18 analysis strata and by the time before diagnosis in the Validation phase, following LASSO models developed from respective stratum in the Discovery phase, according to an example embodiment.

Patients included in the Discovery phase and Validation phase were mutually exclusive. The 18 strata include stratification by end source (5′/3′), fragment size (short/medium/long), and type of end sequence (5p4/3p4/pp4).

FIG. 13 shows the comparison of library complexity of BLESSING with the Snyder's method, according to an example embodiment.

FIG. 14 shows the principle component analysis of non-HCC controls by experiment batch, according to an example embodiment.

FIG. 15 shows the external evaluation of Telecon score with multiple cancers using data from Snyder et al, according to an example embodiment.

### DETAILED DESCRIPTION

As used herein and in the claims, the terms “comprising” (or any related form such as “comprise” and “comprises”), “including” (or any related forms such as “include” or “includes”), “containing” (or any related forms such as “contain” or “contains”), means including the following elements but not excluding others. It shall be understood that for every embodiment in which the term “comprising” (or any related form such as “comprise” and “comprises”), “including” (or any related forms such as “include” or “includes”), or “containing” (or any related forms such as “contain” or “contains”) is used, this disclosure/application also includes alternate embodiments where the term “comprising”, “including,” or “containing,” is replaced with “consisting essentially of” or “consisting of”. These alternate embodiments that use “consisting of” or “consisting essentially of” are understood to be narrower embodiments of the “comprising”, “including,” or “containing,” embodiments.

For example, alternate embodiments of “a composition comprising A, B, and C” would be “a composition consisting of A, B, and C” and “a composition consisting essentially of A, B, and C.” Even if the latter two embodiments are not explicitly written out, this disclosure/application includes those embodiments. Furthermore, it shall be understood that the scopes of the three embodiments listed above are different.

For the sake of clarity, “comprising”, including, and “containing”, and any related forms are open-ended terms which allows for additional elements or features beyond the named essential elements, whereas “consisting of” is a closed end term that is limited to the elements recited in the claim and excludes any element, step, or ingredient not specified in the claim.

As used herein, the singular forms “a”, “an” and “the” are intended to include the plural forms as well, unless the context clearly indicates otherwise. Where a range is referred in the specification, the range is understood to include each discrete point within the range. For example, 1-7 means 1, 2, 3, 4, 5, 6, and 7.

As used herein and in the claims, a “subject” refers to animals such as mammals, including, but not limited to, primates (e.g., humans), cows, sheep, goats, horses, dogs, cats, rabbits, rats, mice and the like.

As used herein and in the claims, “enriching” means increasing the proportion of molecule target of interest among all molecules from a sample.

As used herein and in the claims, “nucleic acid fragments” means the nucleic acid has been fragmented into shorter pieces. In certain embodiments, the nucleic acid fragmented into typical sizes peaking at around 12 to 19 nucleotides (nt), 20 to 60 nt, 61 to 100 nt, 101 to 300 nt, 301 to 500 nt, and/or 501 to 1000 nt.

As used herein and in the claims “high molecular weight DNA” refers to DNA that has not been fragmented into shorter pieces. In certain embodiments, a high molecular weight DNA can be around 300 bp or longer. In certain embodiments, a high molecular weight DNA can be around 500 bp or longer. In certain embodiments, a high molecular weight DNA is derived from genomic DNA.

As used herein and in the claims, “BLESSING (bilateral jingle-strand sequencing” is a technique for preparing sequencing library as described in the present disclosure. In some embodiments, BLESSING allows for construction of whole genome, single stranded sequencing library. In some embodiments, BLESSING is able to sequence short DNA fragments, such as circulating cell-free DNA (ccfDNA).

As used herein and in the claims, “Telephone (telomere and end sequence phenomenon etymology)” or “Telecon” is a biomarker model for prediction or detection of a disease or disorder. In some embodiments, Telephone or Telecon is formulated by a logistic regression model for early detection or prediction for hepatocellular carcinoma (HCC).

As used herein and in the claims, “telomere” refers to a region of repetitive nucleotide sequences located at the terminal ends of linear chromosome.

As used herein and in the claims, “telomere-related sequences” refers to sequences in a sequencing library that are screened for the occurrence of telomere, including telomere-containing sequences and non-telomere containing sequences. For example, for a human sample, human telomere contains the characteristic sequence 5′-TTAGGG-3′, and telomere-related sequence refers to telomere-containing sequences with at least two consecutive telomere repeats 5′-TTAGGGTTAGGG-3′ (SEQ ID NO: 5), and non-telomere containing sequences do not contain 5′-TTAGGG-3′.

As used herein and in the claims, “fragment end sequences” refers to nucleotide sequences that located at the 5′ or 3′ ends of DNA fragments. In some embodiments, fragment end sequences include 4-base DNA fragment end sequences at 3′ end (3p4), at 5′ end (5p4), and 2 genome-inferred bases plus 2 sequenced fragment-end bases in the 5′ to 3′ direction (pp4).

As used herein and in the claims, “universal oligonucleotide adaptor” refers to a nucleic acid molecule comprised of two strands (a top strand and a bottom strand) and comprising a first ligatable 5′ protrude end and a second un-ligatable end. In some embodiments, the top strand of the universal oligonucleotide adaptor comprises a 5′ duplex portion, and the bottom strand comprises an unpaired 5′ portion, a 3′ duplex portion, and nucleic acid sequences identical to a first and second sequencing primers. The duplex portions of the adaptor may be substantially complementary and the duplex portion is of sufficient length to remain in duplex form at the ligation temperature. In certain embodiments, the top and/or bottom strands of the first and/or second universal oligonucleotide adaptors comprise a 3′ blocking group, such as an inverted T nucleotide or a phosphorylation. In certain embodiments, the top strand and the bottom strand are connected to each other and form a hairpin loop. The term “sufficient” means that the number of bases in the duplex portion is long enough so that the bonding therebetween can keep in duplex form at the ligation temperature.

As used herein and in the claims, “a universal oligonucleotide adaptor primer” refers to a primer that can anneal to part of the sequence of the universal oligonucleotide adaptor.

Although the description referred to particular embodiments, the disclosure should not be construed as limited to the embodiments set forth herein.

### NUMBERED EMBODIMENTS

### Set 1

Embodiment 1. A method of preparing nucleic acid from a sample comprising a plurality of single-strand nucleic acid fragments, the method comprising the steps of: (a) ligating a first universal oligonucleotide adaptor to the sample to produce a ligation product, wherein the universal oligonucleotide adaptor is configured for ligating to a 3′ end of the single-strand nucleic acid fragments; and (b) ligating a second universal oligonucleotide adaptor to the above sample to produce a ligation product, wherein the second universal oligonucleotide adaptor is configured for ligating to a 5′ end of the single-strand nucleic acid fragments.

Embodiment 2. The method of embodiment 1, wherein prior to the step (a), the method further comprises the steps of: (i) dephosphorylating a 5′ end of the single-strand nucleic acid fragments; and prior to step (b), the method further comprises the step of: (ii) phosphorylating a 5′ end of the single-strand nucleic acid fragments.

Embodiment 3. The method of embodiment 1, wherein the first universal oligonucleotide adaptor comprises: a 5′ recessive end, the 5′ recessive end is configured for ligating to the 3′ end of the single-strand nucleic acid fragments; and a duplex portion of the universal oligonucleotide adaptor is of sufficient length to remain in duplex form in the step (a).

Embodiment 4. The method of embodiment 1, wherein the second universal oligonucleotide adaptor comprises: a 3′ recessive end, the 3′ recessive end is configured for ligating to the 5′ end of the single-strand nucleic acid fragments; and a duplex portion of the universal oligonucleotide adaptor is of sufficient length to remain in duplex form in the step (b).

Embodiment 5. The method of any one of the preceding embodiments, wherein the universal oligonucleotide adaptor comprises a hairpin loop connecting a portion of the duplex form.

Embodiment 6. The method of any one of the preceding embodiments, wherein the universal oligonucleotide adaptor comprises a single strand segment comprising three to twenty random nucleotides as a unique molecular index (UMI) for tracing individual original molecules.

Embodiment 7. The method of any one of the preceding embodiments, wherein the step (b) further comprises the step of forming a sequencing library by amplification using a pair of sequencing specific adaptor primers.

Embodiment 8. The method of any one of the preceding embodiments, wherein after the step (b), the method further comprises enrichment of at least one targeted nucleic acid from step (b), using at least one targeted specific primer and one of the adaptor primers.

Embodiment 9. The method of embodiment 1, wherein after the step (b), further comprises the step of: (i) sequencing the sequencing library using a sequencing primer pair, wherein the sequencing primer pair is at least partially complementary to opposite strands of the ligation product in (b), respectively.

Embodiment 10. The method of any one of the preceding embodiments, wherein the sample comprises a plurality of DNA fragments prepared from high molecular weight DNA of longer than 500 basepairs (e.g., genomic DNA).

Embodiment 11. The method of any one of the preceding embodiments, wherein the plurality of single-strand nucleic acid fragments is prepared from denaturation of double-strand DNA fragments.

Embodiment 12. The method of any one of the preceding embodiments, wherein the sample comprises single-strand cDNA fragments prepared from reverse transcription of RNA fragments.

Embodiment 13. The method of any one of the preceding embodiments, wherein the method further comprises the step of analyzing the plurality of nucleic acids fragments.

Embodiment 14. The method of any one of the preceding embodiments, wherein the sample is from a mammal (e.g., a human).

Embodiment 15. The method of embodiment 14, wherein the human is an individual known to have or suspected of having a disease (e.g. a cancer or a genetic disorder).

Embodiment 16. The method of embodiment 15, wherein one or more of the target sequence comprise one or more markers for the cancer.

Embodiment 17. The method of embodiment 16, wherein the human is a fetus.

Embodiment 18. The method of any one of embodiments 1-19, wherein the sample is from a blood sample.

Embodiment 19. The method of any one of embodiments 1-19, wherein the sample is cell-free nucleic acids extracted from a blood sample.

Embodiment 20. The method of any one of embodiments 1-19, wherein the sample is nucleic acids extracted from lymphocytes in a blood sample for T-cell and B-cell receptor profiling.

Embodiment 21. The method of any one of embodiments 1-19, wherein the sample is nucleic acids extracted from circulating tumor cells.

Embodiment 22. The method of any one of preceding embodiments, wherein the target sequence contains two consecutive telomere sequences (e.g. TTAGGGTTAGGG (SEQ ID NO: 5) in human samples).

### Set 2

Embodiment 1. A method of preparing at least one ligation product from a sample comprising a plurality of single-strand nucleic acid fragments, the method comprising the steps of: (a) ligating a first universal oligonucleotide adaptor to at least one single-strand nucleic acid fragment, wherein the first universal oligonucleotide adaptor is configured for ligating to a 3′ end of individual single-strand nucleic acid fragment; and (b) ligating a second universal oligonucleotide adaptor to the at least one single-strand nucleic acid fragment, wherein the second universal oligonucleotide adaptor is configured for ligating to a 5′ end of individual single-strand nucleic acid fragment, thereby at least one ligation product is formed.

Embodiment 2. The method of embodiment 1, wherein prior to the step (a), the method further comprises the step of: dephosphorylating the 5′ end of the at least one single-strand nucleic acid fragment.

Embodiment 3. The method of embodiment 1 or 2, wherein prior to the step (b), the method further comprises the step of: phosphorylating the 5′ end of the at least one single-strand nucleic acid fragment.

Embodiment 4. The method of any one of the preceding embodiments, wherein the first universal oligonucleotide adaptor further comprises: a top strand having a 5′ recessive end, wherein the 5′ recessive end is configured for ligating to the 3′ end of the individual single-strand nucleic acid fragment; and a bottom strand partially complementary to the top strand to form a duplex portion, wherein the duplex portion of the first universal oligonucleotide adaptor is of sufficient length to remain in duplex form in the step (a).

Embodiment 5. The method of embodiment 4, wherein the bottom strand of the first universal oligonucleotide adaptor comprises an unpaired 3′ portion.

Embodiment 6. The method of any one of the preceding embodiments, wherein the second universal oligonucleotide adaptor further comprises: a top strand having a 3′ recessive end, wherein the 3′ recessive end is configured for ligating to the 5′ end of the individual single-strand nucleic acid fragment; and a bottom strand partially complementary to the top strand to form a duplex portion, wherein the duplex portion of the second universal oligonucleotide adaptor is of sufficient length to remain in duplex form in the step (b).

Embodiment 7. The method of embodiment 6, wherein the bottom strand of the second universal oligonucleotide adaptor comprises an unpaired 5′ portion.

Embodiment 8. The method of any one of the preceding embodiments, wherein the first universal oligonucleotide adaptor and/or the second universal oligonucleotide adaptor comprise a hairpin loop connecting a portion of the duplex form.

Embodiment 9. The method of any one of the preceding embodiments, wherein the first universal oligonucleotide adaptor and/or the second universal oligonucleotide adaptor comprise a single strand segment comprising three to twenty random nucleotides as a unique molecular index (UMI) for tracing individual original molecules.

Embodiment 10. The method of any one of embodiments 4-9, wherein the bottom strand of the first universal oligonucleotide adaptor comprises a nucleotide sequence of SEQ ID NO:3, and the top strand of the first universal oligonucleotide adaptor comprises a nucleotide sequence of SEQ ID NO:4.

Embodiment 11. The method of any one of embodiments 6-10, wherein the bottom strand of the second universal oligonucleotide adaptor comprises nucleotide sequence SEQ ID NO:1, and the top strand of the second universal oligonucleotide adaptor comprises a nucleotide sequence of SEQ ID NO:2.

Embodiment 12. The method of any one of the preceding embodiments, further comprises the step of: amplifying the at least one ligation product with a pair of sequencing specific adaptor primers to form a sequencing library, wherein the pair of sequencing specific adaptor primers is at least partially complementary to the first universal oligonucleotide adaptor and the second universal oligonucleotide adaptor, respectively.

Embodiment 13. The method of embodiment 12, wherein the method further comprises the step of sequencing the sequencing library using a sequencing primer pair.

Embodiment 14. The method of any one of the preceding embodiments, further comprises the step of: enriching at least one targeted nucleic acid from the at least one ligation product, using at least one target specific primer and at least one universal oligonucleotide adaptor primer that is at least partially complementary to the first or second universal oligonucleotide adaptor.

Embodiment 15. The method of any one of the preceding embodiments, wherein the sample comprises a plurality of DNA fragments prepared from high molecular weight DNA.

Embodiment 16. The method of any one of the preceding embodiments, wherein the plurality of single-strand nucleic acid fragments is prepared from denaturation of double-strand DNA fragments.

Embodiment 17. The method of any one of the preceding embodiments, wherein the sample comprises single-strand cDNA fragments prepared from reverse transcription of RNA fragments.

Embodiment 18. The method of any one of the preceding embodiments, wherein the sample is from human.

Embodiment 19. The method of any one of the preceding embodiments, wherein the sample is derived from a blood sample.

Embodiment 20. The method of any one of the preceding embodiments, wherein the sample is cell-free nucleic acids extracted from a blood sample.

Embodiment 21. The method of any one of the preceding embodiments, wherein the sample is nucleic acids extracted from lymphocytes in a blood sample for T-cell and B-cell receptor profiling.

Embodiment 22. The method of any one of the preceding embodiments, wherein the sample is nucleic acids extracted from circulating tumor cells.

Embodiment 23. A method of preparing a sequence library from a sample comprising a plurality of single-strand nucleic acid fragments, the method comprising the steps of: (a) ligating a first universal oligonucleotide adaptor to at least one single-strand nucleic acid fragment, wherein the first universal oligonucleotide adaptor is configured for ligating to a 3′ end of individual single-strand nucleic acid fragment; (b) ligating a second universal oligonucleotide adaptor to the at least one single-strand nucleic acid fragment, wherein the second universal oligonucleotide adaptor is configured for ligating to a 5′ end of individual single-strand nucleic acid fragment, thereby at least one ligation product is formed; (c) amplifying the at least one ligation product with a pair of sequencing specific adaptor primers to form a sequencing library, wherein the pair of sequencing specific adaptor primers is at least partially complementary to the first universal oligonucleotide adaptor and the second universal oligonucleotide adaptor respectively.

Embodiment 24. The method of embodiment 23, further comprises the step of: (d) sequencing the sequencing library using a sequencing primer pair.

Embodiment 25. The method of embodiment 23 or 24, wherein prior to the step (a), the method further comprises the step of: dephosphorylating the 5′ end of the at least one single-strand nucleic acid fragment.

Embodiment 26. The method of any one of embodiments 23 to 26, wherein prior to the step (b), the method further comprises the step of: phosphorylating the 5′ end of the at least one single-strand nucleic acid fragment.

Embodiment 27. The method of any one of the preceding embodiments, wherein the first universal oligonucleotide adaptor further comprises: a top strand with a 5′ recessive end, wherein the 5′ recessive end is configured for ligating to the 3′ end of the individual single-strand nucleic acid fragment; and a bottom strand partially complementary to the top strand to form a duplex portion, wherein the duplex portion of the first universal oligonucleotide adaptor is of sufficient length to remain in duplex form in the step (a).

Embodiment 28. The method of embodiment 27, wherein the bottom strand of the first universal oligonucleotide adaptor comprises an unpaired 3′ portion.

Embodiment 29. The method of any one of the preceding embodiments, wherein the second universal oligonucleotide adaptor further comprises: a top strand with a 3′ recessive end, wherein the 3′ recessive end is configured for ligating to the 5′ end of the individual single-strand nucleic acid fragment; and a bottom strand partially complementary to the top strand to form a duplex portion, wherein the duplex portion of the second universal oligonucleotide adaptor is of sufficient length to remain in duplex form in the step (b).

Embodiment 30. The method of embodiment 29, wherein the bottom strand of the second universal oligonucleotide adaptor comprises an unpaired 5′ portion.

Embodiment 31. The method of any one of the preceding embodiments, wherein the first universal oligonucleotide adaptor and/or the second universal oligonucleotide adaptor comprise a hairpin loop connecting a portion of the duplex form.

Embodiment 32. The method of any one of the preceding embodiments, wherein the first universal oligonucleotide adaptor and/or the second universal oligonucleotide adaptor comprise three to twenty random nucleotides as a unique molecular index (UMI) for tracing individual original molecules.

Embodiment 33. The method of any one of embodiments 27-32, wherein the bottom strand of the first universal oligonucleotide adaptor comprises a nucleotide sequence of SEQ ID NO:3, and the top strand of the first universal oligonucleotide adaptor comprises a nucleotide sequence of SEQ ID NO:4.

Embodiment 34. The method of any one of embodiments 29-33, wherein the bottom strand of the second universal oligonucleotide adaptor comprises a sequence of SEQ ID NO:1, and the top strand of the second universal oligonucleotide adaptor comprises a sequence of SEQ ID NO:2.

Embodiment 35. The method of any one of the preceding embodiments, wherein after the step (b), the method further comprises the step of: enriching at least one targeted nucleic acid from the at least one ligation product, using at least one target specific primer and at least one universal oligonucleotide adaptor primer that is at least partially complementary to the first or second universal oligonucleotide adaptor.

Embodiment 36. The method of any one of the preceding embodiments, wherein the sample comprises a plurality of DNA fragments prepared from high molecular weight DNA.

Embodiment 37. The method of any one of the preceding embodiments, wherein the plurality of single-strand nucleic acid fragments is prepared from denaturation of double-strand DNA fragments.

Embodiment 38. The method of any one of the preceding embodiments, wherein the sample comprises single-strand cDNA fragments prepared from reverse transcription of RNA fragments.

Embodiment 39. The method of any one of the preceding embodiments, wherein the sample is from human.

Embodiment 40. The method of any one of the preceding embodiments, wherein the sample is derived from a blood sample.

Embodiment 41. The method of any one of the preceding embodiments, wherein the sample is cell-free nucleic acids extracted from a blood sample.

Embodiment 42. The method of any one of the preceding embodiments, wherein the sample is nucleic acids extracted from lymphocytes in a blood sample for T-cell and B-cell receptor profiling.

Embodiment 43. The method of any one of the preceding embodiments, wherein the sample is nucleic acids extracted from circulating tumor cells.

Embodiment 44. A method of identifying one or more biomarkers associated with a disease or condition, comprising the steps of: (a) obtaining a plurality of samples comprising a plurality of single-strand nucleic acid fragments from a case group of subjects having the disease or condition and from a control group; (b) for individual sample, ligating a first universal oligonucleotide adaptor to at least one single-strand nucleic acid fragment, wherein the first universal oligonucleotide adaptor is configured for ligating to a 3′ end of individual single-strand nucleic acid fragment; (c) ligating a second universal oligonucleotide adaptor to the at least one single-strand nucleic acid fragment, wherein the second universal oligonucleotide adaptor is configured for ligating to a 5′ end of individual single-strand nucleic acid fragment, thereby at least one ligation product is formed; (d) amplifying the at least one ligation product with a pair of sequencing specific adaptor primers to form individual sequencing library, wherein the pair of sequencing specific adaptor primers is at least partially complementary to the first universal oligonucleotide adaptor and the second universal oligonucleotide adaptor respectively; (e) quantifying and reading the sequencing library to obtain individual sequencing result; and (f) comparing the sequencing results between the case group and the control group, such that one or more biomarkers associated with the disease or condition are identified.

Embodiment 45. The method of embodiment 44, wherein the step (f) further comprises the step of: (i) comparing proportions of individual biomarker between the case group and the control group using Wilcoxon rank-sum test; (ii) identifying individual biomarker with fold-difference of the proportions that is greater or equal to 2, or lesser or equal to 0.5.

Embodiment 46. The method of embodiment 44 or 45, wherein the step (f) further comprises the steps of: (i) evaluating individual identified biomarker using logistic regression model with a Least Absolute Shrinkage and Selection Operator (LASSO) penalty to obtain a LASSO coefficient; and (ii) selecting one or more biomarkers with a non-zero LASSO coefficient among the identified biomarkers.

Embodiment 47. The method of embodiment 46, wherein the step (f) further comprises the steps of: (iii) formulating a logistic regression model using the LASSO coefficient based on the selected one or more biomarkers, such that a Telomere and end sequence phenomenon etymology (Telephone) score is obtained.

Embodiment 48. The method of embodiment 47, further comprising the step of: (iv) validating the logistic regression model in a prospective cohort of subjects to determine the performance of the logistic regression model in detecting the disease or condition.

Embodiment 49. The method of any one of embodiments 44-48, wherein the subjects are human.

Embodiment 50. The method of any one of the preceding embodiments, wherein the disease or condition is cancer or autoimmune disease.

Embodiment 51. The method of embodiment 50, wherein the cancer is selected from a group consisting of kidney cancer, liver cancer, breast cancer, colorectal cancer, pancreatic cancer, uterine cancer, bladder cancer, prostate cancer, lung cancer, testicular cancer, esophageal cancer, head cancer, ovarian cancer, and skin cancer.

Embodiment 52. The method of embodiment 50, wherein the cancer is hepatocellular carcinoma (HCC).

Embodiment 53. The method of any one of the preceding embodiments, wherein the one or more biomarkers comprise one or more telomere-related sequences and/or one or more fragment end sequences.

Embodiment 54. The method of embodiment 53, wherein the one or more telomere-related sequences comprise: (i) one or more telomere-containing sequences comprising at least two consecutive repeats of nucleotide sequence TTAGGG; and (ii) one or more non-telomere containing sequences that do not comprise nucleotide sequence TTAGGG;

Embodiment 55. The method of embodiment 53 or 54, wherein the one or more fragment end sequences comprise nucleotide sequences CAAA and/or GATG.

Embodiment 56. The method of any one of the preceding embodiments, wherein prior to the step (b), the method further comprises the step of: dephosphorylating the 5′ end of the at least one single-strand nucleic acid fragment.

Embodiment 57. The method of any one of the preceding embodiments, wherein prior to the step (c), the method further comprises the step of: phosphorylating the 5′ end of the at least one single-strand nucleic acid fragment.

Embodiment 58. The method of any one of the preceding embodiments, wherein the first universal oligonucleotide adaptor further comprises: a top strand having a 5′ recessive end, wherein the 5′ recessive end is configured for ligating to the 3′ end of the individual single-strand nucleic acid fragment; and a bottom strand partially complementary to the top strand to form a duplex portion, wherein the duplex portion of the first universal oligonucleotide adaptor is of sufficient length to remain in duplex form in the step (b).

Embodiment 59. The method of embodiment 58, wherein the bottom strand of the first universal oligonucleotide adaptor comprises an unpaired 3′ portion.

Embodiment 60. The method of any one of the preceding embodiments, wherein the second universal oligonucleotide adaptor further comprises: a top strand having a 3′ recessive end, wherein the 3′ recessive end is configured for ligating to the 5′ end of the individual single-strand nucleic acid fragment; and a bottom strand partially complementary to the top strand to form a duplex portion, wherein the duplex portion of the second universal oligonucleotide adaptor is of sufficient length to remain in duplex form in the step (c).

Embodiment 61. The method of embodiment 60, wherein the bottom strand of the second universal oligonucleotide adaptor comprises an unpaired 5′ portion.

Embodiment 62. The method of any one of the preceding embodiments, wherein the first universal oligonucleotide adaptor and/or the second universal oligonucleotide adaptor comprise a hairpin loop connecting a portion of the duplex form.

Embodiment 63. The method of any one of the preceding embodiments, wherein the first universal oligonucleotide adaptor and/or the second universal oligonucleotide adaptor comprise three to twenty random nucleotides as a unique molecular index (UMI) for tracing individual original molecules.

Embodiment 64. The method of any one of embodiments 58-63, wherein the bottom strand of the first universal oligonucleotide adaptor comprises a nucleotide sequence of SEQ ID NO:3, and the top strand of the first universal oligonucleotide adaptor comprises a nucleotide sequence of SEQ ID NO:4.

Embodiment 65. The method of any one of embodiments 60-64, wherein the bottom strand of the second universal oligonucleotide adaptor comprises nucleotide sequence SEQ ID NO:1, and the top strand of the second universal oligonucleotide adaptor comprises a nucleotide sequence of SEQ ID NO:2.

Embodiment 66. The method of any one of the preceding embodiments, wherein the sample comprises a plurality of DNA fragments prepared from high molecular weight DNA.

Embodiment 67. The method of any one of the preceding embodiments, wherein the plurality of single-strand nucleic acid fragments is prepared from denaturation of double-strand DNA fragments.

Embodiment 68. The method of any one of the preceding embodiments, wherein the sample comprises single-strand cDNA fragments prepared from reverse transcription of RNA fragments.

Embodiment 69. The method of any one of the preceding embodiments, wherein the sample is from a blood sample.

Embodiment 70. The method of any one of the preceding embodiments, wherein the sample is cell-free nucleic acids extracted from a blood sample.

Embodiment 71. The method of any one of the preceding embodiments, wherein the sample is nucleic acids extracted from lymphocytes in a blood sample for T-cell and B-cell receptor profiling.

Embodiment 72. The method of any one of the preceding embodiments, wherein the sample is nucleic acids extracted from circulating tumor cells.

Embodiment 73. A method of predicting or detecting a disease or condition in a subject, comprising the steps of: (a) obtaining a sample comprising a plurality of single-strand nucleic acid fragments from the subject; (b) ligating a first universal oligonucleotide adaptor to at least one single-strand nucleic acid fragment, wherein the first universal oligonucleotide adaptor is configured for ligating to a 3′ end of individual single-strand nucleic acid fragment; (c) ligating a second universal oligonucleotide adaptor to the at least one single-strand nucleic acid fragment, wherein the second universal oligonucleotide adaptor is configured for ligating to a 5′ end of individual single-strand nucleic acid fragment, thereby at least one ligation product is formed; (d) amplifying the at least one ligation product with a pair of sequencing specific adaptor primers to form a sequencing library, wherein the pair of sequencing specific adaptor primers is at least partially complementary to the first universal oligonucleotide adaptor and the second universal oligonucleotide adaptor respectively; (e) quantifying and reading the sequencing library to obtain a sequencing result of the subject; and (f) analyzing the levels of one or more biomarkers associated with the disease or condition using the sequencing result.

Embodiment 74. The method of embodiment 73, wherein the one or more biomarkers associated with the disease or condition are identified by the method of any one of claims 46-72.

Embodiment 75. The method of embodiment 73 or 74, wherein the subject is human.

Embodiment 76. The method of any one of the preceding embodiments, wherein the disease or condition is cancer or autoimmune disease.

Embodiment 77. The method of embodiment 76, wherein the cancer is selected from a group consisting of kidney cancer, liver cancer, breast cancer, colorectal cancer, pancreatic cancer, uterine cancer, bladder cancer, prostate cancer, lung cancer, testicular cancer, esophageal cancer, head cancer, ovarian cancer, and skin cancer.

Embodiment 78. The method of embodiment 76, wherein the cancer is hepatocellular carcinoma (HCC).

Embodiment 79. The method of any one of the preceding embodiments, wherein the one or more biomarkers comprise one or more telomere-related sequences and/or one or more fragment end sequences.

Embodiment 80. The method of embodiment 79, wherein the one or more telomere-related sequences comprise: (i) one or more telomere-containing sequences comprising at least two consecutive repeats of nucleotide sequence TTAGGG; and (ii) one or more non-telomere containing sequences that do not comprise nucleotide sequence TTAGGG.

Embodiment 81. The method of embodiment 79 or 80, wherein the one or more fragment end sequences comprise nucleotide sequences CAAA and/or GATG.

Embodiment 82. The method of any one of embodiments 79-81, wherein the disease or condition is hepatocellular carcinoma (HCC), wherein the step (f) comprises the steps of: (i) determining a Telomere and end sequence phenomenon etymology (Telephone) score using the sequencing result with the following formula:

$$ln ( \frac{Telephone}{1 - Telephone} ) = 302 + 3320 \times Telo - 610 \times Telo\_null + 356 \times CAAA + 32 \times GATG$$

wherein Telephone refers to the Telephone score, Telo is a level of one or more telomere-containing sequences comprising at least two consecutive repeats of nucleotide sequence TTAGGG, Telo_null is a level of one or more non-telomere containing sequences that do not comprise nucleotide sequence TTAGGG, CAAA is a level of one or more fragment end sequences comprising nucleotide sequence CAAA, and GATC is a level of one or more fragment end sequences comprising nucleotide sequence GATG; (ii) determining the subject as having a high risk for HCC if the Telephone score is above 0.429.

Embodiment 83. The method of embodiment 82, wherein the step (f) further comprises the step of: (iii) determining the subject as having a high risk of death if the Telephone score is above 0.868, and (iv) determining the subject as having a low risk of death if the Telephone score is below or equal to 0.868.

Embodiment 84. The method of embodiments 82 or 83, further comprising the steps of: (i) determining a serum level of alpha-fetoprotein (AFP) in the subject; and (ii) determining the subject as having a high risk for HCC if the serum level of AFP is above 20 ng/mL and the Telephone score is above 0.429.

Embodiment 85. The method of any one of the preceding embodiments, wherein prior to the step (b), the method further comprises the step of: dephosphorylating the 5′ end of the at least one single-strand nucleic acid fragment.

Embodiment 86. The method of any one of the preceding embodiments, wherein prior to the step (c), the method further comprises the step of: phosphorylating the 5′ end of the at least one single-strand nucleic acid fragment.

Embodiment 87. The method of any one of the preceding embodiments, wherein the first universal oligonucleotide adaptor further comprises: a top strand having a 5′ recessive end, wherein the 5′ recessive end is configured for ligating to the 3′ end of the individual single-strand nucleic acid fragment; and a bottom strand partially complementary to the top strand to form a duplex portion, wherein the duplex portion of the first universal oligonucleotide adaptor is of sufficient length to remain in duplex form in the step (b).

Embodiment 88. The method of embodiment 87, wherein the bottom strand of the first universal oligonucleotide adaptor comprises an unpaired 3′ portion.

Embodiment 89. The method of any one of the preceding embodiments, wherein the second universal oligonucleotide adaptor further comprises: a top strand having a 3′ recessive end, wherein the 3′ recessive end is configured for ligating to the 5′ end of the individual single-strand nucleic acid fragment; and a bottom strand partially complementary to the top strand to form a duplex portion, wherein the duplex portion of the second universal oligonucleotide adaptor is of sufficient length to remain in duplex form in the step (c).

Embodiment 90. The method of embodiment 89, wherein the bottom strand of the second universal oligonucleotide adaptor comprises an unpaired 5′ portion.

Embodiment 91. The method of any one of the preceding embodiments, wherein the first universal oligonucleotide adaptor and/or the second universal oligonucleotide adaptor comprise a hairpin loop connecting a portion of the duplex form.

Embodiment 92. The method of any one of the preceding embodiments, wherein the first universal oligonucleotide adaptor and/or the second universal oligonucleotide adaptor comprise three to twenty random nucleotides as a unique molecular index (UMI) for tracing individual original molecules.

Embodiment 93. The method of any one of embodiments 87-92, wherein the bottom strand of the first universal oligonucleotide adaptor comprises a nucleotide sequence of SEQ ID NO:3, and the top strand of the first universal oligonucleotide adaptor comprises a nucleotide sequence of SEQ ID NO:4.

Embodiment 94. The method of any one of embodiments 89-93, wherein the bottom strand of the second universal oligonucleotide adaptor comprises nucleotide sequence SEQ ID NO:1, and the top strand of the second universal oligonucleotide adaptor comprises a nucleotide sequence of SEQ ID NO:2.

Embodiment 95. The method of any one of the preceding embodiments, wherein the sample comprises a plurality of DNA fragments prepared from high molecular weight DNA.

Embodiment 96. The method of any one of the preceding embodiments, wherein the plurality of single-strand nucleic acid fragments is prepared from denaturation of double-strand DNA fragments.

Embodiment 97. The method of any one of the preceding embodiments, wherein the sample comprises single-strand cDNA fragments prepared from reverse transcription of RNA fragments.

Embodiment 98. The method of any one of the preceding embodiments, wherein the sample is from a blood sample.

Embodiment 99. The method of any one of the preceding embodiments, wherein the sample is cell-free nucleic acids extracted from a blood sample.

Embodiment 100. The method of any one of the preceding embodiments, wherein the sample is nucleic acids extracted from lymphocytes in a blood sample for T-cell and B-cell receptor profiling.

Embodiment 101. The method of any one of the preceding embodiments, wherein the sample is nucleic acids extracted from circulating tumor cells.

Embodiment 102. A method of predicting or detecting cancer in a human subject, comprising the steps of: (a) obtaining a sample comprising a plurality of nucleic acid fragments from the subject; and (b) performing a quantitative analysis of the level of at least one biomarker associated with the cancer using the plurality of nucleic acid fragments of the sample, wherein the at least one biomarker comprises one or more telomere-containing sequences comprising at least two consecutive repeats of nucleotide sequence TTAGGG.

Embodiment 103. The method of embodiment 102, wherein the one or more telomere-containing sequences do not comprise a single set of nucleotide sequence TTAGGG with no consecutive repeats.

Embodiment 104. The method of embodiment 102 or 103, wherein the quantitative analysis is performed by quantitative real-time PCR (qPCR) or digital PCR (dPCR).

Embodiment 105. The method of embodiment 104, wherein the quantitative real-time PCR or digital PCR (dPCR) is performed by using a target-specific primer pair, wherein at least one primer in the target-specific primer pair is at least partially complementary to the at least one biomarker.

Embodiment 106. The method of any one of the preceding embodiments, wherein the cancer is selected from a group consisting of kidney cancer, liver cancer, breast cancer, colorectal cancer, pancreatic cancer, uterine cancer, bladder cancer, prostate cancer, lung cancer, testicular cancer, esophageal cancer, head cancer, ovarian cancer, and skin cancer.

Embodiment 107. The method of any one of the preceding embodiments, wherein the cancer is hepatocellular carcinoma (HCC).

Embodiment 108. The method of any one of the preceding embodiments, wherein the plurality of nucleic acid fragments is prepared by fragmentizing and/or denaturing high molecular weight DNA.

Embodiment 109. The method of any one of the preceding embodiments, wherein the plurality of nucleic acid fragments comprise single-strand cDNA fragments prepared from reverse transcription of RNA fragments.

Embodiment 110. The method of any one of the preceding embodiments, wherein the sample is prepared by extracting a blood sample of the subject.

Embodiment 111. The method of any one of the preceding embodiments, wherein the sample is prepared by isolating cell-free nucleic acids extracted from a blood sample of the subject.

Embodiment 112. The method of any one of the preceding embodiments, wherein the sample is prepared by isolating nucleic acids extracted from lymphocytes in a blood sample of the subject for T-cell and B-cell receptor profiling.

Embodiment 113. The method of any one of the preceding embodiments, wherein the sample is prepared by isolating nucleic acids extracted from circulating tumor cells.

### EXAMPLES

Provided herein are examples that describe in more detail certain embodiments of the present disclosure. The examples provided herein are merely for illustrative purposes and are not meant to limit the scope of the invention in any way. All references given below and elsewhere in the present application are hereby included by reference.

### Example 1: Example Workflow of a Method for Preparing a Ligation Product and a Sequence Library

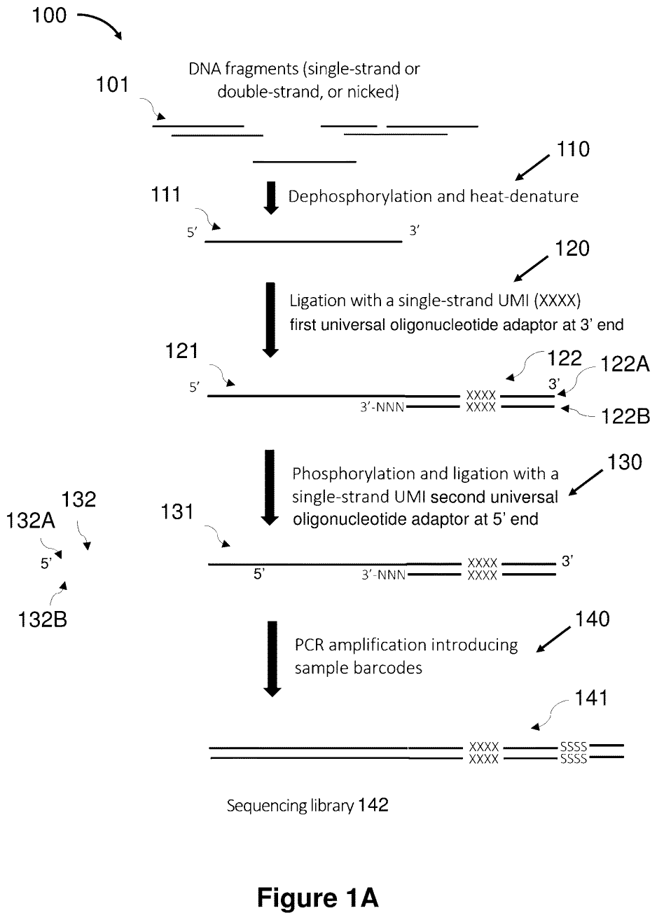

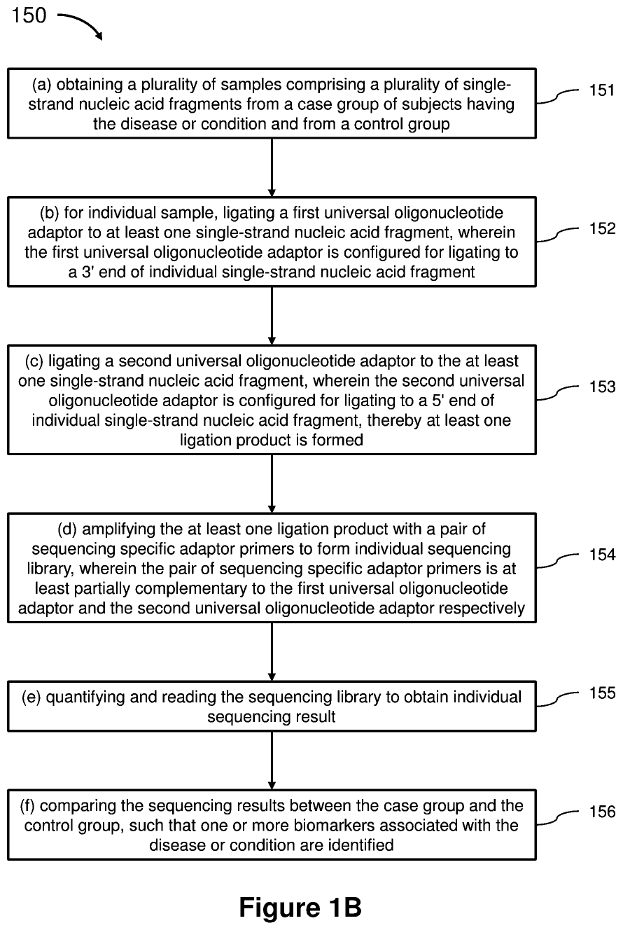

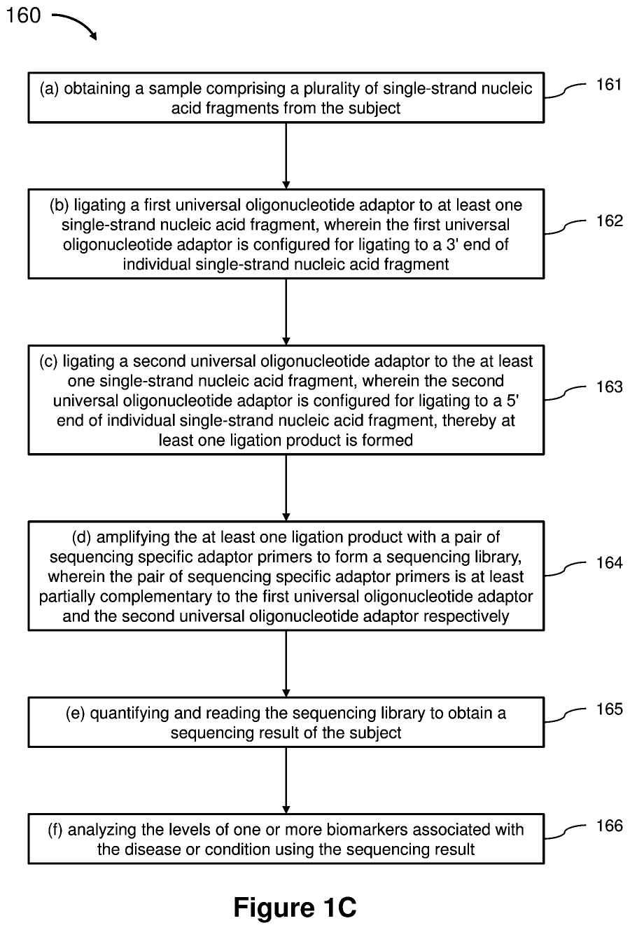

FIG. 1A shows a workflow of an example method 100 for preparing a ligation product and a method of preparing a sequence library from a sample (also referred to as bilateral single-strand sequencing BLESSING in some embodiments). By way of example, the sample is from a mammal, for example, a human. By way of example, the human is a fetus. By way of example, the sample is from a blood sample. By way of example, the sample is cell-free nucleic acids extracted from a blood sample. By way of example, the sample is nucleic acids extracted from circulating tumor cells. By way of example, the sample is nucleic acids extracted from lymphocytes in a blood sample for T-cell and B-cell receptor profiling. In this example, the sample includes a plurality of DNA fragments 101. By way of example, the starting material of the DNA fragments 1001 can be single-strand DNA fragments such as circulating cell-free DNA (ccfDNA), double-strand DNA fragments, and/or nicked DNA fragments. By way of example, the DNA fragments 1001 are prepared from high molecular weight DNA, e.g., genomic DNA. By way of example, the DNA fragments 101 in the sample includes a plurality of single-strand DNA fragments prepared from denaturation of double-strand DNA fragments. By way of example, the DNA fragments 101 in the sample are single-strand cDNA fragments prepared from reverse transcription of RNA fragments.

In this example, in an optional step 110, the 5′ end of individual DNA fragment 1001 is dephosphorylated (for example, by using FastAP (Thermo Scientific)) and optionally heat-denatured to form a 5′ end dephosphorylated single-stranded DNA fragment 111. In step 120, a first universal oligonucleotide adaptor 122 is ligated with the single-stranded DNA fragment 111 at the 3′ end to form a first ligated fragment 121. In an optional step (not shown), the reaction was then cleaned up using paramagnetic beads (such as Agencourt AMPure XP beads) to purify the first ligated fragment 121. In this example, the first universal oligonucleotide adaptor 122 includes a top strand 122A with a 5′ recessive end which is configured for ligating to the 3′ end of the single-stranded DNA fragment 111, and a bottom strand 122B partially complementary to the top strand 122A to form a duplex portion. In some embodiments, the bottom strand 122B includes an unpaired 3′ portion at the 3′ end including multiple number bases of random or degenerate nucleotides, for example, three to twenty. In this example as shown in FIG. 1A, the number of bases of random nucleotides is three (NNN). The two strands in the duplex portion of the first universal oligonucleotide adaptor 122 may be substantially complementary to each other and the duplex portion is of sufficient length to remain in duplex form at the ligation temperature. In some embodiments, the first universal oligonucleotide adaptor 122 further comprise three to twenty random nucleotides (four in this example, shown as XXXX of first universal oligonucleotide adaptor 122 in FIG. 1A) incorporated in the duplex portion as a unique molecular index (UMI) for tracing individual original molecules. In some embodiments, the bottom strand 122B of the first universal oligonucleotide adaptor 122 comprises, consists of or consists essentially of a nucleotide sequence of SEQ ID NO:3, and the top strand 122A of the first universal oligonucleotide adaptor comprises, consists of or consists essentially of a nucleotide sequence of SEQ ID NO:4. In some embodiments, the top strand 122A and the bottom strand 122B is pre-annealed to form the double-stranded, first universal oligonucleotide adaptor 122 before use. In some embodiments, the top strand 122A and the bottom strand 122B are annealed at equal molar using an annealing program on a thermocycler according to manufacturer's protocol to prepare the first universal oligonucleotide adaptor 122 for ligation at 5′ end of single-stranded DNA fragment 111 to form first ligated fragment 121.

In this example, in step 130, the 5′ end of the first ligated fragment 121 is optionally phosphorylated, and a second universal oligonucleotide adaptor 132 is ligated with the first ligated fragment 121 at the 5′ end to form a ligation product 131. After step 130 is performed, the ligation product 131 includes the single-stranded DNA fragment 111, second universal oligonucleotide adaptor 132 ligated to the 5′ end of single-stranded DNA fragment 111, and first ligated fragment 121 ligated to the 3′ end of single-stranded DNA fragment 111. In this example, the second universal oligonucleotide adaptor 132 includes a top strand 132A with a 3′ recessive end which is configured for ligating to the 5′ end of the single-stranded DNA fragment 111, and a bottom strand 132B partially complementary to the top strand 132A to form a duplex portion. In some embodiments, the bottom strand 132B includes an unpaired 5′ portion at the 5′ end including multiple number bases of random or degenerate nucleotides, for example, three to twenty. In this example, the number of bases of random nucleotides is three (NNN). The two strands in the duplex portion of the second universal oligonucleotide adaptor 132 may be substantially complementary to each other and the duplex portion is of sufficient length to remain in duplex form at the ligation temperature. In some embodiments, the second universal oligonucleotide adaptor 132 further comprise three to twenty random nucleotides (four in this example, shown as XXXX of second universal oligonucleotide adaptor 132 in FIG. 1A) incorporated in the duplex portion as a unique molecular index (UMI) for tracing individual original molecules. In some embodiments, the bottom strand of the second universal oligonucleotide adaptor 132 comprises, consists of or consists essentially of a nucleotide sequence of SEQ ID NO:1, and the top strand of the first universal oligonucleotide adaptor comprises, consists of or consists essentially of a nucleotide sequence of SEQ ID NO:2. In some embodiments, the top strand 132A and the bottom strand 132B is pre-annealed to form the double-stranded, second universal oligonucleotide adaptor 132 before use. In some embodiments, the top strand 132A and the bottom strand 132B are annealed at equal molar using an annealing program on a thermocycler according to manufacturer's protocol to prepare the second universal oligonucleotide adaptor 132 for ligation at 5′ end of single-stranded DNA fragment 111 to form ligation product 131.

In some embodiments, after step 130, an optional step (not shown) can be performed to enrich at least one targeted nucleic acid from the ligation product 131 using a target specific primer and a universal oligonucleotide adaptor primer that is at least partially complementary to the first universal oligonucleotide adaptor 122 or second universal oligonucleotide adaptor 132.

In step 140, the ligation product 131 is subsequently amplified by PCR with a pair of sequencing specific adaptor primers (not shown) to form a PCR product 141 that can be used to construct a sequencing library 142. In some embodiments, the pair of sequencing specific adaptor primers (also referred to as adaptor primers) is at least partially complementary to the first universal oligonucleotide adaptor 122 and the second universal oligonucleotide adaptor 132 respectively, so that the same pair of sequencing specific adaptor primers can be used to amplify different single-stranded DNA fragments from the sample. By ways of example, the pair of sequencing specific adaptor primers are Illumina adaptor primers. By way of example, the pair of sequencing specific adaptor primers may include one or more sample barcodes (shown as SSSS in FIG. 1A) in one or both of the adaptor primers for tracing individual samples. The one or more sample barcodes are introduced into the PCR product 141 during PCR amplification in step 140. By way of example, the PCR product 141 can be further purified by paramagnetic beads, such as Agencourt AMPure XP beads. By way of example, the sequencing library 142 may be used for subsequent sequencing step with a sequencing primer pair, which is at least partially complementary to opposite strands of the PCR product 142, respectively. By way of example, the sequencing library 142 can be quantified by real-time PCR (such as with KAPA Library Quantification Kits for Illumina System) and sequenced on a sequencing platform (such as the NovaSeq 6000 System from Illumina).

### Example 2: Example Workflow of a Method of Identifying One or More Biomarkers Associated with a Disease or Condition

FIG. 1B is a flowchart of an example method 150 of identifying one or more biomarkers associated with a disease or condition.

Block 151 states obtaining a plurality of samples comprising a plurality of single-strand nucleic acid fragments from a case group of subjects having the disease or condition and from a control group.

Block 152 states for individual sample, ligating a first universal oligonucleotide adaptor to at least one single-strand nucleic acid fragment, wherein the first universal oligonucleotide adaptor is configured for ligating to a 3′ end of individual single-strand nucleic acid fragment.

Block 153 states ligating a second universal oligonucleotide adaptor to the at least one single-strand nucleic acid fragment, wherein the second universal oligonucleotide adaptor is configured for ligating to a 5′ end of individual single-strand nucleic acid fragment, thereby at least one ligation product is formed.

Block 154 states amplifying the at least one ligation product with a pair of sequencing specific adaptor primers to form individual sequencing library, wherein the pair of sequencing specific adaptor primers is at least partially complementary to the first universal oligonucleotide adaptor and the second universal oligonucleotide adaptor respectively.

Block 155 states quantifying and reading the sequencing library to obtain individual sequencing result.

Block 156 states comparing the sequencing results between the case group and the control group, such that one or more biomarkers associated with the disease or condition are identified. By way of example, the one or more biomarkers identified can be used for predicting or detecting the disease or condition in a given subject.

### Example 3: Example Workflow of a Method of Predicting or Detecting a Disease or Condition in a Subject

FIG. 1C is a flowchart of an example method 160 of predicting or detecting a disease or condition in a subject. By way of example, the method can be used for predicting prognosis in a subject with a disease or condition such as cancer. By way of example, the method can be used for early detection or diagnosis of a disease or condition such as cancer in a subject. By way of example, the cancer is hepatocellular carcinoma (HCC).

Block 161 states obtaining a sample comprising a plurality of single-strand nucleic acid fragments from the subject.

Block 162 states ligating a first universal oligonucleotide adaptor to at least one single-strand nucleic acid fragment, wherein the first universal oligonucleotide adaptor is configured for ligating to a 3′ end of individual single-strand nucleic acid fragment.

Block 163 states ligating a second universal oligonucleotide adaptor to the at least one single-strand nucleic acid fragment, wherein the second universal oligonucleotide adaptor is configured for ligating to a 5′ end of individual single-strand nucleic acid fragment, thereby at least one ligation product is formed.

Block 164 states amplifying the at least one ligation product with a pair of sequencing specific adaptor primers to form a sequencing library, wherein the pair of sequencing specific adaptor primers is at least partially complementary to the first universal oligonucleotide adaptor and the second universal oligonucleotide adaptor respectively.

Block 165 states quantifying and reading the sequencing library to obtain a sequencing result of the subject.

Block 166 states analyzing the levels of one or more biomarkers associated with the disease or condition using the sequencing result. By way of example, the one or more biomarkers associated with the disease or condition are identified by the method 150 as disclosed in Example 2 above.

### Methods and Materials

A prospective cohort with hepatitis B virus (HBV)-seropositive participants were enrolled in 2012 and followed-up biannually with blood sample collections till 31 Dec. 2019. A case-control study with hospital hepatocellular carcinoma (HCC) cases were conducted to identify potential biomarkers for HCC detection (Discovery). A technology termed bilateral single-strand sequencing (BLESSING) was developed for circulating cell-free DNA (ccfDNA) analysis. A telomere and end sequence phenomenon etymology (Telephone) model was built for detecting HCC at the Discovery phase and Telephone was validated in the HBV-seropositive cohort-nested case-control study (Validation).

### Example 4: Study participants

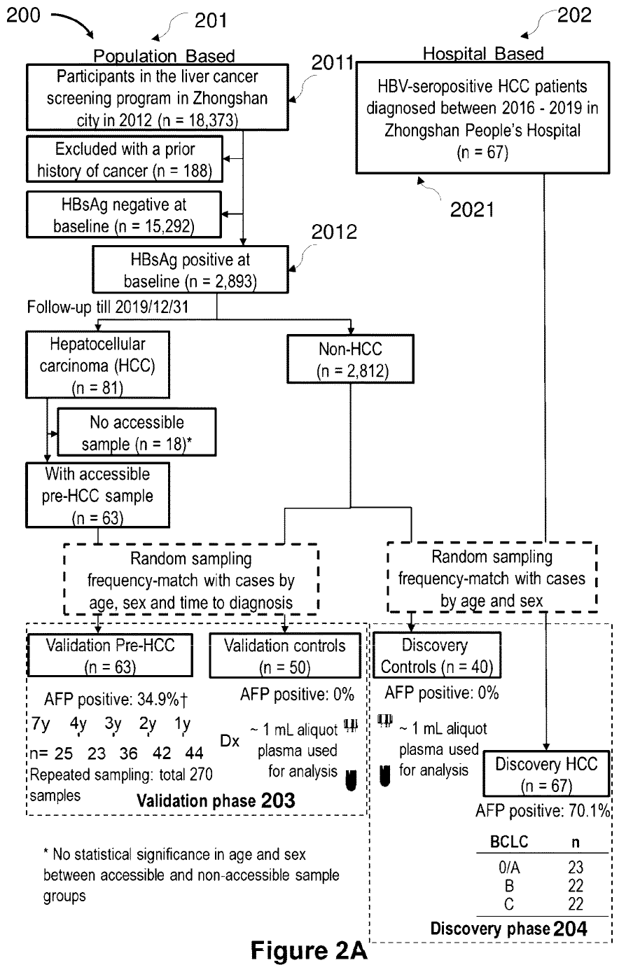

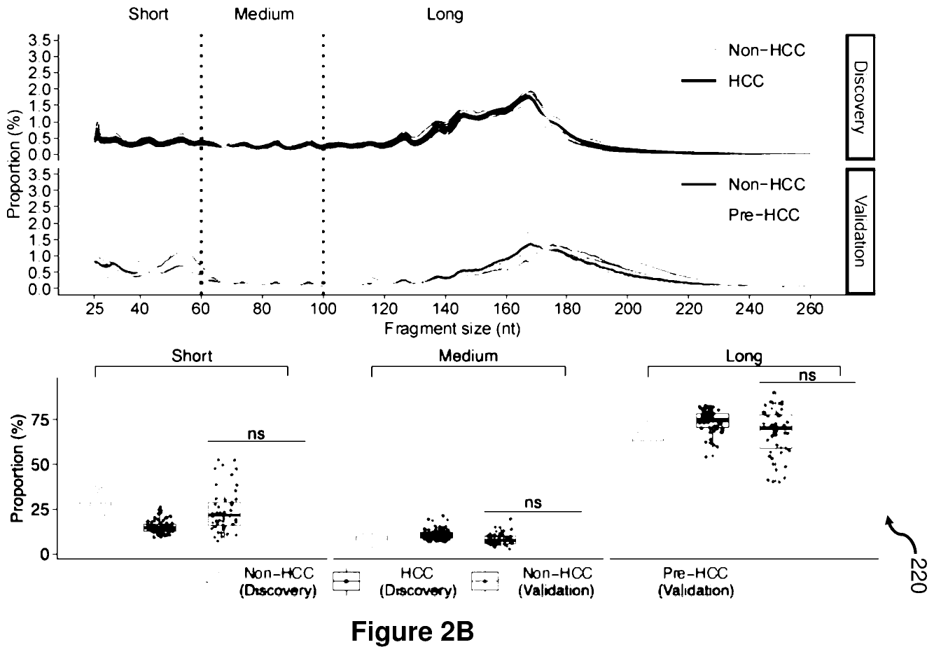

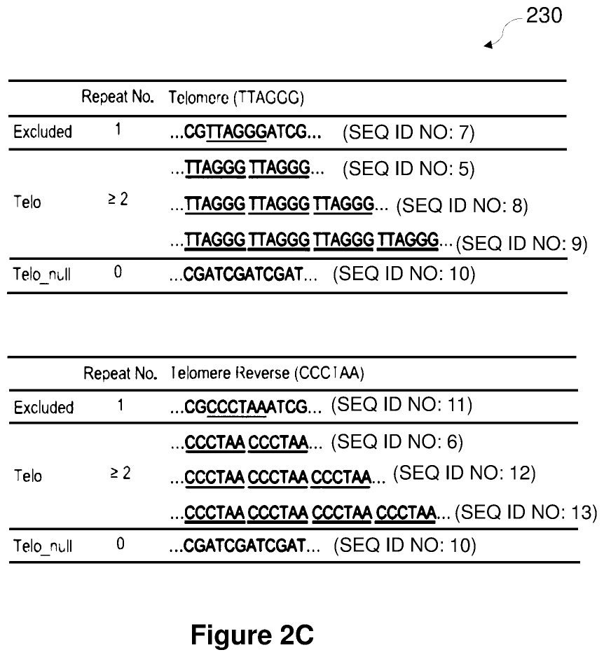

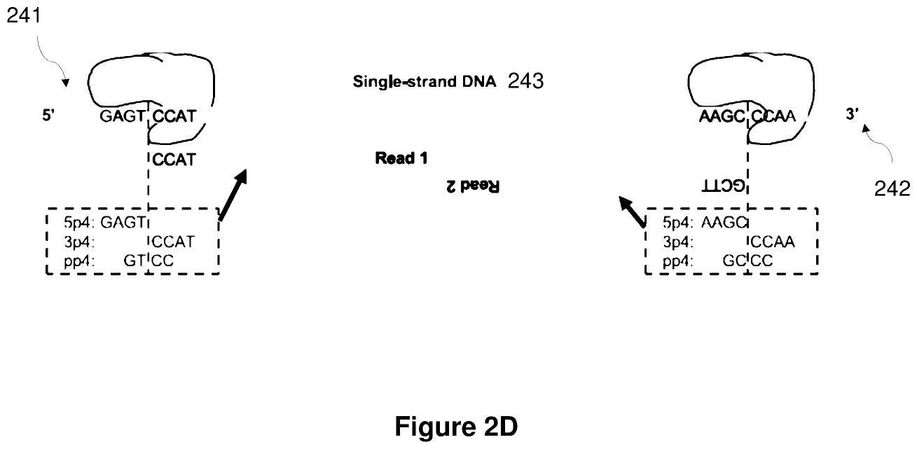

Now referring to FIG. 2A, which illustrates an example workflow 200 of a study consisted of a population-based cohort 201 for validation (validation phase 203) and a hospital-based study 202 (discovery phase 204) for initial biomarker identification according to an example embodiment. A liver cancer screening trail in Zhongshan City started participant enrollment in 2012 (NCT02501980, ClinicalTrials.gov) (Block 2011). At baseline, all participants were tested for HBsAg. HBV-seropositive individuals (Block 2012) were subjected to biannual follow-up and serial blood samples were collected. These HBV-seropositive subjects were followed-up till Dec. 31, 2019, and their disease status were retrieved from local hospitals and Cancer Registry. Based on this HBV-seropositive cohort, a nested case-control study were performed where incident HCC cases were matched with non-HCC controls by sex, age (±1 year), and date of blood sample collection time (±3 months).

To first identify potential biomarkers for early detection of HBV-related HCC, patients who were HbsAg-seropositive and newly diagnosed in Zhongshan People's Hospital, Zhongshan City, China between 2016 and 2019 (Block 2021) were invited to participate in the study (Discovery phase 204). Cases were oversampled with early stages (Barcelona Clinic Liver Cancer [BCLC] stage 0 or A) and plasma samples were collected from 67 HBV-related HCC cases (34% of which were in BCLC stage 0 or A) in the study. In addition, 40 sex and age matched community controls who were positive for HbsAg test were randomly selected. All samples were obtained under Institutional Review Board approved protocols and with informed consent from all participants for research use.

### Example 5: Blood Sample Preparation and DNA Extraction

Blood samples collected from the screening cohort at each screening visit were performed as follows: venous peripheral blood was collected in one K2-EDTA tube and one serum gel tube. Within 24 hours after storage at 4° C., blood collection tubes were centrifuged at 1600×g at room temperature for 10 min. After centrifugation, plasma, buffy coat and serum samples were stored at −20° C. for future analyses. Plasma samples obtained at the time of diagnosis for hospital HCC cases were performed as follows: venous peripheral blood was collected in one K2-EDTA tube and two serum gel tubes. Within two hours from blood collection, tubes were centrifuged at 1600×g at room temperature for 10 min. Supernatant plasma and buffy coat were separated and the plasma was centrifuged second time at 16000×g at 4° C. for 10 min to remove remaining cellular debris. After centrifugation, plasma samples were stored at −80° C. before analyses. For all samples, about ˜1 mL plasma was used for cfDNA extraction, excepted in 10 samples only 0.5 mL was available. Plasma cfDNA was isolated using the QIAamp® MinElute® ccfDNA Mini Kit (Cat. No. 55284, QIAGEN, Germantown, MD) following the manufacturer's protocol. DNA concentration was measured by Qubit 3 Fluorometer (ThermoFisher).

### Example 6: Bilateral Single-Strand Sequencing (BLESSING)

Now referring back to FIG. 1A, which shows an example workflow of a method for preparing a ligation product and a sequence library which is termed as bilateral single-strand sequencing (BLESSING). In this embodiment, at step 110, extracted DNA was first de-phosphorylated using FastAP (Thermo Scientific) and incubated at 37° C. for 15 min, 75° C. for 10 min and 95° C. for 3 min and immediately cooled down on ice-water. Next, in step 120, the product (single-stranded DNA fragment 111) was ligated with a unique molecule index (UMI)-containing first universal oligonucleotide adaptor 122 that can ligate the 3′ end of single-stranded DNA fragment 111 to form first ligated fragment 121. The reaction was then cleaned up using 1.5× Agencourt AMPure XP beads. In step 130, the purified product (first ligated fragment 121) was then phosphorylated by T4 Polynucleotide Kinase with ATP and incubated at 37° C. for 30 min, 65° C. for 20 min, 95° C. for 3 min and immediately cooled on ice-water, followed by ligation with another UMI-containing second universal oligonucleotide adaptor 132 that can ligate to the 5′ end of first ligated fragment 121 to form ligation product 131. Finally, in step 140, the ligation product 131 was amplified by 10 cycles of PCR using sequencing platform (Illumina) adaptor primers with sample barcodes to form PCR product 141 and purified by 1.0×Agencourt AMPure XP beads. The resulting library (sequencing library 142) was quantified by real-time PCR with the KAPA Library Quantification Kits for Illumina System and sequenced on the NovaSeq 6000 System.

### Example 7: First and Second Universal Oligonucleotide Adaptors

Table 1 summarizes the first universal oligonucleotide adaptor sequences (bottom strand ss7B, and top strand ss7T) and the second universal oligonucleotide adaptor sequences (bottom strand ss5B, and top strand ss5T) used in preparation of the single stranded sequencing libraries by BLESSING according to an example embodiment (such as Example 5). The ss7B and ss7T oligos were annealed at equal molar using a regular annealing program on thermocycler to prepare the first universal oligonucleotide adaptor for ligation at 3′ end of single-stranded template. The ss5B and ss5T were pre-annealed to form the second universal oligonucleotide adaptor before use. The ss5B and ss5T oligos were annealed at equal molar using a regular annealing program on thermocycler to prepare the second universal oligonucleotide adaptor for ligation at 5′ end of single-stranded template.

TABLE 1
Synthetic oligos used in the preparation of single stranded
sequencing libraries by Bilateral single-strand sequencing
(BLESSING) according to an example embodiment and their
purification methods. N = A, C, G, or T. W = A or T, * =
phosphorothioate bond. /5Phos/ = 5′ phosphorylation.

| Oligo | | |
|---|---|---|
| Name | Sequence (5′-3) | Purification |
| ss7B | A*A*A*TTGACTGGAGTTCAGACGTGTGCTCTTCCGAT | HPLC |
| | CTNNWNNWNNAGACTCTGNNNNNN (SEQ ID NO: 3) | |
| ss7T | /5Phos/CAGAGTCTNNWNNWNNAGATCGGAAGAGCAC | HPLC |
| | ACGTCTGAACTCCAGT*C*A*A (SEQ ID NO: 4) | |
| ss5B | NNNNNNGTGTCACTNNWNNNWNNNAGATCGGAAGA | HPLC |
| | GCGTCGTGTAGTT (SEQ ID NO: 1) | |
| ss5T | A*A*C*TACACGACGCTCTTCCGATCTNNNWNNNWNN | HPLC |
| | AGTGACAC (SEQ ID NO: 2) | |

### Example 8: Bioinformatic and Biostatical Analyses

Raw FASTQ data was de-multiplexed using bcl2fastq2, trimmed adaptors using BBDuk, and further extracted 5′ and 3′ UMIs using inhouse scripts. Reads with incorrect UMI lengths were excluded from downstream analyses. The cleaned FASTQ sequences were aligned to human reference genome (hg38) using BWA MEM.

### Telomere and End Sequence Phenomenon Etymology (Telephone)

Referring now to FIG. 2C. Telomere sequences as shown in table 230 were identified from the cleaned FASTQ data. Human telomere contains the characteristic sequence 5′-TTAGGG-3′. Sequence containing only single 5′-TTAGGG-3′ was excluded from analysis to reduce misclassification due to random occurrence the short segment in non-telomere DNA fragments. Sequences with at least two consecutive telomere repeats 5′-TTAGGGTTAGGG-3′ (SEQ ID NO: 5) were therefore defined as telomere-containing sequences, referred to as “Telo”, and sequences do not contain 5′-TTAGGG-3′ as non-telomere (“Telo_null”). Since BLESSING is aware of strand direction, similarly, sequences with at least two consecutive telomere reverse complementary sequence 5′-CCCTAACCCTAA-3′ (SEQ ID NO: 6) were defined as telomere reverse sequence-containing sequences, referred to as “TeloRv”, and sequences do not contain 5′-CCCTAACCCTAA-3′ (SEQ ID NO: 6) as non-telomere reverse sequences (“TeloRv_null”).

Now referring to FIG. 2D. For DNA fragment ends, 4 bases were first extracted at the 5′ end 241 and 3′ end 242 of single-strand DNA fragments 243, designated “5p4” and “3p4”, respectively. DNA ends may be a result of restriction enzyme digestion, and the recognition sequence may flank the cutting site (e.g., NN|NN, where “|” represents the cutting site). Because DNA sequencing library is prepared by ligating adaptors to cut DNA fragment ends, one sequence read contains only one end of the cutting site (“NN|” or “|NN”), the full 4-base recognition sequence was inferred by adding the un-sequenced end after aligning the sequence to human reference genome, and designated as “pp4”. Thus, three types of 4-nt end sequences (5p4, 3p4, pp4) were included in the analyses. Furthermore, as BLESSING is aware of fragment direction, the end sequences were further separated by end source (5′ or 3′ of a DNA fragment). DNA fragment length was inferred from chromosome coordinates of paired-end alignments. Given that BLESSING can sequence very short DNA, fragments were categorized into short (25 to 60 nt), medium (61 to 100 nt) and long (≥101 nt) groups.

At the Discovery phase, potential biomarkers for detecting HCC were first identified. Proportions of telomeres and end sequences were compared between cases and controls using Wilcoxon rank-sum test. Candidate markers with fold-difference (case vs control) ≥2 or ≤0.5 were then selected. Unsupervised hierarchical clustering analysis was performed using the top selected features with Manhattan distance and centroid linkage. Among these potential markers, markers demonstrated the greatest ability to accurately discriminate between cases and controls were evaluated using logistic regression model with a Least Absolute Shrinkage and Selection Operator (LASSO) penalty. The optimal value of lambda (λ) penalty with 5-fold cross-validation was determined by resampling using the caret R package. A candidate marker was selected if its coefficients was non-zero. Based on the selected markers at Discovery phase, a logistic regression model was formulated using LASSO coefficients, named Telomere and end sequence phenomenon etymology (Telephone), for detecting early HCC.

### Independent Validation of Telephone in a Prospective Cohort

Sensitivity, specificity, and area under curve (AUC) were used to evaluate diagnostic performance. Positive predictive value (PPV) and negative predictive value (NPV) were estimated in a population setting where male chronic HBV carriers has an incidence rate of 525 per 100,000 person-years for HCC.

Association of Clinical Covariates and Survival with Telephone

The distribution of Telephone by sex, age at diagnosis, clinical BCLC stage, and AFP level at diagnosis were compared using a Wilcoxon signed-rank test. Overall survival time was calculated from the date of diagnosis until the date of death or last follow-up if a participant was still alive. To assess whether Telephone was associated with overall survival, Telephone was categorized into high and low groups among the 67 hospital HCC cases. Survival curves were estimated using the Kaplan-Meier method and compared by the log-rank test, with further stratification by the BLCL stage. Telephone was evaluated whether it was independently associated with overall survival in a multivariable Cox proportional hazards model that include age at diagnosis, sex, clinical stage, and AFP level.

### Motif Diversity Score (MDS)

To analyze the distribution of end sequences, a similar method as described by Jiang et. al. (Jiang P, Sun K, Peng W, et al. Plasma DNA end-motif profiling as a fragmentomic marker in cancer, pregnancy, and transplantation. Cancer Discov. 2020; 10(5):664-673. Doi:10.1158/2159-8290.CD-19-0622) was adopted and calculated a normalized Shannon entropy score using 5′ end sequences derived from DNA fragments with length >60 nt.

The normalized Shannon entropy was adopted as a mathematical approach for calculating the MDS. MDS was defined using the following equation:

$$MDS {\sum }_{i = 1}^{256} - {P}_{i}^{*} log ( {P}_{i} ) / log ( 256 )$$

where Pi is the frequency of a particular end sequence. A higher MDS value indicates a higher diversity (i.e., a higher degree of randomness). The theoretical scale is ranged from 0 to 1.

All P values were two-sided. Statistical analyses were conducted using R version 4.0.3. A P value of less than 0.05 after Bonferroni correction for multiple testing was considered statistically significant.

### Results

### Example 9: Study Participants

Now referring to FIG. 2A. In 2012, 18,373 participants were recruited in a population-based liver cancer screening trail in Zhongshan, China (see Block 2011). After excluding 188 subjects with prior history of cancers, 2,893 (15.9%) were seropositive for HBsAg (see Block 2012). Referring to Table 2, the HBsAg-seropositive cohort consisted of more males (68.7%) than females, with a mean age of 48.5. The HBsAg-positive subjects were followed-up every six months, with 81 subjects received HCC diagnoses during follow-up by Dec. 31, 2019, and 2,812 subjects did not. Among the 81 HCC subjects, a total of 270 pre-HCC blood samples were available from 63 subjects (mean age at diagnosis 55.7; males 58 [92.1%]; FIG. 2A), with the numbers of samples collected at the intervals of 4 or more years, 3-4 years, 2-3 years, 1-2 years, and within 1 year before diagnosis 25, 23, 36, 42, and 44, respectively. Referring to Table 3, The remaining 18 HCC subjects had no accessible samples but had no differences with the 63 cases on age and sex distributions. A nested case-control sampling within the HBsAg cohort was performed. A total of 50 samples from 50 non-HCC HBsAg-positive subjects were randomly selected from 28,385 samples of the 2,812 subjects to frequency-match with the 63 HCC cases by age, sex, and sample collection time to diagnosis or end of follow-up. Referring to Table 4, the HCC and non-HCC subjects had comparable age and sex distributions. The AFP positive rate was 34.9% in the HCC group and 0% in the non-HCC group (FIG. 2A). This population-base prospective sample collection cohort served as the basis for later validation (Validation phase).

Hospital HCC cases (Block 202) were used for initial biomarker identification (Discovery phase 204, FIG. 2A). Blood samples at diagnosis from 67 HBsAg-positive HCC patients were recruited (mean age 55.2; males 59 [88.1%]). The number of cases for BCLC Stages 0/A, B and C were 23, 22 and 22, respectively, comparable with the stage distribution in the Validation phase (P=0.081). For HBV-carrier controls, 40 non-HCC subjects were randomly selected from the population HBsAg-seropositive cohort with sample collection at least 1 year, except one case being 6 months, prior to the end of follow-up. The AFP positive rate was 70.1% in the HCC group and 0% in the control group (FIG. 2A).

TABLE 2
Baseline characteristics of liver cancer
screening cohort in Zhongshan

Population screening cohort

| | HBsAg− | HBsAg+ | |
|---|---|---|---|
| | (n = 15,292) | (n = 2,893) | P |
| Age, mean (SD) | 50.9 (7.88) | 48.5 (7.82) | <0.001 |
| Age, n (%) | | | |
| 35-39 | 439 (15.2%) | 1,455 (9.5%) | <0.001 |
| 40-49 | 1,190 (41.1%) | 5,460 (35.7%) | |
| 50-59 | 983 (34.0%) | 5,806 (38.0%) | |
| 60-64 | 281 (9.7%) | 2,571 (16.8%) | |
| Sex, n (%) | | | |
| Female | 9,024 (59.0%) | 905 (31.3%) | <0.001 |
| Male | 6,268 (41.0%) | 1,988 (68.7%) | |

TABLE 3
Age and sex of HCC subjects with or
without accessible pre-HCC samples.

pre-HCC samples

| | Accessible | No accessible | |
|---|---|---|---|
| | (n = 63) | (n = 18) | P |
| Age, mean (SD) | 52.0 (6.38) | 55.1 (4.72) | 0.056 |
| Sex, n (%) | | | |
| Female | 5 (7.94%) | 0 (0%) | 0.582 |
| Male | 58 (92.06%) | 18 (100.0%) | |

TABLE 4
Baseline characteristics of discovery and validation phase.

| | Discovery phase | | | | | Validation phase | | | | |
|---|---|---|---|---|---|---|---|---|---|---|
| | HCC | | | | | Pre-HCC | | | | |
| | patients | | Non-HCC | | | patients | | Non-HCC | | |
| | (n = 67) | | (n = 40) | | P | (n = 63) | | (n = 50) | | P |
| Age, mean (SD) | 55.2 | (9.43) | 55.1 | (6.53) | 0.926 | 55.7 | (6.52) | 55.7 | (7.21) | 0.956 |
| Sex, n (%) | | | | | | | | | | |
| Female | 8 | (11.9%) | 4 | (10.0%) | 1 | 5 | (7.9%) | 4 | (8.0%) | 1 |
| Male | 59 | (88.1%) | 36 | (90.0%) | | 58 | (92.1%) | 46 | (92.0%) | |
| AFP, n (%) | | | | | | | | | | |
| Negative | 20 | (29.9%) | 40 | (100%) | <0.001 | 41 | (65.1%) | 50 | (100%) | <0.001 |
| Positive | 47 | (70.1%) | 0 | (0%) | | 22 | (34.9%) | 0 | (0%) | |

| | | | | | | | |
|---|---|---|---|---|---|---|---|
| BCLC | | | | | | | |
| stage, n (%) | | | | | | | |
| 0/A | 23 | (34.3%) | — | 21 | (33.3%) | — | 0.081* |
| B | 22 | (32.8%) | — | 19 | (30.2%) | — | |
| C | 22 | (32.8%) | — | 16 | (25.4%) | — | |
| D | 0 | (0%) | — | 5 | (7.9%) | — | |
| Unknown | 0 | (0%) | — | 2 | (3.2%) | — | |

*Fisher's exact test P value for BCLC stage among HCC patients between discovery and validation phase.

### Example 10: Circulating Cell-Free (ccfDNA) and Telomere Profiles

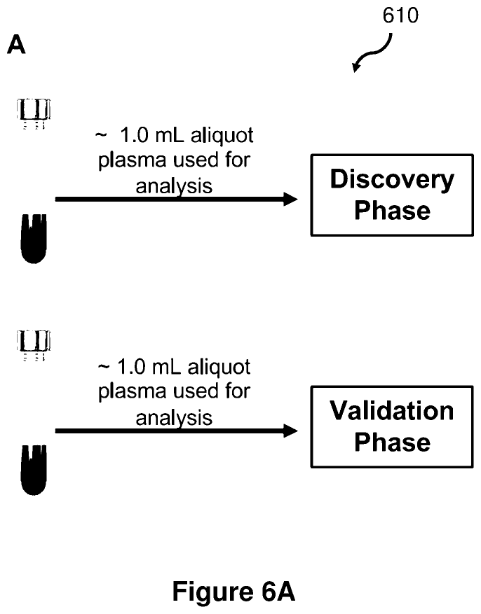

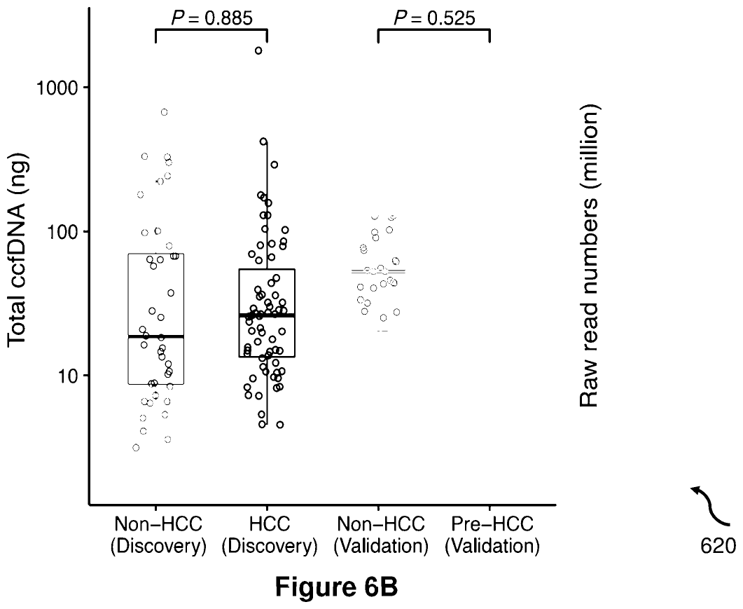

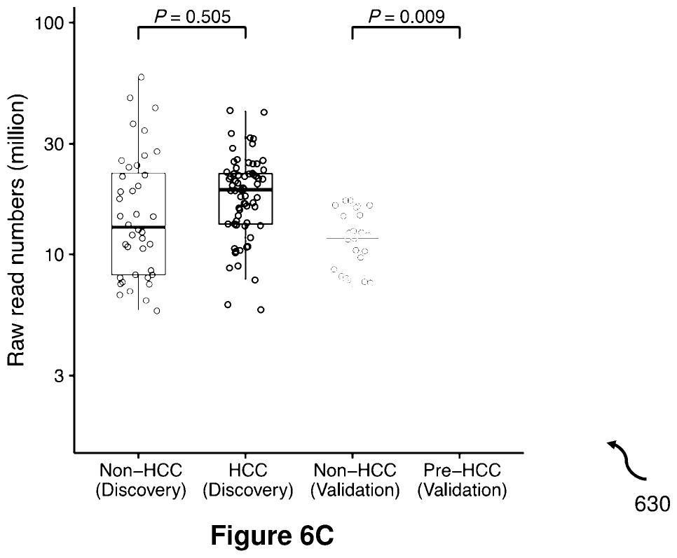

To maximally recover ccfDNA including those of ultra-short sizes and to preserve nature DNA fragment ends in biological samples, a simple and direct whole genome sequencing library construction method were developed, termed 2bilateral single-strand sequencing (BLESSING). About 1 mL of plasma from all study subjects was used. Referring to FIG. 6B, the total ccfDNA amount of non-HCC and HCC/Pre-HCC in discovery and validation phases is shown in graph 620. The yield of ccfDNA was comparable between HCC cases and controls in both Discovery (median 79.8 ng vs 74.8 ng) and Validation phases (median 114 ng vs 98.9 ng, both P values >0.05). Referring to FIG. 6C, the raw read numbers of sequencing data of non-HCC and HCC/Pre-HCC in discovery and validation phases are shown in graph 630. Number of sequencing reads were comparable between HCC patients and controls in the Discovery phase (median 19.0 million vs 17.7 million, P=0.505) but was higher in pre-HCC patients than in controls in the Validation phase (median 20.9 million vs 15.6 million, P=0.009).

Referring now to FIG. 2B, the size distributions of ccfDNA fragments in discovery and validation phases are shown in graph 220. The size distribution of ccfDNA fragments showed two dominant peaks at 167 nt and 53 nt and minor peaks regularly spaced every 10 nt in most subjects. The proportion of short fragments (25 to 60 nt) was higher in controls than in HCC cases in the Discovery phase (27.6% vs 15.1%, P<0.001). Among the long fragment group (≥101 nt), HCC cases had shorter fragments than controls in the Discovery phase (mean±SD: 154.6±24.0 vs 175.6±26.9, P<0.001), whereas only relatively small difference was observed comparing pre-HCC and non-HCC in the Validation phase (170.1±26.8 vs 174.3±27.3, P<0.001). Telomere sequences (0230) were extracted in forward (Telo: TTAGGG) and reverse (TeloRv: CCCTAA) directions, and 4-base DNA fragment end sequences at 3′ end (3p4), at 5′ end (5p4), and 2 genome-inferred bases plus 2 sequenced fragment-end bases in the 5′ to 3′ direction (pp4) using custom bioinformatic algorithms (refer to FIG. 2C and Methods as described in Example 7).

### Example 11: Marker Selection and Modeling for Early Detection of HCC

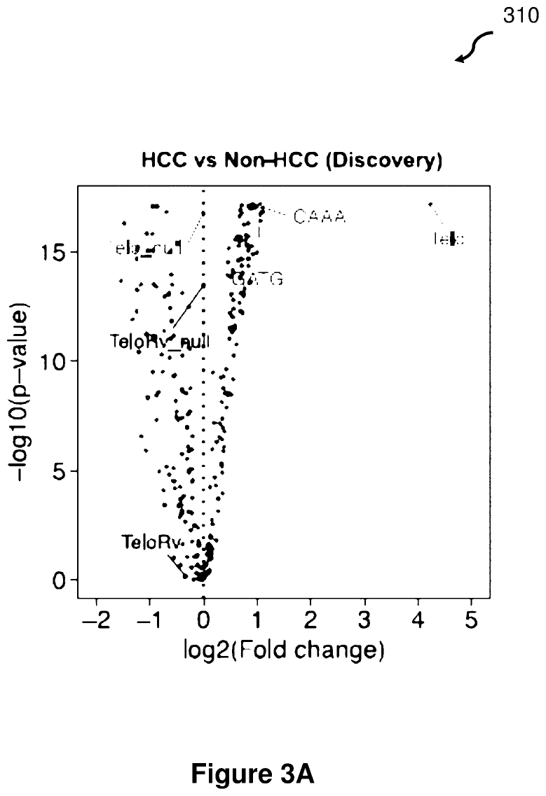

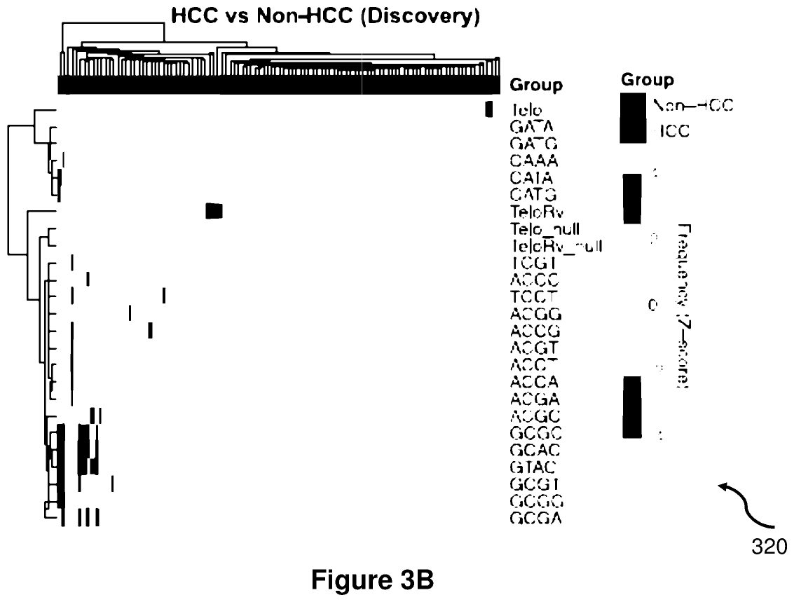

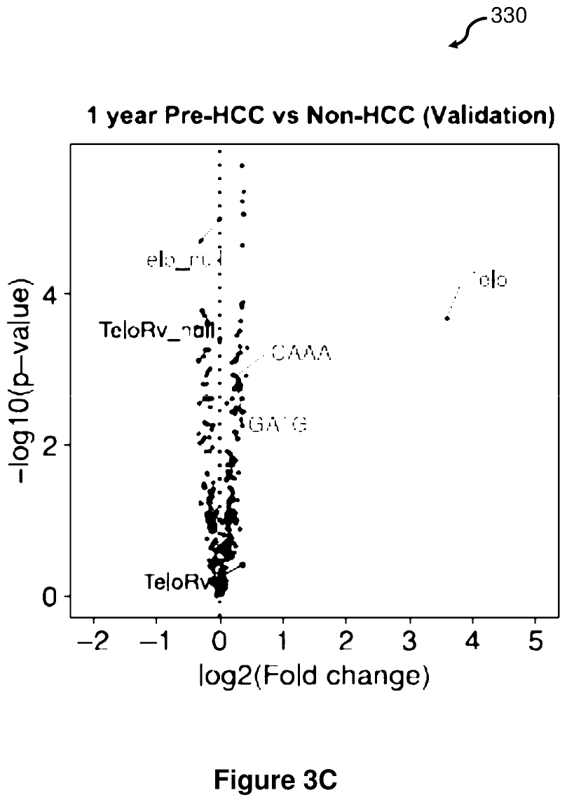

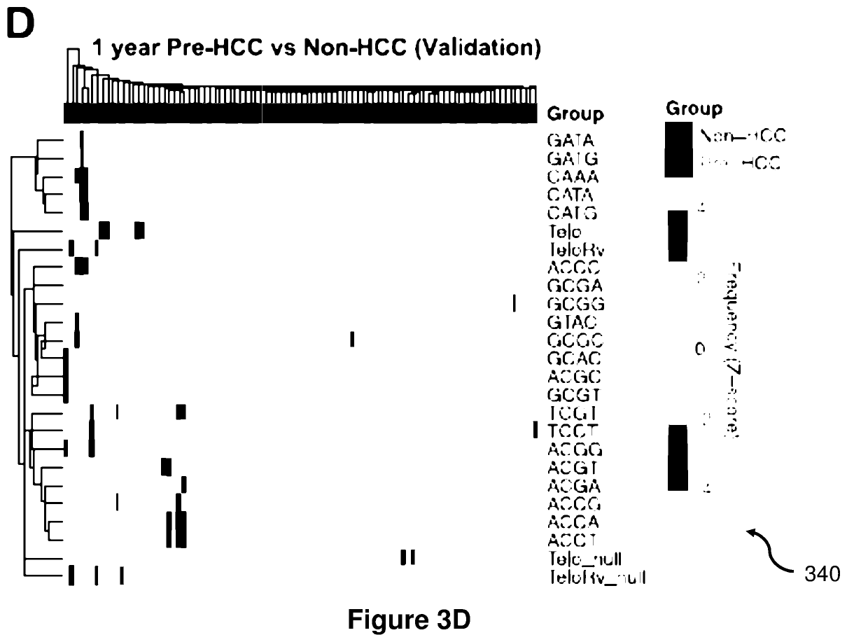

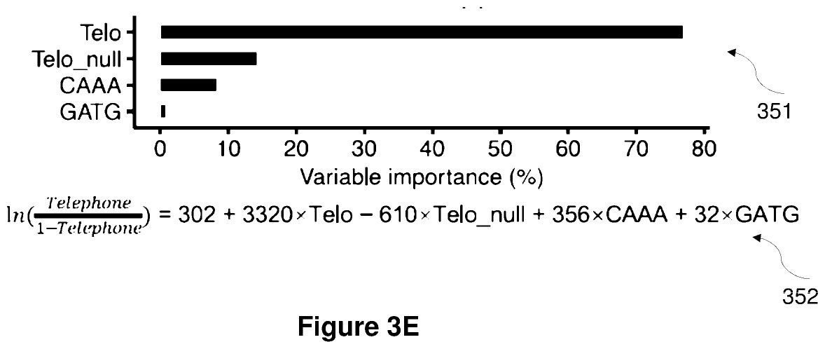

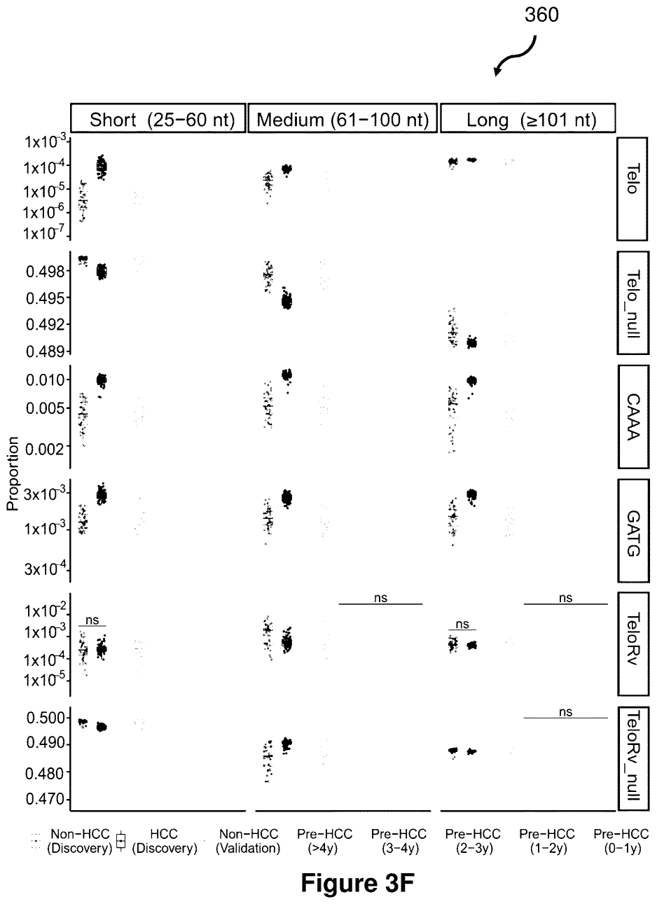

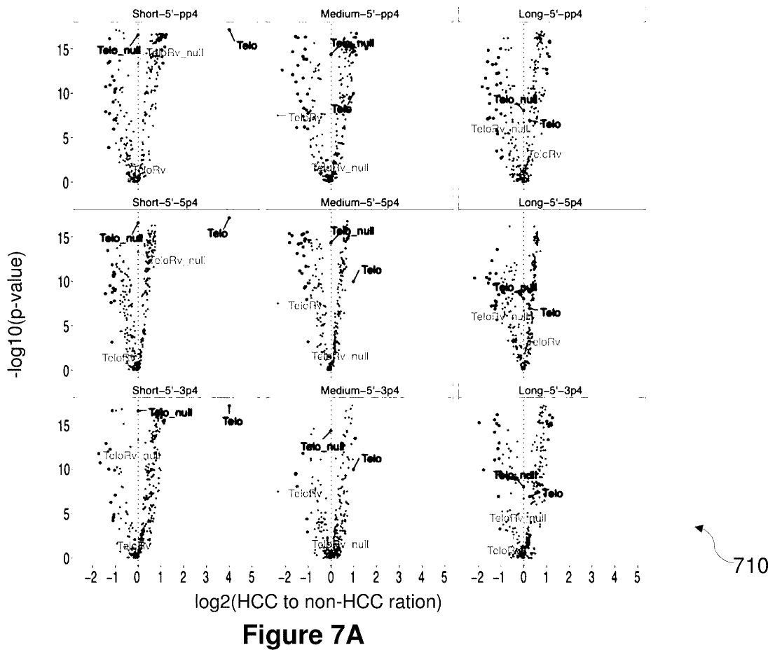

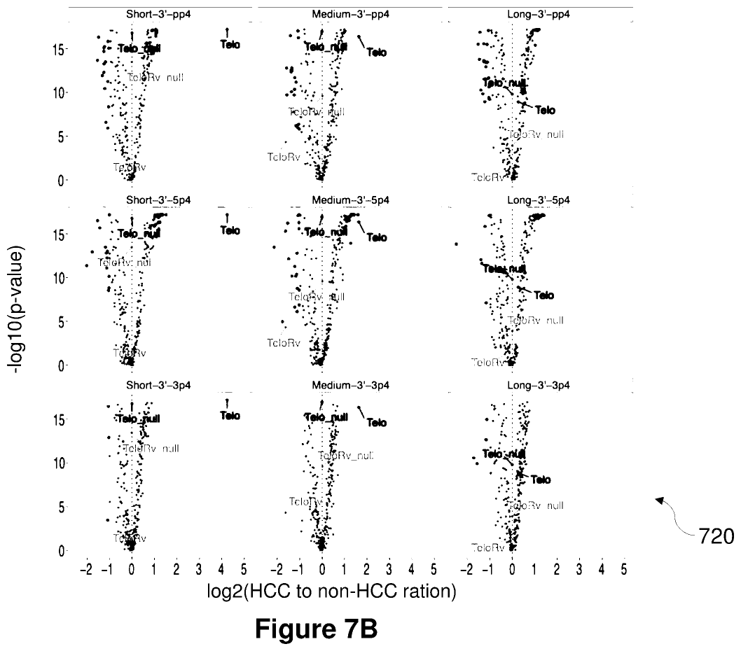

The proportions of telomere (Telo) and non-telomere (Telo_null) fragments 310, their reverse complement fragments (TeloRv and TeloRv_null), and fragment end sequences (256 possible 4-nt sequences) were compared between HCC and control groups in the Discovery phase. The comparisons were stratified by fragment end source (5′/3′), fragment size (short/medium/long) and type of end sequence (5p4/3p4/pp4), yielding 18 stratifications in total. Referring to Table 5, in the Discovery phase, based on markers derived from short fragments, 3′ fragment end source and the ‘pp4’ type end sequences (“short-3′-pp4” stratum), 187 out of total 260 markers showed different proportions between HCC and controls after Bonferroni-correction for multiple testing. Referring now to FIG. 3A, a graph 310 shows case-control comparison of telomere (Telo) and non-telomere (Telo_null) fragments (0310), their reverse complement fragments (TeloRv and TeloRv_null), and fragment end sequences between HCC and non-HCC control groups in terms of p-value versus fold change in the Discovery phase. Compared with controls, markers that were significantly higher in HCC included telomere (fold-difference 18.87, P=6.4×10⁻¹⁸), CAAA (2.16, P=9.5×10⁻¹⁸), and GATG (2.09, P=6.4×10⁻¹⁸); and significantly lower markers included non-telomere containing fragments (0.997, P=1.66×10⁻¹⁷), TCCA (0.52, P=8.49×10⁻¹⁸), and GCCA (0.62, P=1.48×10⁻¹⁷) (FIG. 3A and Table 5). Telomere-related markers and end sequences that showed a fold difference of ≥2 or ≤0.5 (N=25) were selected for hierarchical clustering analysis. As shown in graph 320 in FIG. 3B, the result showed excellent separation of HCC cases and controls. Referring now to FIGS. 7A and 7B, graphs 710 and 720 show case-control comparisons of 260 telomere and 4-nt end sequences in discovery phase among 18 strata 0710, 0720, namely by fragment size (short/medium/long), end source (5′/3′), and type of end sequence (5p4/3p4/pp4). Case-control comparisons of the markers derived from these strata (fragment size: medium or long; type of end sequence: 5p4 or 3p4; fragment end source: 5′) showed similar results but were less significant than the markers derived from the short-3′-pp4 stratum (FIGS. 7A and 7B). Hence, this stratum was focused on in the following analyses. For Validation phase samples pre-HCC samples collected 1 year 6 months before diagnosis were first focused on, resulting in 43 pre-HCC samples collected 6.4-17.9 months before diagnosis. Referring now to FIG. 3C, a graph 330 shows case-control comparison of the proportions of telomere (Telo) and non-telomere (Telo_null) fragments, their reverse complement fragments (TeloRv and TeloRv_null), and fragment end sequences between 1 year Pre-HCC and non-HCC control groups in terms of p-value versus fold change in the Validation phase. Of the 260 markers evaluated, only 12 showed differences when comparing the 1-y pre-HCC samples (N=43) and matched controls (N=50). Strikingly, telomere remained significantly different between the groups (fold difference 12.08, P=2.05×10⁻⁴). Referring now to graph 340 in FIG. 3D, the hierarchical clustering analysis based on the same 25 markers as those selected in the Discovery phase did not show clear separation of 1-y pre-HCC and controls.

Next, based on the 25 markers identified from the Discovery phase and LASSO modeling, a biomarker model was built for early detection of HCC, resulted in a model the inventors named Telephone (Telomere and End sequence Phenomenon Etymology). Referring now to FIG. 3E, which shows a graph 351 comparing the example variable importance of Telephone markers and an example equation 352 to calculate a Telephone score to express the contributions of 4 markers. Telephone included 4 markers 0351, two telomere related (Telo and Telo_null) and two end sequences (pp4 at 3′ end: CAAA and GATG), with their contributions to Telephone being 76.9%, 14.1%, 8.3% and 0.7%, respectively, and expressed as

$$ln ( \frac{Telephone}{1 - Telephone} ) = 302 + 3320 \times Telo - 610 \times Telo\_null + 356 \times CAAA + 32 \times GATG$$

The short forward telomere TTAGGG largely derived from telomere G-tail and, together with the Telo_null, contributed to 91% the variation of Telephone. Referring to graph 360 of FIG. 3F, the distributions of the four Telephone markers, and two telomere markers that did not survive the LASSO modeling (TeloRv and TeloRv_null) by disease status (control, pre-HCC, HCC) and fragment size, were further dissected in Discovery and Validation phases. Consistent with the observations in Telephone modelling, HCC-associated markers shown increasing abundance in the pre-HCC blood samples collected at the intervals of 4 or more years, 3-4 years, 2-3 years, 1-2 years, and within 1 year before diagnosis (all P-trend <0.001). The difference and tread were most significant when analyzing the short ccfDNA group and to lesser extends, albeit remained statistically significant, among median- and long-ccfDNA groups. Interestingly, no differences among control, pre-HCC and HCC groups were observed for telomere reverse complement sequences TeloRv or TeloRv_null.

TABLE 5
Proportion of 260 telomere and pp4 features in short ccfDNA between Non-HCC and HCC in discovery phase.

| | | | | | Fold | | | Select |
|---|---|---|---|---|---|---|---|---|
| | | | | | change | | P | features |
| | Mean | IQR | Mean | IQR | (HCC vs | P | (Bonferron- | for model |
| Features | (Non-HCC) | (Non-HCC) | (HCC) | (HCC) | NonHCC) | (Wilcoxon) | correction) | traning |
| Telo | 0.000005403 | 0.000001143- | 0.000101949 | 0.000060232- | 18.87 | 6.40E−18 | 1.66E−15 | Yes |
| | | 0.000006869 | | 0.000130565 | | | | |
| AAAC_R2_60 | 0.001890017 | 0.001471924- | 0.003559420 | 0.003339836- | 1.88 | 6.41E−18 | 1.67E−15 | No |
| | | 0.002221428 | | 0.003764191 | | | | |
| GATG_R2_60 | 0.001345526 | 0.001060449- | 0.002805571 | 0.002525302- | 2.09 | 6.41E−18 | 1.67E−15 | Yes |
| | | 0.001597655 | | 0.003027803 | | | | |
| AAGC_R2_60 | 0.002226105 | 0.001866074- | 0.003648562 | 0.003379014- | 1.64 | 7.59E−18 | 1.97E−15 | No |
| | | 0.002483553 | | 0.003863403 | | | | |
| CAGA_R2_60 | 0.004942465 | 0.003952646- | 0.009603410 | 0.009093268- | 1.94 | 8.03E−18 | 2.09E−15 | No |
| | | 0.006003592 | | 0.010143608 | | | | |
| GAAG_R2_60 | 0.001364691 | 0.001025907- | 0.002458296 | 0.002256219- | 1.80 | 8.03E−18 | 2.09E−15 | No |
| | | 0.001642362 | | 0.002658351 | | | | |
| TCCA_R2_60 | 0.013418178 | 0.011456828- | 0.006956746 | 0.006602039- | 0.52 | 8.49E−18 | 2.21E−15 | No |
| | | 0.014851361 | | 0.007138188 | | | | |
| TCCC_R2_60 | 0.015267061 | 0.013367118- | 0.008361592 | 0.007803488- | 0.55 | 8.49E−18 | 2.21E−15 | No |
| | | 0.016964798 | | 0.009002543 | | | | |
| GAAA_R2_60 | 0.001788419 | 0.001311605- | 0.003491395 | 0.003262046- | 1.95 | 8.98E−18 | 2.34E−15 | No |
| | | 0.002145169 | | 0.003734837 | | | | |
| CAAA_R2_60 | 0.004507664 | 0.003336713- | 0.009745795 | 0.009366246- | 2.16 | 9.50E−18 | 2.47E−15 | Yes |
| | | 0.005721428 | | 0.010377232 | | | | |
| CAAG_R2_60 | 0.003094676 | 0.002419645- | 0.005777292 | 0.005541029- | 1.87 | 1.06E−17 | 2.76E−15 | No |
| | | 0.003676494 | | 0.006060152 | | | | |
| GAGA_R2_60 | 0.002124316 | 0.001681263- | 0.003869012 | 0.003456179- | 1.82 | 1.06E−17 | 2.76E−15 | No |
| | | 0.002550391 | | 0.004162868 | | | | |
| AATG_R2_60 | 0.002427425 | 0.002117364- | 0.003922033 | 0.003743291- | 1.62 | 1.26E−17 | 3.27E−15 | No |
| | | 0.002631035 | | 0.004119013 | | | | |
| CATA_R2_60 | 0.004271010 | 0.003160537- | 0.009204811 | 0.008605394- | 2.16 | 1.40E−17 | 3.65E−15 | Yes |
| | | 0.005367023 | | 0.009897978 | | | | |
| GCCA_R2_60 | 0.007100641 | 0.006312380- | 0.004404872 | 0.004135844- | 0.62 | 1.48E−17 | 3.86E−15 | No |
| | | 0.007694704 | | 0.004626189 | | | | |
| TAAA_R2_60 | 0.005097003 | 0.004534945- | 0.008385537 | 0.008014163- | 1.65 | 1.66E−17 | 4.31E−15 | No |
| | | 0.005947807 | | 0.008787081 | | | | |
| Telo_null | 0.499290706 | 0.499195711- | 0.497882784 | 0.497425079- | 0.997 | 1.66E−17 | 4.31E−15 | Yes |
| | | 0.499536086 | | 0.498377865 | | | | |
| GATA_R2_60 | 0.001342843 | 0.001034487- | 0.002792080 | 0.002624189- | 2.08 | 1.85E−17 | 4.82E−15 | Yes |
| | | 0.001645294 | | 0.002957962 | | | | |
| CATT_R2_60 | 0.002743865 | 0.002236221- | 0.004374920 | 0.004173807- | 1.59 | 2.45E−17 | 6.36E−15 | No |
| | | 0.003225763 | | 0.004617757 | | | | |
| CATG_R2_60 | 0.004676537 | 0.003464332- | 0.009802243 | 0.009172200- | 2.10 | 3.80E−17 | 9.88E−15 | Yes |
| | | 0.005623889 | | 0.010472857 | | | | |
| TATA_R2_60 | 0.004522376 | 0.003973158- | 0.008089041 | 0.007633959- | 1.79 | 5.00E−17 | 1.30E−14 | No |
| | | 0.005182291 | | 0.008553884 | | | | |
| TCCT_R2_60 | 0.013619990 | 0.010072843- | 0.004851316 | 0.004323140- | 0.36 | 5.00E−17 | 1.30E−14 | Yes |
| | | 0.016217868 | | 0.005114519 | | | | |
| CAAT_R2_60 | 0.001789049 | 0.001408469- | 0.003006784 | 0.002839043- | 1.68 | 5.58E−17 | 1.45E−14 | No |
| | | 0.002062381 | | 0.003157236 | | | | |
| ACCC_R2_60 | 0.006424722 | 0.005379450- | 0.003060637 | 0.002793837- | 0.48 | 1.48E−16 | 3.86E−14 | Yes |
| | | 0.007412331 | | 0.003260215 | | | | |
| TCGG_R2_60 | 0.000970178 | 0.000832422- | 0.000501545 | 0.000447117- | 0.52 | 1.48E−16 | 3.86E−14 | No |
| | | 0.001112240 | | 0.000536749 | | | | |
| ATTA_R2_60 | 0.002525382 | 0.002072588- | 0.004385630 | 0.004041170- | 1.74 | 1.74E−16 | 4.53E−14 | No |
| | | 0.002962611 | | 0.004791453 | | | | |
| GAGG_R2_60 | 0.001997847 | 0.001558146- | 0.003573479 | 0.003249675- | 1.79 | 1.94E−16 | 5.05E−14 | No |
| | | 0.002414320 | | 0.003913814 | | | | |
| TAGA_R2_60 | 0.003858764 | 0.003402438- | 0.005986578 | 0.005658209- | 1.55 | 2.05E−16 | 5.32E−14 | No |
| | | 0.004262508 | | 0.006227140 | | | | |
| AATA_R2_60 | 0.003377157 | 0.002894796- | 0.005483129 | 0.005308144- | 1.62 | 2.16E−16 | 5.62E−14 | No |
| | | 0.003868860 | | 0.005646437 | | | | |
| AATC_R2_60 | 0.001476277 | 0.001280145- | 0.002230406 | 0.002085741- | 1.51 | 2.68E−16 | 6.96E−14 | No |
| | | 0.001613405 | | 0.002399387 | | | | |
| GATC_R2_60 | 0.000757570 | 0.000531489- | 0.001308655 | 0.001215578- | 1.73 | 2.82E−16 | 7.34E−14 | No |
| | | 0.000928598 | | 0.001394370 | | | | |
| CGTG_R2_60 | 0.000646188 | 0.000533771- | 0.001037761 | 0.000950054- | 1.61 | 3.14E−16 | 8.17E−14 | No |
| | | 0.000767801 | | 0.001146897 | | | | |
| TGGG_R2_60 | 0.005735286 | 0.005108657- | 0.004123968 | 0.003874312- | 0.72 | 4.10E−16 | 1.07E−13 | No |
| | | 0.006347150 | | 0.004275460 | | | | |
| TATG_R2_60 | 0.003280119 | 0.002506457- | 0.005730553 | 0.005537079- | 1.75 | 4.81E−16 | 1.25E−13 | No |
| | | 0.003804427 | | 0.006017975 | | | | |
| GAGC_R2_60 | 0.001485092 | 0.001211035- | 0.002660244 | 0.002459077- | 1.79 | 5.07E−16 | 1.32E−13 | No |
| | | 0.001682550 | | 0.002884251 | | | | |
| TATC_R2_0 | 0.002345512 | 0.001949106- | 0.003703861 | 0.003485608- | 1.58 | 5.07E−16 | 1.32E−13 | No |
| | | 0.002698099 | | 0.003871041 | | | | |
| CAGG_R2_60 | 0.005588473 | 0.004379966- | 0.010391265 | 0.009887974- | 1.86 | 5.35E−16 | 1.39E−13 | No |
| | | 0.006753365 | | 0.010961704 | | | | |
| ACGC_R2_60 | 0.000959452 | 0.000740738- | 0.000411041 | 0.000350485- | 0.43 | 5.94E−16 | 1.55E−13 | Yes |
| | | 0.001209076 | | 0.000454090 | | | | |
| CAGT_R2_60 | 0.002987528 | 0.002641184- | 0.004129956 | 0.003853322- | 1.38 | 6.96E−16 | 1.81E−13 | No |
| | | 0.003269402 | | 0.004316585 | | | | |
| TCGT_R2_60 | 0.000836836 | 0.000719155- | 0.000350976 | 0.000303230- | 0.42 | 8.16E−16 | 2.12E−13 | Yes |
| | | 0.000960679 | | 0.000389076 | | | | |
| CATC_R2_60 | 0.003833119 | 0.002809591- | 0.006801022 | 0.006564573- | 1.77 | 8.60E−16 | 2.24E−13 | No |
| | | 0.004703996 | | 0.007188252 | | | | |
| TCGC_R2_60 | 0.001171490 | 0.000880960- | 0.000604988 | 0.000557608- | 0.52 | 8.60E−16 | 2.24E−13 | No |
| | | 0.001367038 | | 0.000643009 | | | | |
| GCGG_R2_60 | 0.000763353 | 0.000555519- | 0.000372174 | 0.000327714- | 0.49 | 9.06E−16 | 2.36E−13 | Yes |
| | | 0.000931053 | | 0.000401929 | | | | |
| GCCT_R2_60 | 0.004421134 | 0.003855272- | 0.002875681 | 0.002707558- | 0.65 | 9.55E−16 | 2.48E−13 | No |
| | | 0.004923633 | | 0.002985342 | | | | |
| CAGC_R2_60 | 0.005695186 | 0.004407535- | 0.011297552 | 0.010709599- | 1.98 | 1.06E−15 | 2.76E−13 | No |
| | | 0.006691329 | | 0.011932260 | | | | |
| CAAC_R2_60 | 0.003214836 | 0.002343857- | 0.006063193 | 0.005711160- | 1.89 | 1.31E−15 | 3.40E−13 | No |
| | | 0.003724046 | | 0.006519091 | | | | |
| CCTG_R2_60 | 0.006550376 | 0.004982904- | 0.011675415 | 0.010116802- | 1.78 | 2.09E−15 | 5.43E−13 | No |
| | | 0.007991425 | | 0.013109388 | | | | |
| GCGT_R2_60 | 0.000577597 | 0.000408586- | 0.000242476 | 0.000212785- | 0.42 | 2.09E−15 | 5.43E−13 | Yes |
| | | 0.000748741 | | 0.000266473 | | | | |
| TAAG_R2_60 | 0.002617045 | 0.002194986- | 0.004261072 | 0.004069839- | 1.63 | 2.20E−15 | 5.72E−13 | No |
| | | 0.003021812 | | 0.004419161 | | | | |
| GAAT_R2_60 | 0.000861843 | 0.000693709- | 0.001339280 | 0.001252435- | 1.55 | 2.44E−15 | 6.34E−13 | No |
| | | 0.001017715 | | 0.001396262 | | | | |
| CACT_R2_60 | 0.003821163 | 0.003268572- | 0.005239752 | 0.004953776- | 1.37 | 2.85E−15 | 7.41E−13 | No |
| | | 0.004455482 | | 0.005479209 | | | | |
| CCTA_R2_60 | 0.004418632 | 0.003233259- | 0.007905568 | 0.006853399- | 1.79 | 4.53E−15 | 1.18E−12 | No |
| | | 0.005385198 | | 0.009217153 | | | | |
| GAAC_R2_60 | 0.001136958 | 0.000805700- | 0.001859658 | 0.001706810- | 1.64 | 4.53E−15 | 1.18E−12 | No |
| | | 0.001461178 | | 0.001957863 | | | | |
| AAGG_R2_60 | 0.002943510 | 0.002505244- | 0.004279391 | 0.003927380- | 1.45 | 6.82E−15 | 1.77E−12 | No |
| | | 0.003483979 | | 0.004529439 | | | | |
| TAGC_R2_60 | 0.002882500 | 0.002325187- | 0.004774849 | 0.004550630- | 1.66 | 8.79E−15 | 2.29E−12 | No |
| | | 0.003203812 | | 0.005065316 | | | | |
| TAGG_R2_60 | 0.003244851 | 0.002757312- | 0.004748280 | 0.004488696- | 1.46 | 8.79E−15 | 2.29E−12 | No |
| | | 0.003617105 | | 0.004911405 | | | | |
| TTGT_R2_60 | 0.005347721 | 0.004677777- | 0.003512047 | 0.003145341- | 0.66 | 1.08E−14 | 2.80E−12 | No |
| | | 0.005912859 | | 0.003712641 | | | | |
| TAAT_R2_60 | 0.002211425 | 0.002063830- | 0.003004341 | 0.002817354- | 1.36 | 1.13E−14 | 2.94E−12 | No |
| | | 0.002437780 | | 0.003137561 | | | | |
| TAAC_R2_60 | 0.002874763 | 0.002288961- | 0.004612748 | 0.004271078- | 1.60 | 1.32E−14 | 3.42E−12 | No |
| | | 0.003185955 | | 0.004917247 | | | | |
| GACA_R2_60 | 0.002513041 | 0.002232640- | 0.003571941 | 0.003324151- | 1.42 | 1.39E−14 | 3.60E−12 | No |
| | | 0.002906874 | | 0.003764575 | | | | |
| ATTG_R2_60 | 0.001825290 | 0.001467643- | 0.002920525 | 0.002672232- | 1.60 | 1.61E−14 | 4.19E−12 | No |
| | | 0.002177197 | | 0.003254012 | | | | |
| ACGG_R2_60 | 0.001263342 | 0.001028151- | 0.000433870 | 0.000356389- | 0.34 | 2.18E−14 | 5.66E−12 | Yes |
| | | 0.001576576 | | 0.000480347 | | | | |
| CGTA_R2_60 | 0.000377488 | 0.000291735- | 0.000682833 | 0.000602557- | 1.81 | 2.29E−14 | 5.95E−12 | No |
| | | 0.000424410 | | 0.000765954 | | | | |
| GACC_R2_60 | 0.001473945 | 0.001104938- | 0.002701130 | 0.002446656- | 1.83 | 2.66E−14 | 6.91E−12 | No |
| | | 0.001659679 | | 0.002954224 | | | | |
| ACAC_R2_60 | 0.005236301 | 0.003644281- | 0.002737392 | 0.002541534- | 0.52 | 3.09E−14 | 8.02E−12 | No |
| | | 0.006870099 | | 0.002858400 | | | | |
| ACCA_R2_60 | 0.010587676 | 0.008474819- | 0.004689809 | 0.004063835- | 0.44 | 3.24E−14 | 8.43E−12 | Yes |
| | | 0.013365042 | | 0.004949487 | | | | |
| TGCA_R2_60 | 0.006673885 | 0.005568238- | 0.004457105 | 0.004180454- | 0.67 | 3.24E−14 | 8.43E−12 | No |
| | | 0.007749570 | | 0.004600980 | | | | |
| TeloRv_null | 0.498431173 | 0.498142013- | 0.496562423 | 0.495891760- | 0.996 | 3.41E−14 | 8.86E−12 | Yes |
| | | 0.499007917 | | 0.497165834 | | | | |
| GTGT_R2_60 | 0.004004106 | 0.002850109- | 0.002172540 | 0.002000417- | 0.54 | 3.95E−14 | 1.03E−11 | No |
| | | 0.005244966 | | 0.002293473 | | | | |
| GACG_R2_60 | 0.000241522 | 0.000160134- | 0.000411885 | 0.000361910- | 1.71 | 4.82E−14 | 1.25E−11 | No |
| | | 0.000299782 | | 0.000462828 | | | | |
| GCGA_R2_60 | 0.000653496 | 0.000471606- | 0.000275457 | 0.000239646- | 0.42 | 5.59E−14 | 1.45E−11 | Yes |
| | | 0.000834226 | | 0.000295913 | | | | |
| ACAG_R2_60 | 0.004001436 | 0.003485738- | 0.002553685 | 0.002303174- | 0.64 | 7.88E−14 | 2.05E−11 | No |
| | | 0.004662577 | | 0.002688599 | | | | |
| TCAG_R2_60 | 0.005131660 | 0.004593060- | 0.003883295 | 0.003710328- | 0.76 | 7.88E−14 | 2.05E−11 | No |
| | | 0.005919632 | | 0.004007888 | | | | |
| TTTA_R2_60 | 0.006341018 | 0.004908617- | 0.010207676 | 0.009333324- | 1.61 | 7.88E−14 | 2.05E−11 | No |
| | | 0.007691435 | | 0.011306593 | | | | |
| CACA_R2_60 | 0.008577905 | 0.006921688- | 0.012277641 | 0.011683460- | 1.43 | 8.69E−14 | 2.26E−11 | No |
| | | 0.009647219 | | 0.012992149 | | | | |
| CTTA_R2_60 | 0.006032063 | 0.004331660- | 0.010124326 | 0.009606289- | 1.68 | 8.69E−14 | 2.26E−11 | No |
| | | 0.007148889 | | 0.010656578 | | | | |
| TCAT_R2_60 | 0.004266872 | 0.003607916- | 0.002709588 | 0.002416189- | 0.64 | 8.69E−14 | 2.26E−11 | No |
| | | 0.004944934 | | 0.002998965 | | | | |
| CACG_R2_60 | 0.001240371 | 0.000907596- | 0.002089057 | 0.001857077- | 1.68 | 1.01E−13 | 2.61E−11 | No |
| | | 0.001511513 | | 0.002338373 | | | | |
| TGGT_R2_60 | 0.003061411 | 0.002716749- | 0.001841144 | 0.001637775- | 0.60 | 1.11E−13 | 2.88E−11 | No |
| | | 0.003407197 | | 0.001940213 | | | | |
| GACT_R2_60 | 0.000991574 | 0.000803252- | 0.001455536 | 0.001338853- | 1.47 | 1.63E−13 | 4.25E−11 | No |
| | | 0.001151477 | | 0.001584168 | | | | |
| GTTA_R2_60 | 0.002059475 | 0.001637854- | 0.003285502 | 0.003038413- | 1.60 | 2.18E−13 | 5.67E−11 | No |
| | | 0.002258811 | | 0.003510737 | | | | |
| TGCC_R2_60 | 0.005689818 | 0.005336255- | 0.004688427 | 0.004423669- | 0.82 | 3.20E−13 | 8.33E−11 | No |
| | | 0.006223978 | | 0.004952729 | | | | |
| TGGA_R2_60 | 0.005950494 | 0.005210390- | 0.003858735 | 0.003540395- | 0.65 | 3.52E−13 | 9.16E−11 | No |
| | | 0.006672869 | | 0.003949300 | | | | |
| AAAG_R2_60 | 0.002445318 | 0.001947355- | 0.003610189 | 0.003312282- | 1.48 | 3.88E−13 | 1.01E−10 | No |
| | | 0.002973927 | | 0.003853174 | | | | |
| ACGT_R2_60 | 0.001016216 | 0.000825944- | 0.000425973 | 0.000358540- | 0.42 | 5.15E−13 | 1.34E−10 | Yes |
| | | 0.001243212 | | 0.000454148 | | | | |
| CTTG_R2_60 | 0.006342144 | 0.004789457- | 0.010263392 | 0.009632949- | 1.62 | 5.15E−13 | 1.34E−10 | No |
| | | 0.007251673 | | 0.010954992 | | | | |
| CACC_R2_60 | 0.007692580 | 0.005520574- | 0.012705260 | 0.011599265- | 1.65 | 6.22E−13 | 1.62E−10 | No |
| | | 0.008890301 | | 0.013812986 | | | | |
| AAAT_R2_60 | 0.001894568 | 0.001591299- | 0.002666871 | 0.002470599- | 1.41 | 6.53E−13 | 1.70E−10 | No |
| | | 0.002302333 | | 0.002801977 | | | | |
| GGTT_R2_60 | 0.001380636 | 0.001079629- | 0.002014755 | 0.001806094- | 1.46 | 9.50E−13 | 2.47E−10 | No |
| | | 0.001588250 | | 0.002135173 | | | | |
| ACGA_R2_60 | 0.001022406 | 0.000772909- | 0.000409544 | 0.000324321- | 0.40 | 1.20E−12 | 3.12E−10 | Yes |
| | | 0.001300952 | | 0.000430284 | | | | |
| GGGA_R2_60 | 0.006772782 | 0.005944546- | 0.004478484 | 0.004125135- | 0.66 | 1.45E−12 | 3.76E−10 | No |
| | | 0.007741049 | | 0.004671533 | | | | |
| AAAA_R2_60 | 0.004189869 | 0.003215545- | 0.006285600 | 0.005850819- | 1.50 | 2.30E−12 | 5.97E−10 | No |
| | | 0.004934475 | | 0.006445044 | | | | |
| AACC_R2_60 | 0.002574134 | 0.002045536- | 0.003613032 | 0.003327510- | 1.40 | 3.03E−12 | 7.87E−10 | No |
| | | 0.002972381 | | 0.003891685 | | | | |
| CGGG_R2_60 | 0.000549359 | 0.000452880- | 0.000794127 | 0.000736680- | 1.45 | 3.03E−12 | 7.87E−10 | No |
| | | 0.000626972 | | 0.000852393 | | | | |
| GCTA_R2_60 | 0.002130476 | 0.001793701- | 0.003165769 | 0.002772221- | 1.49 | 3.17E−12 | 8.23E−10 | No |
| | | 0.002371854 | | 0.003566732 | | | | |
| GTTG_R2_60 | 0.001999935 | 0.001719083- | 0.002960437 | 0.002690238- | 1.48 | 3.17E−12 | 8.23E−10 | No |
| | | 0.002137364 | | 0.003175790 | | | | |
| TACC_R2_60 | 0.003869341 | 0.003234182- | 0.005553678 | 0.005078197- | 1.44 | 3.98E−12 | 1.03E−09 | No |
| | | 0.004309177 | | 0.005934024 | | | | |
| TTCT_R2_60 | 0.010146551 | 0.007986930- | 0.006113226 | 0.005743464- | 0.60 | 4.16E−12 | 1.08E−09 | No |
| | | 0.012326003 | | 0.006572922 | | | | |
| GCTG_R2_60 | 0.002965020 | 0.002433391- | 0.004314752 | 0.003844436- | 1.46 | 4.77E−12 | 1.24E−09 | No |
| | | 0.003412707 | | 0.004701283 | | | | |
| ACCT_R2_60 | 0.005916362 | 0.004395663- | 0.002804890 | 0.002416369- | 0.47 | 5.72E−12 | 1.49E−09 | Yes |
| | | 0.007657457 | | 0.002896842 | | | | |
| GCCC_R2_60 | 0.005710941 | 0.004276662- | 0.003902994 | 0.003704313- | 0.68 | 6.86E−12 | 1.78E−09 | No |
| | | 0.006550973 | | 0.004132462 | | | | |
| TCGA_R2_60 | 0.001009718 | 0.000873455- | 0.000525754 | 0.000487515- | 0.52 | 8.21E−12 | 2.13E−09 | No |
| | | 0.001186536 | | 0.000557847 | | | | |
| ACAA_R2_60 | 0.005018714 | 0.004220213- | 0.003278092 | 0.002971217- | 0.65 | 8.98E−12 | 2.33E−09 | No |
| | | 0.005864422 | | 0.003493703 | | | | |
| GATT_R2_60 | 0.000816088 | 0.000684940- | 0.001135475 | 0.001058696- | 1.39 | 9.39E−12 | 2.44E−09 | No |
| | | 0.000942517 | | 0.001198900 | | | | |
| TGAG_R2_60 | 0.003918991 | 0.003548834- | 0.003036874 | 0.002812457- | 0.77 | 1.40E−11 | 3.65E−09 | No |
| | | 0.004167749 | | 0.003227171 | | | | |
| CGGC_R2_60 | 0.000463955 | 0.000339517- | 0.000707526 | 0.000651355- | 1.52 | 1.75E−11 | 4.55E−09 | No |
| | | 0.000561974 | | 0.000755285 | | | | |
| TCAA_R2_60 | 0.005553824 | 0.005258961- | 0.004628508 | 0.004249616- | 0.83 | 2.60E−11 | 6.77E−09 | No |
| | | 0.005907179 | | 0.005003224 | | | | |
| GCGC_R2_60 | 0.000864882 | 0.000440671- | 0.000373170 | 0.000333905- | 0.43 | 3.69E−11 | 9.60E−09 | Yes |
| | | 0.001126989 | | 0.000412242 | | | | |
| AGGT_R2_60 | 0.003915875 | 0.002907191- | 0.002276046 | 0.001873900- | 0.58 | 4.79E−11 | 1.25E−08 | No |
| | | 0.004957796 | | 0.002437183 | | | | |
| TTAT_R2_60 | 0.003964124 | 0.003513988- | 0.003027660 | 0.002701435- | 0.76 | 5.45E−11 | 1.42E−08 | No |
| | | 0.004370765 | | 0.003284751 | | | | |
| TTTG_R2_60 | 0.005883012 | 0.004379797- | 0.008653063 | 0.008203247- | 1.47 | 5.45E−11 | 1.42E−08 | No |
| | | 0.006839531 | | 0.009299737 | | | | |
| GGGT_R2_60 | 0.002849087 | 0.002391345- | 0.001830179 | 0.001678746- | 0.64 | 7.06E−11 | 1.84E−08 | No |
| | | 0.003407883 | | 0.001937867 | | | | |
| TCTA_R2_60 | 0.004697394 | 0.003935287- | 0.006290488 | 0.005776218- | 1.34 | 2.22E−10 | 5.76E−08 | No |
| | | 0.005314048 | | 0.006790256 | | | | |
| AGGG_R2_60 | 0.006254042 | 0.004699789- | 0.003694197 | 0.003119982- | 0.59 | 2.97E−10 | 7.72E08 | No |
| | | 0.007761958 | | 0.004071686 | | | | |
| TCAC_R2_60 | 0.007969299 | 0.004774936- | 0.004146164 | 0.003947522- | 0.52 | 3.09E−10 | 8.04E−08 | No |
| | | 0.010285771 | | 0.004391410 | | | | |
| CTCA_R2_60 | 0.012688564 | 0.011659850- | 0.014267651 | 0.013961935- | 1.12 | 3.36E−10 | 8.74E−08 | No |
| | | 0.013116962 | | 0.014754692 | | | | |
| AGGC_R2_60 | 0.004835930 | 0.004193730- | 0.003319917 | 0.002944390- | 0.69 | 4.31E−10 | 1.12E−07 | No |
| | | 0.005607834 | | 0.003534979 | | | | |
| GAGT_R2_60 | 0.001042331 | 0.000908122- | 0.001348359 | 0.001265551- | 1.29 | 4.87E−10 | 1.27E−07 | No |
| | | 0.001168202 | | 0.001431822 | | | | |
| TGCT_R2_60 | 0.003830346 | 0.003217520- | 0.002583886 | 0.002388759- | 0.67 | 4.87E−10 | 1.27E−07 | No |
| | | 0.004444091 | | 0.002712234 | | | | |
| CCAA_R2_60 | 0.005090179 | 0.004287584- | 0.006364901 | 0.005830566- | 1.25 | 5.74E−10 | 1.49E−07 | No |
| | | 0.005631199 | | 0.006797729 | | | | |
| TACG_R2_60 | 0.000521014 | 0.000402343- | 0.000761660 | 0.000703531- | 1.46 | 6.49E−10 | 1.69E−07 | No |
| | | 0.000680961 | | 0.000815874 | | | | |
| CGCC_R2_60 | 0.000854942 | 0.000683051- | 0.001104898 | 0.001016465- | 1.29 | 1.78E−09 | 4.62E−07 | No |
| | | 0.000981957 | | 0.001185002 | | | | |
| CTGT_R2_60 | 0.006445889 | 0.005858873- | 0.005416842 | 0.005181194- | 0.84 | 1.78E−09 | 4.62E−07 | No |
| | | 0.006832975 | | 0.005551073 | | | | |
| TCCG_R2_60 | 0.001110460 | 0.000938968- | 0.000835866 | 0.000775595- | 0.75 | 1.85E−09 | 4.80E−07 | No |
| | | 0.001249401 | | 0.000893486 | | | | |
| CCCT_R2_60 | 0.007679865 | 0.006389768- | 0.005862918 | 0.005522340- | 0.76 | 2.34E−09 | 6.09E−07 | No |
| | | 0.008200099 | | 0.006112693 | | | | |
| AGCA_R2_60 | 0.010414096 | 0.007156626- | 0.005834942 | 0.004668894- | 0.56 | 2.64E−09 | 6.86E−07 | No |
| | | 0.013526269 | | 0.006413811 | | | | |
| ATTC_R2_60 | 0.002239561 | 0.001858699- | 0.003022726 | 0.002693453- | 1.35 | 2.85E−09 | 7.42E−07 | No |
| | | 0.002636967 | | 0.003309339 | | | | |
| CCCG_R2_60 | 0.001531247 | 0.001296904- | 0.002024508 | 0.001799837- | 1.32 | 2.85E−09 | 7.42E−07 | No |
| | | 0.001735908 | | 0.002222845 | | | | |
| TTAG_R2_60 | 0.003414217 | 0.002989655- | 0.002724751 | 0.002492378- | 0.80 | 2.85E−09 | 7.42E−07 | No |
| | | 0.003878940 | | 0.002948538 | | | | |
| ATTT_R2_60 | 0.002295584 | 0.001638252- | 0.003192115 | 0.002856607- | 1.39 | 3.09E−09 | 8.02E−07 | No |
| | | 0.002752549 | | 0.003549596 | | | | |
| ATCG_R2_60 | 0.000274417 | 0.000236237- | 0.000394199 | 0.000333616- | 1.44 | 3.34E−09 | 8.67E−07 | No |
| | | 0.000324175 | | 0.000442103 | | | | |
| CTCG_R2_60 | 0.001321188 | 0.001040169- | 0.001795645 | 0.001652925- | 1.36 | 3.34E−09 | 8.67E−07 | No |
| | | 0.001577734 | | 0.001965618 | | | | |
| AGCG_R2_60 | 0.000952806 | 0.000696227- | 0.000516218 | 0.000409562- | 0.54 | 3.61E−09 | 9.38E−07 | No |
| | | 0.001209287 | | 0.000572573 | | | | |
| ACCG_R2_60 | 0.000872192 | 0.000613136- | 0.000422800 | 0.000355853- | 0.48 | 4.56E−09 | 1.18E−06 | Yes |
| | | 0.001153513 | | 0.000452002 | | | | |
| AGTG_R2_60 | 0.004849948 | 0.003838323- | 0.003396675 | 0.003187647- | 0.70 | 6.45E−09 | 1.68E−06 | No |
| | | 0.005804202 | | 0.003513926 | | | | |
| TCTG_R2_60 | 0.005776912 | 0.004746518- | 0.007297568 | 0.006873852- | 1.26 | 7.24E−09 | 1.88E−06 | No |
| | | 0.006522940 | | 0.007749822 | | | | |
| AAGA_R2_60 | 0.004070466 | 0.003121221- | 0.005462467 | 0.005115608- | 1.34 | 1.33E−08 | 3.45E−06 | No |
| | | 0.004996350 | | 0.005671706 | | | | |
| CTCT_R2_60 | 0.013019206 | 0.010613707- | 0.009168884 | 0.008787724- | 0.70 | 2.08E−08 | 5.41E−06 | No |
| | | 0.015765363 | | 0.009517833 | | | | |
| TTGA_R2_60 | 0.006161037 | 0.005575714- | 0.005159980 | 0.004730327- | 0.84 | 2.08E−08 | 5.41E−06 | No |
| | | 0.006801886 | | 0.005446020 | | | | |
| ACAT_R2_60 | 0.003157467 | 0.002854405- | 0.002581172 | 0.002332066- | 0.82 | 2.80E−08 | 7.28E−06 | No |
| | | 0.003446024 | | 0.002820481 | | | | |
| AGAG_R2_60 | 0.005152599 | 0.003899244- | 0.003603969 | 0.003136171- | 0.70 | 3.62E−08 | 9.41E−06 | No |
| | | 0.006181886 | | 0.003915413 | | | | |
| GGGG_R2_60 | 0.004281755 | 0.003525022- | 0.003257740 | 0.003083871- | 0.76 | 3.89E−08 | 1.01E−05 | No |
| | | 0.004885292 | | 0.003417773 | | | | |
| TTCG_R2_60 | 0.000746238 | 0.000607699- | 0.000947756 | 0.000881612- | 1.27 | 4.19E−08 | 1.09E−05 | No |
| | | 0.000884548 | | 0.001016159 | | | | |
| AGCC_R2_60 | 0.005609020 | 0.004361829- | 0.004052565 | 0.003547426- | 0.72 | 4.67E−08 | 1.21E−05 | No |
| | | 0.006531233 | | 0.004398854 | | | | |
| AGGA_R2_60 | 0.010398959 | 0.006690510- | 0.005683756 | 0.004409812- | 0.55 | 4.67E−08 | 1.21E−05 | No |
| | | 0.014728896 | | 0.006296481 | | | | |
| CTGG_R2_60 | 0.008020514 | 0.007177661- | 0.009170657 | 0.008841677- | 1.14 | 6.70E−08 | 1.74E−05 | No |
| | | 0.008241735 | | 0.009521422 | | | | |
| GTTC_R2_60 | 0.001965248 | 0.001639080- | 0.002419989 | 0.002254044- | 1.23 | 6.95E−08 | 1.81E−05 | No |
| | | 0.002267095 | | 0.002578731 | | | | |
| TTCC_R2_60 | 0.015169259 | 0.013408569- | 0.012253905 | 0.011166437- | 0.81 | 1.06E−07 | 2.77E−05 | No |
| | | 0.016283301 | | 0.013404583 | | | | |
| CGTC_R2_60 | 0.000436839 | 0.000344079- | 0.000558829 | 0.000513446- | 1.28 | 1.10E−07 | 2.87E−05 | No |
| | | 0.000514650 | | 0.000605569 | | | | |
| CGGA_R2_60 | 0.000332464 | 0.000268356- | 0.000436501 | 0.000401361- | 1.31 | 1.62E−07 | 4.22E−05 | No |
| | | 0.000395941 | | 0.000480916 | | | | |
| CCCA_R2_60 | 0.013259567 | 0.011817628- | 0.011517540 | 0.010542098- | 0.87 | 2.63E−07 | 6.85E−05 | No |
| | | 0.014295183 | | 0.012385900 | | | | |
| GCAC_R2_60 | 0.004374718 | 0.002084114- | 0.001958138 | 0.001843889- | 0.45 | 2.63E−07 | 6.85E−05 | Yes |
| | | 0.006144615 | | 0.002026703 | | | | |
| GCTC_R2_60 | 0.002125505 | 0.001727752- | 0.002604886 | 0.002403834- | 1.23 | 2.92E−07 | 7.59E−05 | No |
| | | 0.002395456 | | 0.002818549 | | | | |
| GCTT_R2_60 | 0.001710988 | 0.001304964- | 0.002175670 | 0.001965001- | 1.27 | 3.71E−07 | 9.63E−05 | No |
| | | 0.001971400 | | 0.002402342 | | | | |
| TGTA_R2_60 | 0.004689113 | 0.004130185- | 0.005559271 | 0.004988287- | 1.19 | 5.02E−07 | 1.31E−04 | No |
| | | 0.005224807 | | 0.006080201 | | | | |
| TGGC_R2_60 | 0.004445937 | 0.003706666- | 0.003619075 | 0.003413371- | 0.81 | 8.00E−07 | 2.08E−04 | No |
| | | 0.005255652 | | 0.003812590 | | | | |
| AACG_R2_60 | 0.000371831 | 0.000300362- | 0.000483660 | 0.000428490- | 1.30 | 8.27E−07 | 2.15E−04 | No |
| | | 0.000428106 | | 0.000535974 | | | | |
| CTAA_R2_60 | 0.005600207 | 0.005135412- | 0.006269632 | 0.006015739- | 1.12 | 1.04E−06 | 2.70E−04 | No |
| | | 0.005953157 | | 0.006590088 | | | | |
| TGAA_R2_60 | 0.004370686 | 0.004096028- | 0.003771047 | 0.003466457- | 0.86 | 1.11E−06 | 2.89E−04 | No |
| | | 0.004749487 | | 0.004027564 | | | | |
| GTAC_R2_60 | 0.003281645 | 0.001654788- | 0.001557873 | 0.001359133- | 0.47 | 1.22E−06 | 3.18E−04 | Yes |
| | | 0.004450960 | | 0.001694657 | | | | |
| GTTT_R2_60 | 0.001918788 | 0.001386513- | 0.002469395 | 0.002308276- | 1.29 | 1.26E−06 | 3.29E−04 | No |
| | | 0.002395526 | | 0.002599251 | | | | |
| ATGA_R2_60 | 0.003681980 | 0.003165503- | 0.003035871 | 0.002822559- | 0.82 | 1.35E−06 | 3.51E−04 | No |
| | | 0.004144227 | | 0.003282364 | | | | |
| CCGT_R2_60 | 0.000783485 | 0.000611439- | 0.000591690 | 0.000521766- | 0.76 | 1.80E−06 | 4.69E−04 | No |
| | | 0.000938171 | | 0.000642250 | | | | |
| TGCG_R2_60 | 0.000661644 | 0.000530133- | 0.000531970 | 0.000484980- | 0.80 | 2.99E−06 | 7.78E−04 | No |
| | | 0.000770333 | | 0.000572388 | | | | |
| GTCG_R2_60 | 0.000290106 | 0.000210229- | 0.000365656 | 0.000328472- | 1.26 | 4.91E−06 | 0.001277824 | No |
| | | 0.000333963 | | 0.000400849 | | | | |
| CGAC_R2_60 | 0.000266449 | 0.000217630- | 0.000336631 | 0.000303324- | 1.26 | 6.09E−06 | 0.001582776 | No |
| | | 0.000317369 | | 0.000363281 | | | | |
| GTAG_R2_60 | 0.002490727 | 0.001511415- | 0.001540899 | 0.001411320- | 0.62 | 6.87E−06 | 0.001787113 | No |
| | | 0.003081970 | | 0.001656216 | | | | |
| GCAT_R2_60 | 0.002539808 | 0.001776355- | 0.001727483 | 0.001585916- | 0.68 | 7.99E−06 | 0.002078157 | No |
| | | 0.002967337 | | 0.001848223 | | | | |
| ATGT_R2_60 | 0.002994944 | 0.002718618- | 0.002571404 | 0.002334823- | 0.86 | 8.24E−06 | 0.002141567 | No |
| | | 0.003316319 | | 0.002898817 | | | | |
| AGTC_R2_60 | 0.002527183 | 0.002126440- | 0.002100388 | 0.001972234- | 0.83 | 9.01E−06 | 0.002343084 | No |
| | | 0.002936089 | | 0.002180229 | | | | |
| GTGA_R2_60 | 0.005079768 | 0.002946597- | 0.002854093 | 0.002604388- | 0.56 | 1.11E−05 | 0.002886118 | No |
| | | 0.006672853 | | 0.003076538 | | | | |
| CGAA_R2_60 | 0.000337401 | 0.000230769- | 0.000428657 | 0.000372438- | 1.27 | 1.21E−05 | 0.003153929 | No |
| | | 0.000391100 | | 0.000462294 | | | | |
| GTCT_R2_60 | 0.003577265 | 0.002938920- | 0.002946914 | 0.002786729- | 0.82 | 1.21E−05 | 0.003153929 | No |
| | | 0.004061996 | | 0.003128562 | | | | |
| AGCT_R2_60 | 0.004708186 | 0.003155585- | 0.003018024 | 0.002518222- | 0.64 | 1.49E−05 | 0.003874081 | No |
| | | 0.006226589 | | 0.003312364 | | | | |
| ACTA_R2_60 | 0.002349069 | 0.002067638- | 0.002715916 | 0.002486818- | 1.16 | 2.11E−05 | 0.005486756 | No |
| | | 0.002678695 | | 0.002925247 | | | | |
| CTAT_R2_60 | 0.003812598 | 0.003326449- | 0.003318535 | 0.003029436- | 0.87 | 2.65E−05 | 0.006897644 | No |
| | | 0.004326823 | | 0.003562592 | | | | |
| TTAC_R2_60 | 0.004971976 | 0.003288878- | 0.003339502 | 0.003095793- | 0.67 | 2.97E−05 | 0.007726447 | No |
| | | 0.006082871 | | 0.003598638 | | | | |
| GTAT_R2_60 | 0.002602628 | 0.001824157- | 0.001783281 | 0.001622710- | 0.69 | 3.14E−05 | 0.008175532 | No |
| | | 0.003607954 | | 0.001887317 | | | | |
| GGCA_R2_60 | 0.005758671 | 0.005016761- | 0.004951795 | 0.004683039- | 0.86 | 3.33E−05 | 0.008649348 | No |
| | | 0.006568076 | | 0.005189434 | | | | |
| GGTA_R2_60 | 0.002964959 | 0.002289875- | 0.003484887 | 0.003153416- | 1.18 | 4.91E−05 | 0.012774032 | No |
| | | 0.003447423 | | 0.003817267 | | | | |
| GTAA_R2_60 | 0.003064594 | 0.002288670- | 0.002268638 | 0.002114568- | 0.74 | 5.64E−05 | 0.014655198 | No |
| | | 0.003654644 | | 0.002378516 | | | | |
| GGCC_R2_60 | 0.003466787 | 0.003204713- | 0.003760684 | 0.003563280- | 1.08 | 5.79E−05 | 0.015061663 | No |
| | | 0.003683507 | | 0.003976238 | | | | |
| AGAA_R2_60 | 0.008272755 | 0.005373704- | 0.005688287 | 0.004692838- | 0.69 | 6.46E−05 | 0.016796781 | No |
| | | 0.011635301 | | 0.006236945 | | | | |
| CCAC_R2_60 | 0.010430943 | 0.006141242- | 0.006192075 | 0.005828462- | 0.59 | 7.81E−05 | 0.020297069 | No |
| | | 0.013713573 | | 0.006612627 | | | | |
| CGAG_R2_60 | 0.000372914 | 0.000260567- | 0.000478050 | 0.000427247- | 1.28 | 1.13E−04 | 0.029466442 | No |
| | | 0.000490950 | | 0.000530098 | | | | |
| AGAC_R2_60 | 0.003191921 | 0.002409404- | 0.002471269 | 0.002292431- | 0.77 | 1.51E−04 | 0.03928044 | No |
| | | 0.003977358 | | 0.002562194 | | | | |
| AGTA_R2_60 | 0.004298054 | 0.003698531- | 0.003710860 | 0.003434538- | 0.86 | 1.86E−04 | 0.048269864 | No |
| | | 0.005016931 | | 0.003924193 | | | | |
| CGTT_R2_60 | 0.000284927 | 0.000228300- | 0.000344451 | 0.000297683- | 1.21 | 1.95E−04 | 0.050801931 | No |
| | | 0.000336268 | | 0.000395971 | | | | |
| CGGT_R2_60 | 0.000310293 | 0.000218849- | 0.000230307 | 0.000192611- | 0.74 | 2.06E−04 | 0.053456494 | No |
| | | 0.000391343 | | 0.000266451 | | | | |
| GTGG_R2_60 | 0.005589356 | 0.003168572- | 0.003369129 | 0.003040075- | 0.60 | 3.74E−04 | 0.097348165 | No |
| | | 0.007704552 | | 0.003681330 | | | | |
| GGGC_R2_60 | 0.003488501 | 0.002519617- | 0.002633001 | 0.002515149- | 0.75 | 3.84E−04 | 0.099761113 | No |
| | | 0.004299609 | | 0.002773611 | | | | |
| TCTT_R2_60 | 0.005456897 | 0.003589524- | 0.003950439 | 0.003644557- | 0.72 | 4.13E−04 | 0.107339568 | No |
| | | 0.006955060 | | 0.004237396 | | | | |
| GCAG_R2_60 | 0.003083862 | 0.002217802- | 0.002337839 | 0.002202382- | 0.76 | 6.51E−04 | 0.169278587 | No |
| | | 0.003811757 | | 0.002470611 | | | | |
| CCTC_R2_60 | 0.007922779 | 0.005712491- | 0.009606042 | 0.008741562- | 1.21 | 7.32E−04 | 0.190391915 | No |
| | | 0.010485319 | | 0.010407089 | | | | |
| GCAA_R2_60 | 0.003123558 | 0.002425790- | 0.002558970 | 0.002368829- | 0.82 | 7.67E−04 | 0.199502612 | No |
| | | 0.003776628 | | 0.002700336 | | | | |
| ATAG_R2_60 | 0.001990515 | 0.001706932- | 0.001717118 | 0.001479732- | 0.86 | 8.62E−04 | 0.224079814 | No |
| | | 0.002235495 | | 0.001875801 | | | | |
| ATAC_R2_60 | 0.002756800 | 0.001977619- | 0.002072673 | 0.001748135- | 0.75 | 9.03E−04 | 0.234674571 | No |
| | | 0.003244925 | | 0.002327168 | | | | |
| CTAC_R2_60 | 0.006758252 | 0.004459333- | 0.004975629 | 0.004721714- | 0.74 | 1.30E−03 | 0.337707639 | No |
| | | 0.008441391 | | 0.005256144 | | | | |
| ATCC_R2_60 | 0.004717681 | 0.004183205- | 0.005238604 | 0.004573581- | 1.11 | 0.001771512 | 0.460593141 | No |
| | | 0.005173559 | | 0.005890395 | | | | |
| TCTC_R2_60 | 0.007954645 | 0.005962382- | 0.006270218 | 0.006073914- | 0.79 | 0.002248687 | 0.584658636 | No |
| | | 0.009576959 | | 0.006599236 | | | | |
| GGTC_R2_60 | 0.001777448 | 0.001300425- | 0.001996463 | 0.001928054- | 1.12 | 0.002723775 | 0.708181406 | No |
| | | 0.002088069 | | 0.002128615 | | | | |
| GGCT_R2_60 | 0.002859000 | 0.002397482- | 0.002543321 | 0.002345346- | 0.89 | 0.003221263 | 0.837528476 | No |
| | | 0.003206955 | | 0.002657157 | | | | |
| TTAA_R2_60 | 0.005867253 | 0.005333140- | 0.005435235 | 0.005010719- | 0.93 | 0.004040702 | 1 | No |
| | | 0.006423127 | | 0.005884372 | | | | |
| GCCG_R2_60 | 0.000748184 | 0.000608176- | 0.000635439 | 0.000569815- | 0.85 | 0.005463015 | 1 | No |
| | | 0.000857268 | | 0.000711840 | | | | |
| CGCG_R2_60 | 0.000189973 | 0.000132956- | 0.000207104 | 0.000179743- | 1.09 | 0.009199955 | 1 | No |
| | | 0.000232725 | | 0.000230149 | | | | |
| TATT_R2_60 | 0.003244950 | 0.002678089- | 0.003553363 | 0.003300675- | 1.10 | 0.011707926 | 1 | No |
| | | 0.003922825 | | 0.003677401 | | | | |
| AATT_R2_60 | 0.001829627 | 0.001394740- | 0.002158326 | 0.001969726- | 1.18 | 0.015070398 | 1 | No |
| | | 0.002220220 | | 0.002205125 | | | | |
| TTGG_R2_60 | 0.005384285 | 0.004958689- | 0.005018301 | 0.004682166- | 0.93 | 0.01735159 | 1 | No |
| | | 0.005675884 | | 0.005320265 | | | | |
| CCAT_R2_60 | 0.004574955 | 0.003642451- | 0.003941820 | 0.003551376- | 0.86 | 0.01765703 | 1 | No |
| | | 0.005513207 | | 0.004252199 | | | | |
| ATCT_R2_60 | 0.002951901 | 0.002740495- | 0.003149006 | 0.002903034- | 1.07 | 0.024018529 | 1 | No |
| | | 0.003260590 | | 0.003473002 | | | | |
| TGAC_R2_60 | 0.003410954 | 0.002377554- | 0.002596625 | 0.002378254- | 0.76 | 0.024018529 | 1 | No |
| | | 0.004105770 | | 0.002773433 | | | | |
| AGAT_R2_60 | 0.002561486 | 0.002273722- | 0.002407981 | 0.002153642- | 0.94 | 0.024423956 | 1 | No |
| | | 0.002904579 | | 0.002588429 | | | | |
| CCAG_R2_60 | 0.005662657 | 0.004807331- | 0.006089614 | 0.005610068- | 1.08 | 0.025252632 | 1 | No |
| | | 0.006615968 | | 0.006485722 | | | | |
| AAGT_R2_60 | 0.002368420 | 0.001977497- | 0.002227122 | 0.002029682- | 0.94 | 0.025676019 | 1 | No |
| | | 0.002702219 | | 0.002328578 | | | | |
| GGAT_R2_60 | 0.001750602 | 0.001306888- | 0.001906478 | 0.001730855- | 1.09 | 0.029289649 | 1 | No |
| | | 0.002036089 | | 0.002077425 | | | | |
| CTGC_R2_60 | 0.009797389 | 0.007873824- | 0.010317270 | 0.010005631- | 1.05 | 0.030753376 | 1 | No |
| | | 0.011299566 | | 0.010843134 | | | | |
| AGTT_R2_60 | 0.002013153 | 0.001614646- | 0.002249409 | 0.002038717- | 1.12 | 0.036669802 | 1 | No |
| | | 0.002481868 | | 0.002470241 | | | | |
| CTGA_R2_60 | 0.007520801 | 0.006325639- | 0.007955059 | 0.007529594- | 1.06 | 0.040287589 | 1 | No |
| | | 0.008556101 | | 0.008450136 | | | | |
| TAGT_R2_60 | 0.002394851 | 0.002059534- | 0.002579667 | 0.002348114- | 1.08 | 0.042207198 | 1 | No |
| | | 0.002762375 | | 0.002638644 | | | | |
| TACA_R2_60 | 0.005876638 | 0.004752234- | 0.006466913 | 0.006231657- | 1.10 | 0.050677019 | 1 | No |
| | | 0.007022061 | | 0.006650979 | | | | |
| ATAT_R2_60 | 0.002515486 | 0.002212515- | 0.002680303 | 0.002405309- | 1.07 | 0.057080551 | 1 | No |
| | | 0.002763613 | | 0.003019290 | | | | |
| CCTT_R2_60 | 0.005021504 | 0.003208678- | 0.005564848 | 0.005034275- | 1.11 | 0.059649321 | 1 | No |
| | | 0.006688686 | | 0.006200036 | | | | |
| AACA_R2_60 | 0.005284744 | 0.003818447- | 0.005695491 | 0.005230563- | 1.08 | 0.066017542 | 1 | No |
| | | 0.006566759 | | 0.005912885 | | | | |
| CGCT_R2_60 | 0.000357944 | 0.000262925- | 0.000371876 | 0.000335348- | 1.04 | 0.067936655 | 1 | No |
| | | 0.000394365 | | 0.000403670 | | | | |
| TGTG_R2_60 | 0.006377829 | 0.005356110- | 0.005895187 | 0.005621782- | 0.92 | 0.073970889 | 1 | No |
| | | 0.006958140 | | 0.006210566 | | | | |
| CTAG_R2_60 | 0.003922311 | 0.003334011- | 0.003594283 | 0.003533921- | 0.92 | 0.089763068 | 1 | No |
| | | 0.004484507 | | 0.003699434 | | | | |
| TGAT_R2_60 | 0.002106346 | 0.001732068- | 0.001876119 | 0.001625445- | 0.89 | 0.089763068 | 1 | No |
| | | 0.002404058 | | 0.002080450 | | | | |
| CCGC_R2_60 | 0.001626465 | 0.001178868- | 0.001319193 | 0.001215979- | 0.81 | 0.090987794 | 1 | No |
| | | 0.002015147 | | 0.001436147 | | | | |
| AACT_R2_60 | 0.002000407 | 0.001557885- | 0.002197113 | 0.001983752- | 1.10 | 0.093477546 | 1 | No |
| | | 0.002484949 | | 0.002389136 | | | | |
| CTTT_R2_60 | 0.006407733 | 0.004590668- | 0.007098824 | 0.006736049- | 1.11 | 0.097314341 | 1 | No |
| | | 0.008053964 | | 0.007566744 | | | | |
| GTGC_R2_60 | 0.004151005 | 0.002235542- | 0.002841383 | 0.002596870- | 0.68 | 0.097314341 | 1 | No |
| | | 0.005394700 | | 0.003071911 | | | | |
| ACTT_R2_60 | 0.001995444 | 0.001448314- | 0.002182190 | 0.001988975- | 1.09 | 0.111019708 | 1 | No |
| | | 0.002444414 | | 0.002389500 | | | | |
| CGAT_R2_60 | 0.000208128 | 0.000140179- | 0.000223481 | 0.000176222- | 1.07 | 0.119947698 | 1 | No |
| | | 0.000263260 | | 0.000254459 | | | | |
| CCGG_R2_60 | 0.001015256 | 0.000877615- | 0.001056901 | 0.000957818- | 1.04 | 0.190089245 | 1 | No |
| | | 0.001131642 | | 0.001149185 | | | | |
| GGAC_R2_60 | 0.002724951 | 0.001484137- | 0.002012744 | 0.001913908- | 0.74 | 0.21043686 | 1 | No |
| | | 0.003605699 | | 0.002107861 | | | | |
| TGTC_R2_60 | 0.003489090 | 0.003006070- | 0.003225721 | 0.003016486- | 0.92 | 0.212791491 | 1 | No |
| | | 0.003778698 | | 0.003417783 | | | | |
| ACTG_R2_60 | 0.002879708 | 0.002605774- | 0.002933948 | 0.002767415- | 1.02 | 0.217557704 | 1 | No |
| | | 0.003042140 | | 0.003071306 | | | | |
| GTCA_R2_60 | 0.005063752 | 0.003783429- | 0.004324533 | 0.004074065- | 0.85 | 0.269475813 | 1 | No |
| | | 0.006332568 | | 0.004566775 | | | | |
| CTCC_R2_60 | 0.023270857 | 0.019266007- | 0.023149367 | 0.021446375- | 0.99 | 0.280804025 | 1 | No |
| | | 0.025644533 | | 0.025443778 | | | | |
| TTCA_R2_60 | 0.009361174 | 0.008681667- | 0.009134462 | 0.008731558- | 0.98 | 0.286587705 | 1 | No |
| | | 0.010046235 | | 0.009622270 | | | | |
| TTTC_R2_00 | 0.006716593 | 0.005199708- | 0.007235701 | 0.006827222- | 1.08 | 0.362240762 | 1 | No |
| | | 0.008364686 | | 0.007776298 | | | | |
| CTTC_R2_60 | 0.010584038 | 0.008226404- | 0.011264709 | 0.010955610- | 1.06 | 0.46489241 | 1 | No |
| | | 0.013103941 | | 0.011818841 | | | | |
| GGAA_R2_60 | 0.004160979 | 0.003695615- | 0.004275427 | 0.003945788- | 1.03 | 0.468835116 | 1 | No |
| | | 0.004628321 | | 0.004585105 | | | | |
| CCCC_R2_60 | 0.011973092 | 0.009414497- | 0.010918920 | 0.010129620- | 0.91 | 0.472796255 | 1 | No |
| | | 0.013410286 | | 0.011644623 | | | | |
| ATAA_R2_60 | 0.003090622 | 0.002753806- | 0.003152502 | 0.002806855- | 1.02 | 0.5386112 | 1 | No |
| | | 0.003451065 | | 0.003469004 | | | | |
| ATGG_R2_60 | 0.003188199 | 0.002800857- | 0.003065571 | 0.002721532- | 0.96 | 0.581958972 | 1 | No |
| | | 0.003373882 | | 0.003417828 | | | | |
| ACTC_R2_60 | 0.002156082 | 0.001817292- | 0.002132968 | 0.001991086- | 0.99 | 0.645242771 | 1 | No |
| | | 0.002402284 | | 0.002249700 | | | | |
| GGAG_R2_60 | 0.003333455 | 0.002677555- | 0.003400007 | 0.003203205- | 1.02 | 0.659166438 | 1 | No |
| | | 0.003871155 | | 0.003608922 | | | | |
| TeloRV | 0.000397230 | 0.000149972- | 0.000314672 | 0.000215295- | 0.79 | 0.692110895 | 1 | Yes |
| | | 0.000521811 | | 0.000334726 | | | | |
| TGTT_R2_60 | 0.002825088 | 0.002399292- | 0.002799202 | 0.002484838- | 0.99 | 0.735331544 | 1 | No |
| | | 0.003162081 | | 0.003152011 | | | | |
| ATCA_R2_60 | 0.004863209 | 0.004452241- | 0.004841121 | 0.004550124- | 1.00 | 0.819194768 | 1 | No |
| | | 0.005143113 | | 0.005223502 | | | | |
| ATGC_R2_60 | 0.002788962 | 0.002282426- | 0.002721835 | 0.002458605- | 0.98 | 0.819194768 | 1 | No |
| | | 0.003212905 | | 0.003019946 | | | | |
| CCGA_R2_60 | 0.000821187 | 0.000680002- | 0.000797247 | 0.000709357- | 0.97 | 0.824203437 | 1 | No |
| | | 0.000925186 | | 0.000882200 | | | | |
| GGCG_R2_60 | 0.000623694 | 0.000526133- | 0.000629491 | 0.000557283- | 1.01 | 0.844307819 | 1 | No |
| | | 0.000732159 | | 0.000694058 | | | | |
| TACT_R2_60 | 0.003324063 | 0.002478292- | 0.003155795 | 0.002887017- | 0.95 | 0.894986389 | 1 | No |
| | | 0.004156095 | | 0.003303534 | | | | |
| TTTT_R2_60 | 0.005664690 | 0.004142937- | 0.005649752 | 0.005133385- | 1.00 | 0.905180635 | 1 | No |
| | | 0.007095613 | | 0.006091718 | | | | |
| TTGC_R2_60 | 0.005601004 | 0.004573070- | 0.005347076 | 0.005019139- | 0.95 | 0.910283732 | 1 | No |
| | | 0.006324947 | | 0.005756676 | | | | |
| GTCC_R2_60 | 0.005598283 | 0.003708235- | 0.005153927 | 0.004685834- | 0.92 | 0.915390532 | 1 | No |
| | | 0.006842281 | | 0.005632996 | | | | |
| CGCA_R2_60 | 0.000682229 | 0.000525073- | 0.000667185 | 0.000629124- | 0.98 | 1 | 1 | No |
| | | 0.000826402 | | 0.000702422 | | | | |
| GGTG_R2_60 | 0.004401667 | 0.002940714- | 0.003862642 | 0.003515882- | 0.88 | 1 | 1 | No |
| | | 0.005188754 | | 0.004148811 | | | | |

### Example 12: Telephone on Early Detection of HCC in an Independent HBV Infection Population Cohort

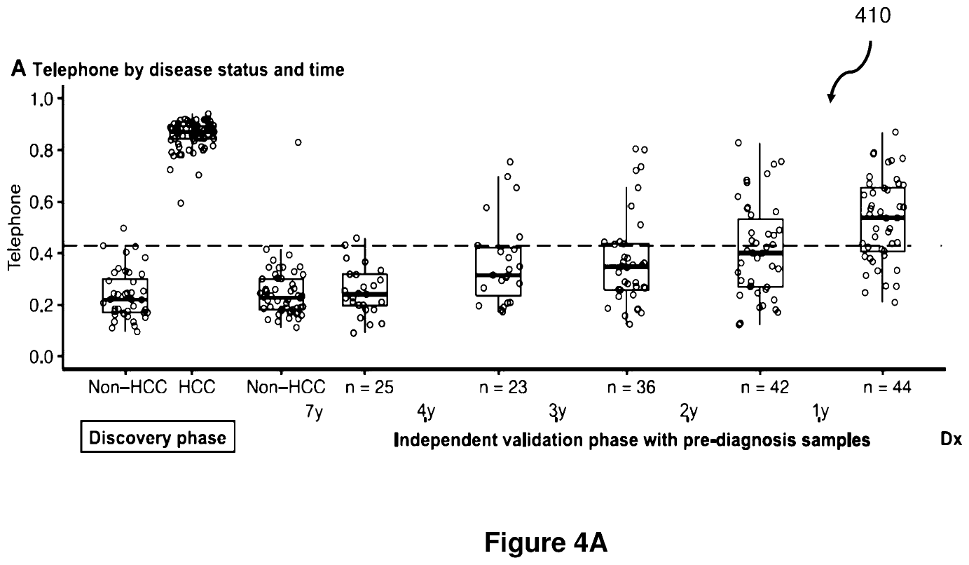

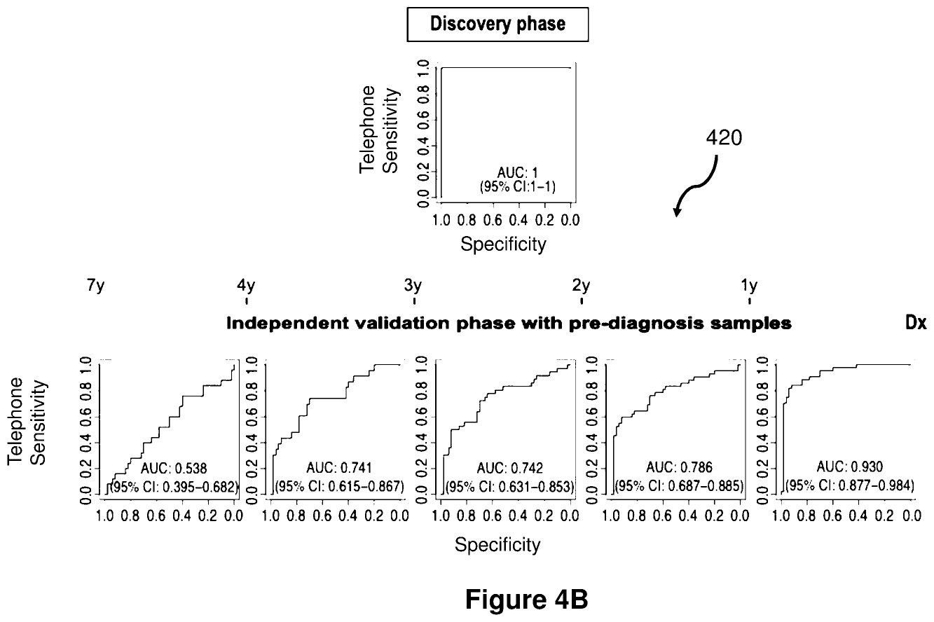

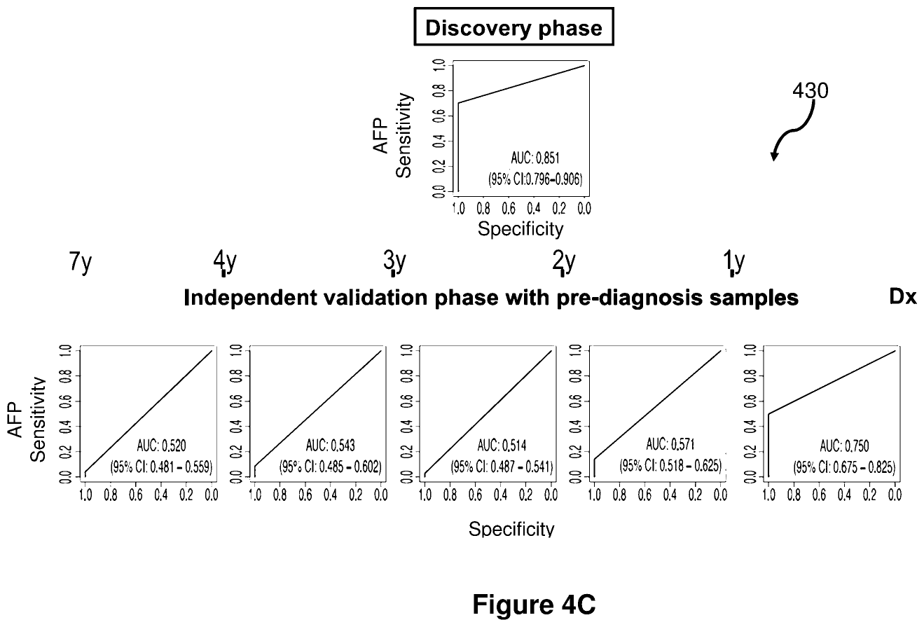

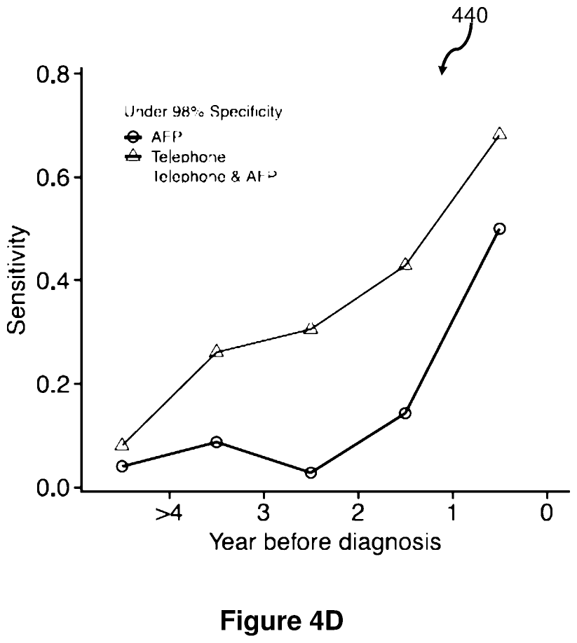

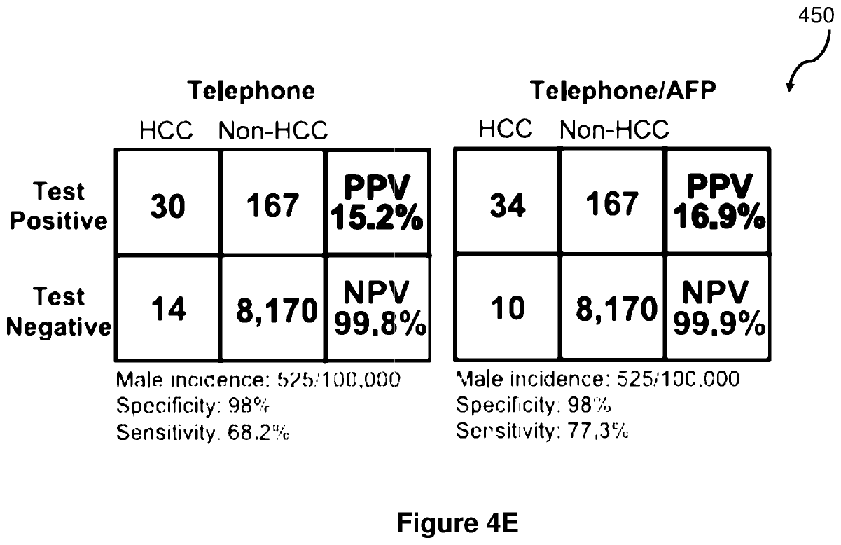

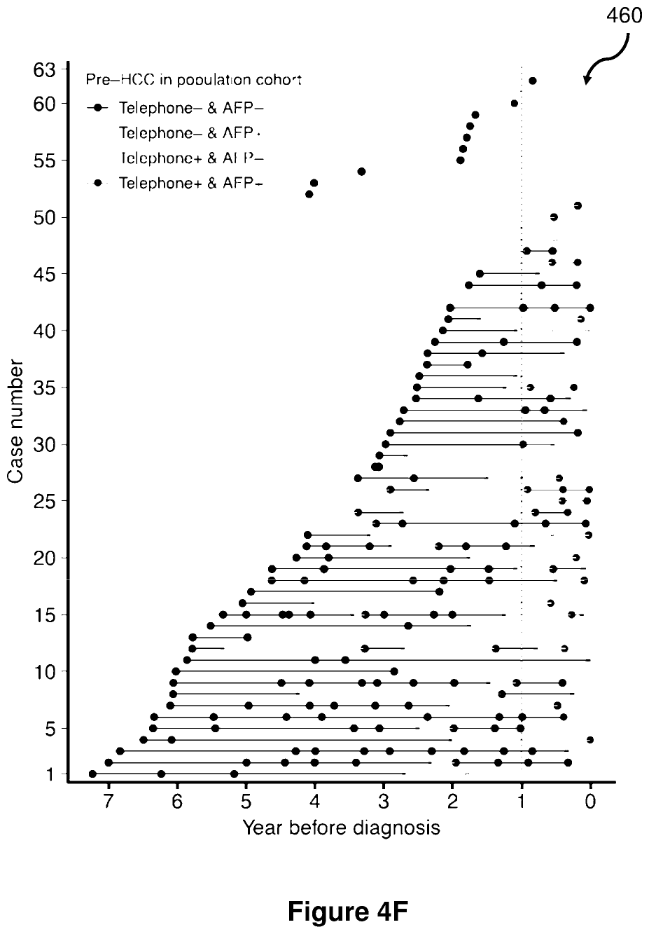

Referring now to FIGS. 4A and 4B. Graph 410 of FIG. 4A shows comparison of Telephone between controls in discovery phase and independent validation phase with pre-diagnosis samples by cutoff curve analysis, and graph 420 of FIG. 4B shows comparison of AUC of Telephone between controls in discovery phase and independent validation phase with pre-diagnosis samples by ROC curve analysis. In the Discovery phase, Telephone completely distinguished controls from HCC cases, with the Telephone mean (±SD) of 0.238 (±0.097) in controls and 0.857 (±0.058) in HCC patients and a corresponding AUC of 1.0. To externally validate the performance of Telephone in early detection of HCC in an independent group of individuals, the Telephone cutoff (0.429) for a specificity of 98% was first determined in the Discovery phase. Next, the fixed model was used to calculate Telephone in an independent Validation cohort comprised of 63 HCC cases (with 270 repeated pre-HCC samples) and 50 controls nested within the population-based liver cancer screening trial. Among the Validation cohort, Telephone increased overtime in the pre-HCC blood samples collected at the intervals of 4 or more years, 3-4 years, 2-3 years, 1-2 years, and within 1 year before diagnosis with means of 0.252, 0.365, 0.373, 0.411, and 0.527, respectively, and was 0.249 among controls (FIG. 4A). Correspondingly, the discriminatory power AUC (95% CI) of Telephone were 0.538 (0.395-0.682), 0.741 (0.615-867), 0.742 (95% CI=0.631-0.853), 0.786 (95% CI=0.687-0.885), and 0.930 (0.877-0.984), respectively (FIG. 4B).

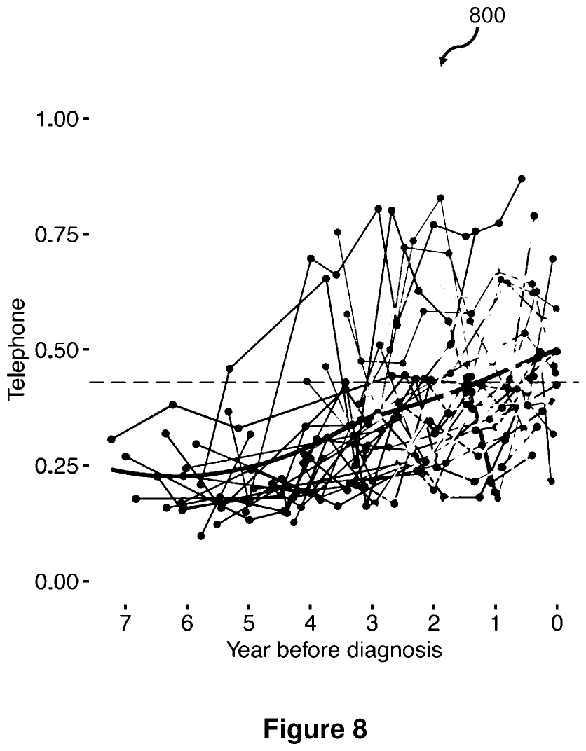

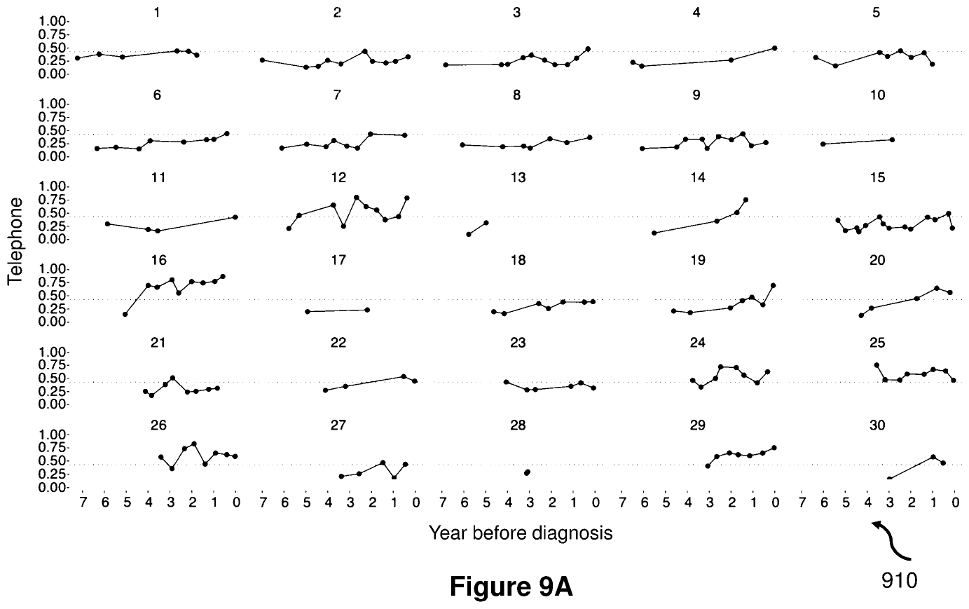

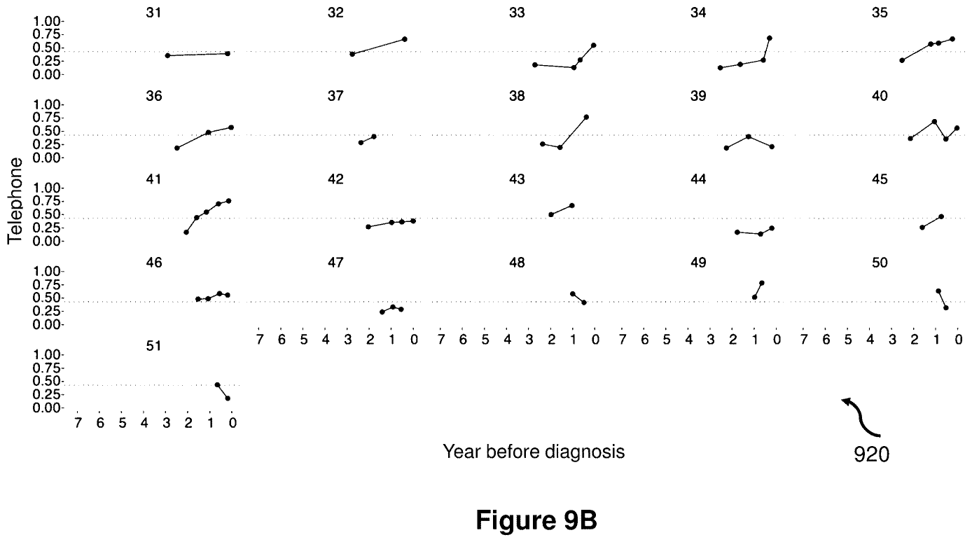

As AFP is widely used as a tumor marker to diagnose HCC, diagnostic performances between AFP and Telephone were also compared. Table 6 shows sensitivity under 98% and 90% specificity of Telephone alone, Telephone & AFP and/or AFP alone with corresponding 95% confidence interval. Referring now to FIG. 4C. Graph 430 of FIG. 4C shows comparison of AUC of AFP between controls in discovery phase and independent validation phase with pre-diagnosis samples by ROC curve analysis. When using AFP alone (>20 ng/mL considered positive), the AUCs (95% CI) were 0.520 (0.481-0.559), 0.543 (0.485-0.602), 0.514 (0.487-0.541), 0.571 (0.518-0.625) and 0.750 (0.675-0.825) for the corresponding intervals before diagnosis, respectively (FIG. 4C). Referring now to FIG. 4D. Graph 440 of FIG. 4D shows the comparison of sensitivities for detecting HCC using AFP alone, Telephone alone and both (AFP and Telephone). The sensitivities (95% CI) for detecting HCC using AFP were 4.0% 0.1%-20.4%), 8.7% (1.1%-18.0%), 2.8% (0.1%-14.5%), 14.3% (5.4%-28.5%), and 50.0% (34.6/0-65.4%) for the five pre-HCC intervals, respectively (FIG. 4D). Compared with AFP, Telephone had higher sensitivities at 8% (1%-26%), 26.1% (10.2%-48.4%), 30.6% (16.3%-48.1%), 42.9% (27.7%-59.0%), and 68.2% (52.4%-81.4%) for the five intervals, respectively. The addition of AFP serum level to Telephone improved the detection sensitivity to 77.3% at 0-1 year before diagnosis (AFP alone 50.0%; Telephone alone 68.2%), and to 54.8% at 1-2 year before diagnosis (AFP alone 14.3%; Telephone alone 42.9%) (FIG. 4D). Using Telephone alone, with the estimated specificity of 98% and sensitivity of 68.2%, in a scenario where the annual incidence for HCC was 525 per 100,000 person-years (corresponding to the HCC incidence rate in men in the screening trial), 30 out of 44 HCC patients would be detected within 1 year before diagnosis, yielding a positive predictive value (PPV) of 15.2% and a negative predictive value (NPV) of 99.8%. Adding AFP would improve the PPV to 16.9% and NPV to 99.9% (FIG. 4D). Now referring to FIGS. 4F, 8 and 9A-9B. Graph 460 of FIG. 4F shows the timeline of pre-HCC blood sample collection in the population cohort. Each line represents one individual. Each dot represents one sampling time point. The statues of Telephone (positive or negative) and AFP (positive or negative) for any blood sample were shown as in the legend. FIGS. 8 and 9A-9B show the dynamic change of Telephone along the time to diagnosis in 51 HCC patients in whom more than two pre-diagnosis samples were available. In FIGS. 8 and 9A-9B, the dotted line is the Telephone at a cutoff (0.429) with a corresponding specificity at 98%. Graph 800 of FIG. 8 shows Telephone changes in a group of pre-HCC patient samples. The solid line shows the Telephone change over time derived by the method of locally estimated scatterplot smoothing. Linear mixed model is used to test the time change trend, and with P<0.001. Graphs 910 and 920 of FIG. 9A-B shows individual Telephone change along the time to diagnosis. Among patients with at least two pre-diagnosis samples, 94% (48/51) had an increased Telephone over time (FIG. 4F and FIGS. 8 and 9A-B), changed from below to above the Telephone cutoff 0.429 in 28 patients (54.9% of 51) later diagnosed with HCC clinically. The median time between the change and clinical HCC diagnosis was 28.1 months (range: 5.0-79.2 months).

TABLE 6
Sensitivity under 98% and 90% specificity of Telephone alone, Telephone
& AFP, and/or AFP alone with corresponding 95% confidence interval.

| | ≥4 year | | 3-4 year | | 2-3 year | |
|---|---|---|---|---|---|---|
| | no./ | | no./ | | no./ | |
| Group | Total no. | Sensitivity (95% CI) | Total no. | Sensitivity (95% CI) | Total no. | Sensitivity (95% CI) |
| 98% specificity | | | | | | |
| Telephone | 2/25 | 8.0% (1.0%-26.0%) | 6/23 | 26.1% (10.2%-48.4%) | 11/36 | 30.6% (16.3%-48.1%) |
| Telophone&AFP | 3/25 | 12.0% (2.5%-31.2%) | 8/23 | 34.8% (16.4%-57.3%) | 12/36 | 33.3% (18.6%-51.0%) |
| 90% specificity | | | | | | |
| Telephone | 2/25 | 8.0% (1.0%-26.0%) | 8/23 | 34.8% (16.4%-57.3%) | 12/36 | 33.3% (18.6%-51.0%) |
| Telophone&AFP | 3/25 | 12.0% (2.5%-31.2%) | 10/23 | 43.5% (23.2%-65.5%) | 13/36 | 36.1% (20.8%-53.8%) |
| AFP | 1/25 | 4.0% (0.1%-20.4%) | 2/23 | 8.7% (1.1%-28.0%) | 1/36 | 2.8% (0.1%-14.5%) |

| | 1-2 year | | 0-1 year | |
|---|---|---|---|---|
| | no./ | | no./ | |
| Group | Total no. | Sensitivity (95% CI) | Total no. | Sensitivity (95% CI) |
| 98% specificity | | | | |
| Telephone | 18/42 | 42.9% (27.7%-59.0%) | 30/44 | 68.2% (52.4%-81.4%) |
| Telophone&AFP | 23/42 | 54.8% (38.7%-70.2%) | 34/44 | 77.3% (62.2%-88.5%) |
| 90% specificity | | | | |
| Telephone | 22/42 | 52.4% (36.4%-68.0%) | 35/44 | 79.5% (64.7%-90.2%) |
| Telophone&AFP | 26/42 | 61.9% (45.6%-76.4%) | 37/44 | 84.1% (69.9%-93.4%) |
| AFP | 6/42 | 14.3% (5.4%-28.5%) | 22/44 | 50.0% (34.6%-65.4%) |

### Example 13: Telephone and Survival in Clinical HCC Patients

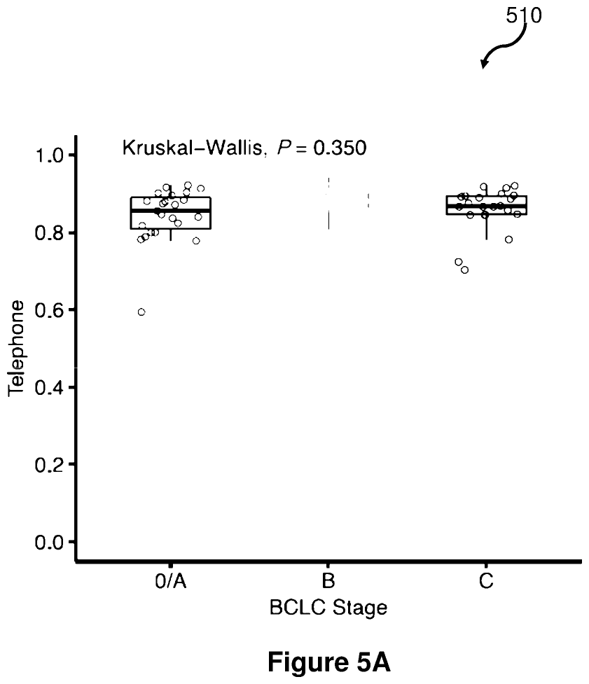

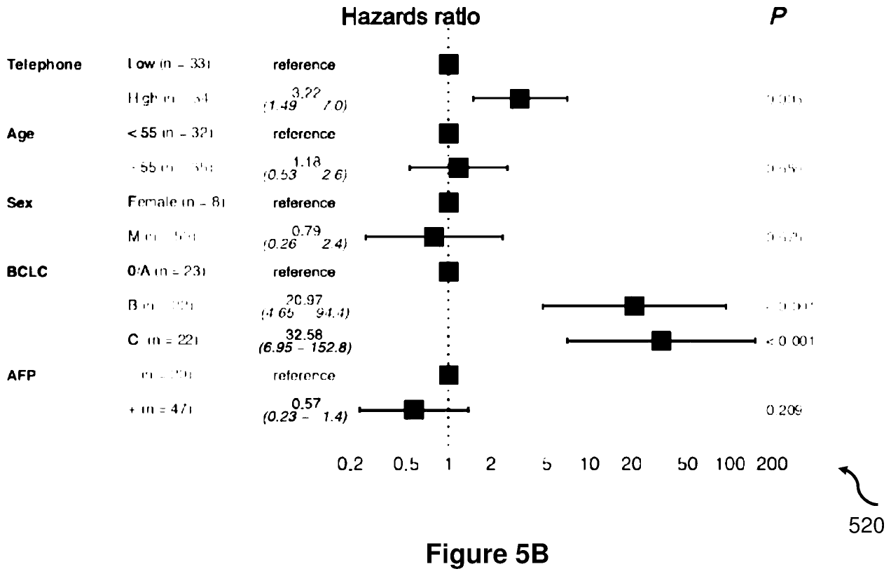

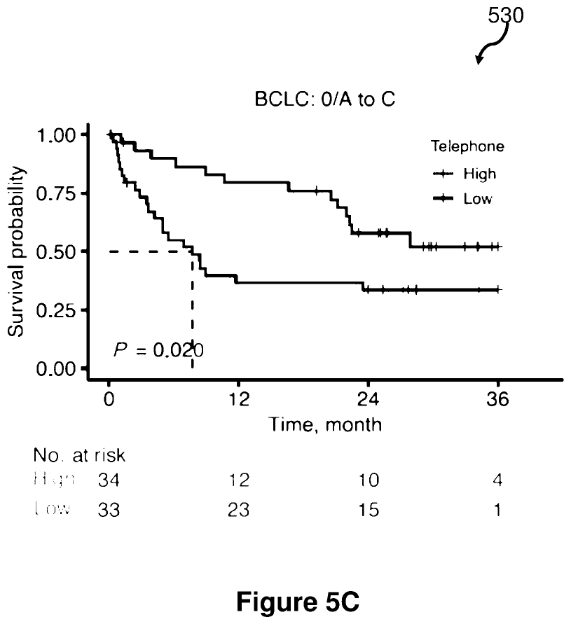

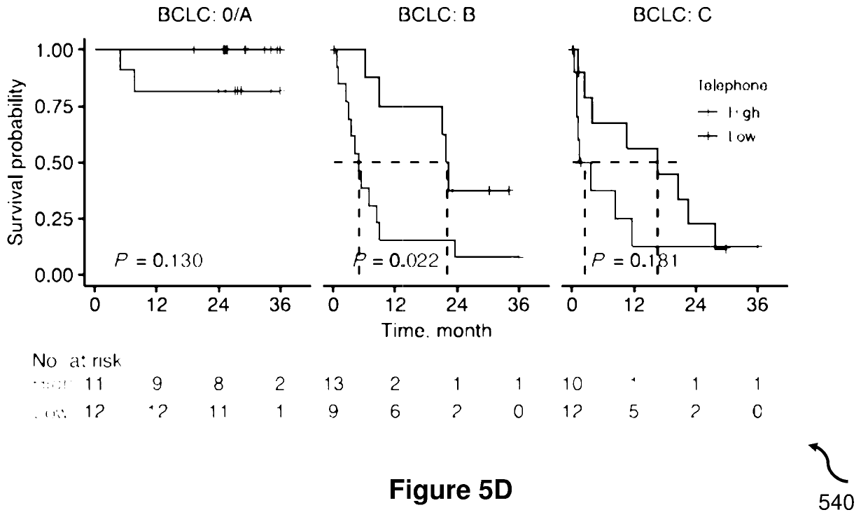

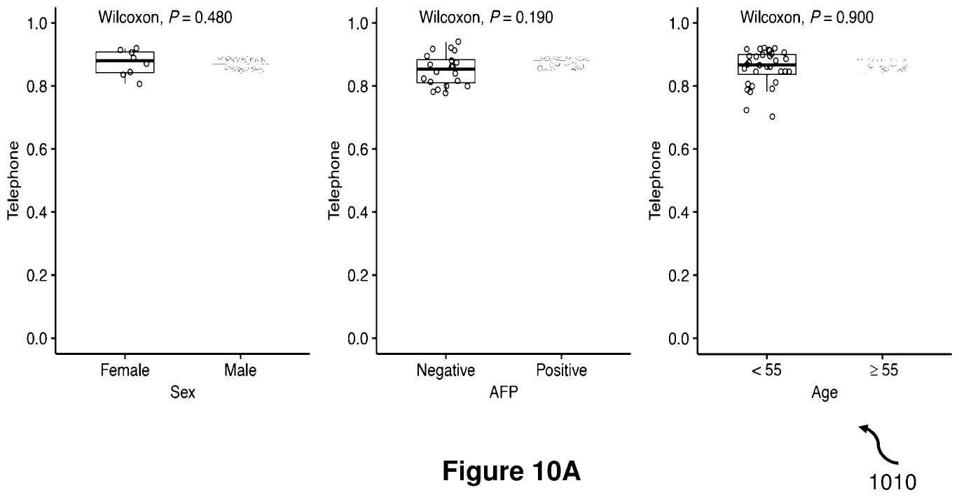

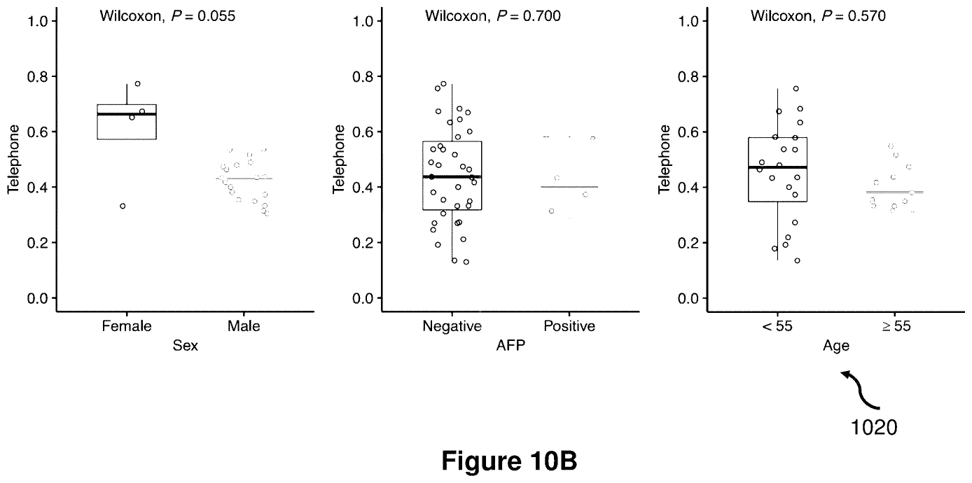

Referring now to FIGS. 5A and 10A-10B. Graph 510 of FIG. 5A shows Kruskal-Wallis tests of Telephone in different BCLC stages. Graph 1010 of FIG. 10A and graph 1020 of FIG. 10B show Telephone distribution by sex, AFP and age in 67 HCC patients in discovery phase (FIG. 10A) and 43 Pre-HCC samples around 1 year before diagnosis in validation phase (FIG. 10B) respectively. Potential clinical factors associated with Telephone were next detected. Differences in Telephone by sex or age (<55 vs ≥55) among cases were not observed, nor among controls, by AFP level (negative vs positive) (FIG. 10A-10B), or by clinical stage when samples were collected at diagnosis (FIG. 5A). It was therefore hypothesized that Telephone may have a prognostic impact on patients' survival that is independent of clinical stage. To test the hypothesis, the association between Telephone score and HCC survival in cases recruited in the Discovery phase was investigated. Among 67 HBV-related HCC cases, 35 deaths (52.2%) were observed after a 36-month follow-up time from diagnosis, with a median survival of 22.2 months. Telephone was categorized into high (>0.868; N=34) and low (≤0.868; N=33) groups. Now referring to FIG. 5B. Graph 520 of FIG. 5B shows hazards ratios of patient survival by factors of Telephone, Age, Sex, BCLC and AFP. After adjustment for sex and age at diagnosis, BLCL stage, and AFP level, HCC patients with high Telephone, compared with low Telephone, had an increased risk of death (hazard ratio 3.22; 95% CI 1.49-7.0, P=0.003) (FIG. 5B). Now referring to FIG. 5C. Graph 530 of FIG. 5C shows the survival probability of HCC patients with high or low Telephone over the time. The survival of HCC patients with high Telephone was shorter than that of low Telephone (median 7.7 months vs not reached; log-rank P=0.020) (FIG. 5C). When stratified by stage, high Telephone was associated with poor survival across all BCLC stages, particularly in Stage B (log-rank P=0.022).

### Example 14: Motif Diversity Score (MDS)

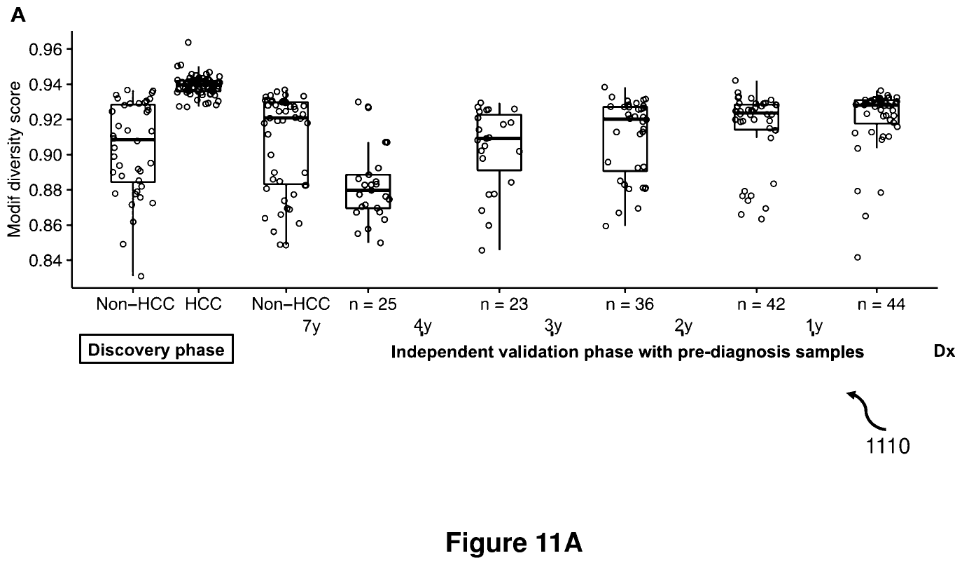

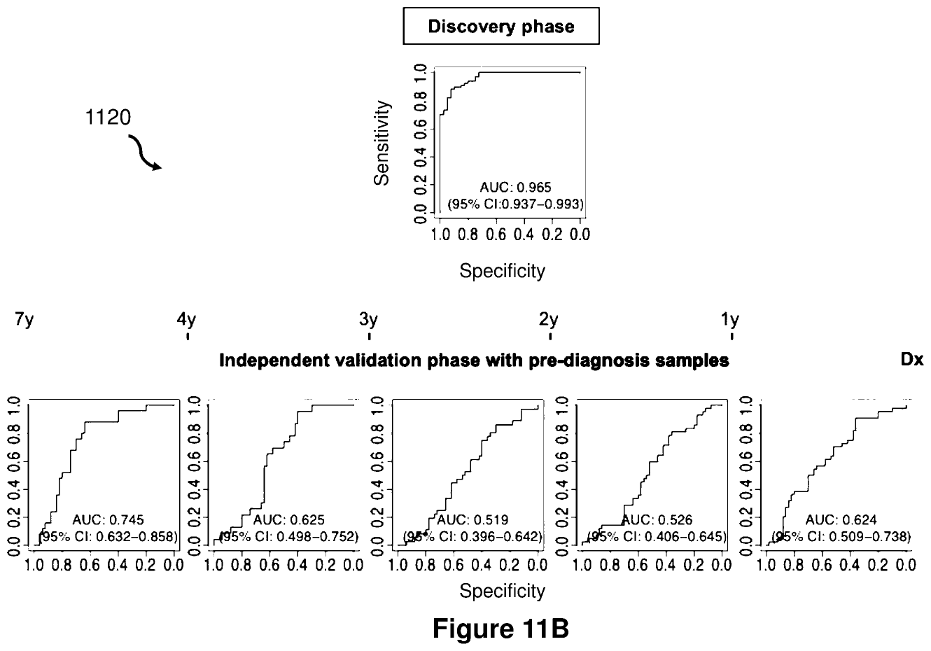

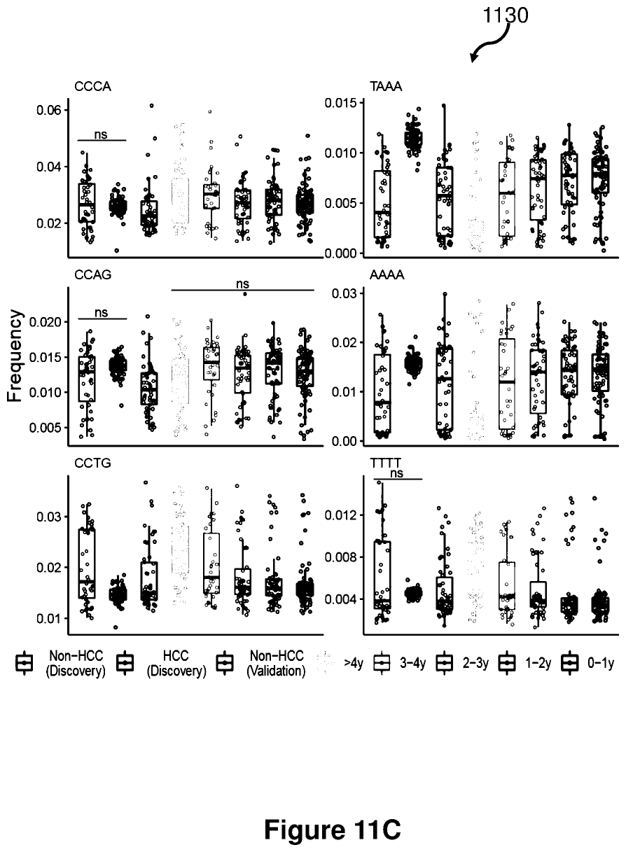

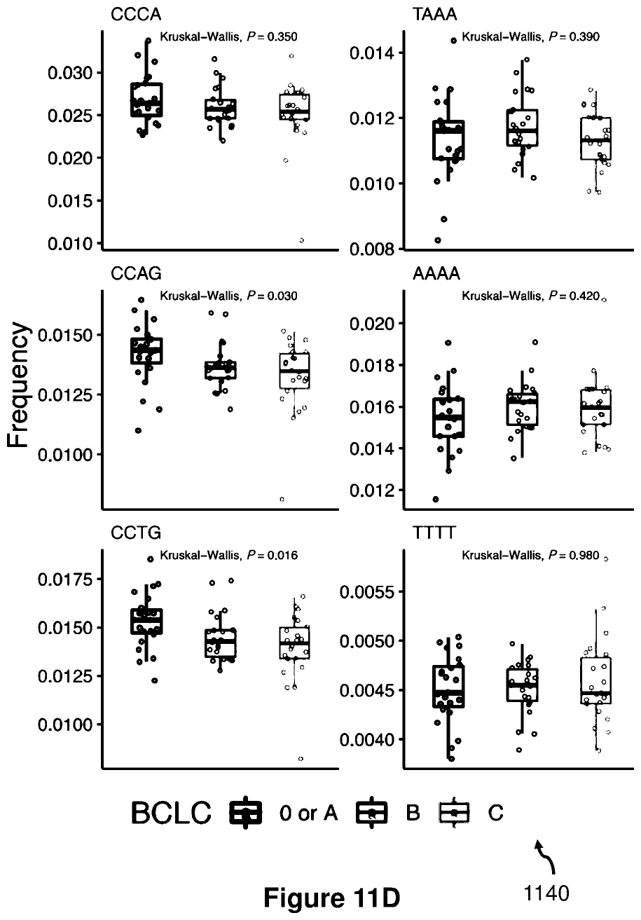

The diversity of fragment end sequence, termed motif diversity score (MDS) previously, in cfDNA was shown to be different in HCC cases from controls. MDS was calculated using 5′ end sequences (the same source as in the Jiang et al.) from fragments longer than 60 nt. Referring now to FIG. 11A. Graph 1110 of FIG. 11A shows motif diversity score (MDS) distribution of Non-HCC and HCC/Pre-HCC in discovery and validation phases. Pre-HCC samples are classified into 5 intervals at >4, 3-4, 2-3, 1-2, and 0-1 year before diagnosis according to the samples collection time. And when more than one sample was evaluated at an interval for one Pre-HCC subject, the mean MDS score is selected. Consistently, MDS in the study was also higher in HCC cases than in controls when blood samples were collected at diagnosis in the Discovery phase (median score 0.940 vs 0.908; P<0.001) (FIG. 11A). The MDS also showed a general increasing trend over time in the five pre-HCC intervals (FIG. 11A). Referring now to FIG. 11B. Graph 1120 of FIG. 11B shows AUC of ccfDNA motif diversity score (MDS) in discovery and validation phases. At diagnosis, the AUC of MDS in distinguishing HCC cases from controls was 0.965 (95% CI 0.937-0.993; FIG. 11B), higher than that reported previously (AUC 0.86)¹³. However, the MDS had limited ability to identify HCC cases when blood samples were collected before clinical diagnosis with the range of AUC only at 0.519-0.745 in the pre-HCC years (FIG. 11B). The distribution of six representative end sequences reported previously (CCCA, CCTG, CCAG, TAAA, AAAA and TTTT) was also investigated. Referring now to FIG. 11C. Graph 1130 of FIG. 11C shows distribution of 6 previous reported end sequence (CCCA, CCAG, CCTG, TAAA, AAAA, TTTT) in discovery and validation phases. Except for non-significant (ns) marked, other groups showed statistically significant difference. Consistent with the study of Jiang et al., TAAA and AAAA showed higher proportions in HCC patients than in controls (both P<0.001) in Discovery phase. However, no differences in the proportions of CCCA, CCTG, CCAG, or TTTT were observed between HCC cases and controls in the study (FIG. 11C). Another study reported an association between tumor burden and these six end sequence these six end sequences by BCLC stage were therefore compared. Referring now to FIG. 11D. Graph 1140 of FIG. 11D shows CCCA, CCAG, CCTG, TAAA, AAAA, TTTT end sequence distribution by BCLC stage in the 67 HCC patients from discovery phase. The result showed high proportions of CCAG (P=0.030) and CCTG (P=0.016) were associated with a late BCLC stage (FIG. 11D).

### Example 15: AUC Values from the 18 Analysis Strata and by the Time Before Diagnosis in the Validation Phase

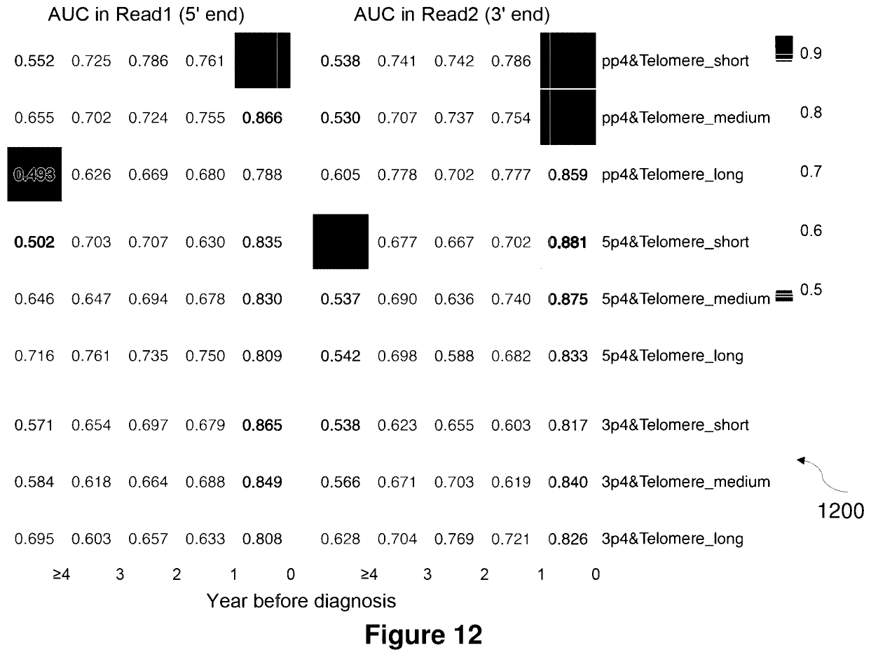

Referring now to FIG. 12, graph 1200 shows all AUC values from the 18 analysis strata and by the time before diagnosis in the Validation phase, following LASSO models developed from respective stratum in the Discovery phase, according to an example embodiment. Patients included in the Discovery phase and Validation phase were mutually exclusive. The 18 strata include stratification by end source (5′/3′), fragment size (short/medium/long), and type of end sequence (5p4/3p4/pp4). Table 7 shows AUC value with corresponding 95% confidence interval in validation phase of 18 LASSO based models developed from 18 strata features respectively.

TABLE 7
AUC value with corresponding 95% confidence interval in validation phase
of 18 LASSO based models developed from 18 strata features respectively.

| | AUC in Read1 (5′ end) | | | | | AUC in Read2 (3′ end) | | | | |
|---|---|---|---|---|---|---|---|---|---|---|
| | ≥4 | 3-4 | 2-3 | 1-2 | 0-1 | ≥4 | 3-4 | 2-3 | 1-2 | 0-1 |
| Group | year | year | year | year | year | year | year | year | year | year |
| pp4& | 0.552 | 0.725 | 0.786 | 0.761 | 0.920 | 0.538 | 0.741 | 0.742 | 0.786 | 0.930 |
| Telo_short | (0.407- | (0.599- | (0.685- | (0.661- | (0.862- | (0.395- | (0.615- | (0.631- | (0.687- | (0.877- |
| | 0.697) | 0.852) | 0.887) | 0.862) | 0.977) | 0.682) | 0.867) | 0.853) | 0.885) | 0.984) |
| pp4& | 0.655 | 0.702 | 0.724 | 0.755 | 0.866 | 0.530 | 0.707 | 0.737 | 0.754 | 0.909 |
| Telo_medium | (0.529- | (0.572- | (0.613- | (0.649- | (0.791- | (0.381- | (0.572- | (0.632- | (0.653- | (0.850 |
| | 0.781) | 0.831) | 0.836) | 0.861) | 0.941) | 0.680) | 0.842) | 0.842) | 0.854) | 0.968) |
| pp4& | 0.493 | 0.626 | 0.669 | 0.680 | 0.788 | 0.605 | 0.778 | 0.702 | 0.777 | 0.859 |
| Telo_long | (0.351- | (0.468- | (0.552- | (0.569- | (0.691- | (0.465- | (0.651- | (0.584- | (0.682- | (0.785- |
| | 0.635) | 0.784) | 0.787) | 0.791) | 0.886) | 0.744) | 0.906) | 0.819) | 0.872) | 0.933) |
| 5p4& | 0.502 | 0.703 | 0.707 | 0.630 | 0.835 | 0.473 | 0.677 | 0.667 | 0.702 | 0.881 |
| Telo_short | (0.356- | (0.563- | (0.588- | (0.507- | (0.749- | (0.331- | (0.534- | (0.547- | (0.592- | (0.811- |
| | 0.647) | 0.844) | 0.826) | 0.753) | 0.922) | 0.615) | 0.819) | 0.787) | 0.813) | 0.952) |
| 5p4& | 0.646 | 0.647 | 0.694 | 0.678 | 0.830 | 0.537 | 0.690 | 0.636 | 0.740 | 0.875 |
| Telo_medium | (0.510- | (0.499- | (0.580- | (0.568- | (0.748- | (0.404- | (0.552- | (0.517- | (0.638- | (0.807- |
| | 0.781) | 0.795) | 0.808) | 0.788) | 0.913) | 0.670) | 0.827) | 0.755) | 0.843) | 0.944) |
| 5p4& | 0.716 | 0.761 | 0.735 | 0.750 | 0.809 | 0.542 | 0.698 | 0.588 | 0.682 | 0.833 |
| Telo_long | (0.598- | (0.634- | (0.625- | (0.649- | (0.722- | (0.406- | (0.551- | (0.463- | (0.571- | (0.749- |
| | 0.834) | 0.888) | 0.845) | 0.850) | 0.896) | 0.679) | 0.846) | 0.713) | 0.793) | 0.918) |
| 3p4& | 0.571 | 0.654 | 0.697 | 0.679 | 0.865 | 0.538 | 0.623 | 0.655 | 0.603 | 0.817 |
| Telo_short | (0.429- | (0.508- | (0.570- | (0.559- | (0.784- | (0.392- | (0.464- | (0.526- | (0.475- | (0.721- |
| | 0.714) | 0.800) | 0.824) | 0.799) | 0.947) | 0.683) | 0.783) | 0.784) | 0.730) | 0.912) |
| 3p4& | 0.584 | 0.618 | 0.664 | 0.688 | 0.849 | 0.566 | 0.671 | 0.703 | 0.619 | 0.840 |
| Telo_medium | (0.450- | (0.478- | (0.546- | (0.576- | (0.769- | (0.417- | (0.518- | (0.585- | (0.498- | (0.758- |
| | 0.718) | 0.759) | 0.783) | 0.800) | 0.929) | 0.714) | 0.824) | 0.821) | 0.739) | 0.921) |
| 3p4& | 0.695 | 0.603 | 0.657 | 0.633 | 0.808 | 0.628 | 0.704 | 0.769 | 0.721 | 0.826 |
| Telo_long | (0.569- | (0.455- | (0.536- | (0.517- | (0.717- | (0.492- | (0.563- | (0.666- | (0.611- | (0.742- |
| | 0.821) | 0.750) | 0.779) | 0.749) | 0.898) | 0.764) | 0.845) | 0.871) | 0.831) | 0.910) |

### Example 16: Library Efficiency Analysis of BLESSING Compared to Conventional Method

Two new BLESSING libraries using an HCC cell line and sequenced to high number of reads were constructed to estimate the efficiency. HepG2 (ATCC, CRL11997) cell lines were purchased from ATCC (American Type Culture Collection, VA, USA) and were cultured in Eagle's Minimum Essential Medium (ATCC, 30-2003) supplemented with 10% fetal bovine serum (GIBCO, 10270-106) and incubated at 37° C. with 5% CO2 in a constant temperature incubator. Two DNA samples were extracted from culture mediums after 72 h of culturing the HepG2 cells. BLESSING libraries were constructed using 30 ng of DNA each and yielded 68M and 82M reads, respectively.

The efficiency of the BLESSING method is compared with the efficiency of a single-stranded library construction method (hereinafter referred to as Snyder's method) as described in Snyder et al. (Snyder M W, Kircher M, Hill A J, Daza R M, Shendure J. Cell-free DNA Comprises an In Vivo Nucleosome Footprint that Informs Its Tissues-Of-Origin. Cell. 2016 Jan. 14; 164(1-2):57-68. doi: 10.1016/j.cell.2015.11.050.).

Briefly, the Snyder's method of preparing single-stranded sequencing libraries is as follows: An adaptor (Adapter 2) was prepared by combining 4.5 ul TE (pH 8), 0.5 ul 1M NaCl, 10 uL 500 uM oligo Adapter2.1 (first strand of Adaptor 2), and 10 ul 500 uM oligo Adapter2.2 (second strand of Adaptor 2), incubating at 95° C. for 10 seconds, and ramping to 14° C. at a rate of 0.1° C./s. Purified cfDNA fragments were dephosphorylated by combining 2× CircLigase II buffer (Epicentre), 5 mM MnCl2, and 1U FastAP (Thermo Fisher) with 0.5-10 ng fragments in 20 ul reaction volume and incubating at 37° C. for 30 minutes. Fragments were then denatured by heating to 95° C. for 3 minutes, and were immediately transferred to an ice bath. The reaction was supplemented with biotin-conjugated adapter oligo CL78 (5 μmol), 20% PEG-6000 (w/v), and 200U CircLigase II (Epicentre) for a total volume of 40 ul, and was incubated overnight with rotation at 60° C., heated to 95° C. for 3 minutes, and placed in an ice bath. For each sample, 20 ul MyOne C1 beads (Life Technologies) were twice washed in bead binding buffer (BBB) (10 mM Tris-HCl [pH 8], 1M NaCl, 1 mM EDTA [pH 8], 0.05% Tween-20, and 0.5% SDS), and resuspended in 250 ul BBB. Adapter-ligated fragments were bound to the beads by rotating for 60 minutes at room temperature. Beads were collected on a magnetic rack and the supernatant was discarded. Beads were washed once with 500 ul wash buffer A (WBA) (10 mM Tris-HCl [pH 8], 1 mM EDTA [pH 8], 0.05% Tween-20, 100 mM NaCl, 0.5% SDS) and once with 500 ul wash buffer B (WBB) (10 mM Tris-HCl [pH 8], 1 mM EDTA [pH 8], 0.05% Tween-20, 100 mM NaCl). Beads were combined with 1× Isothermal amplification Buffer (NEB), 2.5 uM oligo CL9, 250 uM (each) dNTPs, and 24U Bst 2.0 DNA Polymerase (NEB) in a reaction volume of 50 ul, incubated with gentle shaking by ramping temperature from 15° C. to 37° C. at 1° C./minute, and held at 37° C. for 10 minutes. After collection on a magnetic rack, beads were washed once with 200 ul WBA, resuspended in 200 ul of stringency wash buffer (SWB) (0.1×SSC, 0.1% SDS), and incubated at 45° C. for 3 minutes. Beads were again collected and washed once with 200 ul WBB. Beads were then combined with 1× CutSmart Buffer (NEB), 0.025% Tween-20, 100 uM (each) dNTPs, and 5U T4 DNA Polymerase (NEB) and incubated with gentle shaking for 30 minutes at room temperature. Beads were washed once with each of WBA, SWB, and WBB as described above. Beads were then mixed with 1× CutSmart Buffer (NEB), 5% PEG-6000, 0.025% Tween-20, 2 uM double-stranded Adapter 2, and 10 U T4 DNA Ligase (NEB), and incubated with gentle shaking for 2 hours at room temperature. Beads were washed once with each of WBA, SWB, and WBB as described above, and resuspended in 25 ul TET buffer (10 mM Tris-HCl [pH 8], 1 mM EDTA [pH 8], 0.05% Tween-20). Second strands were eluted from beads by heating to 95° C., collecting beads on a magnetic rack, and transferring the supernatant to a new tube. Library amplification was monitored by real-time PCR, requiring an average of 4-6 cycles per library.

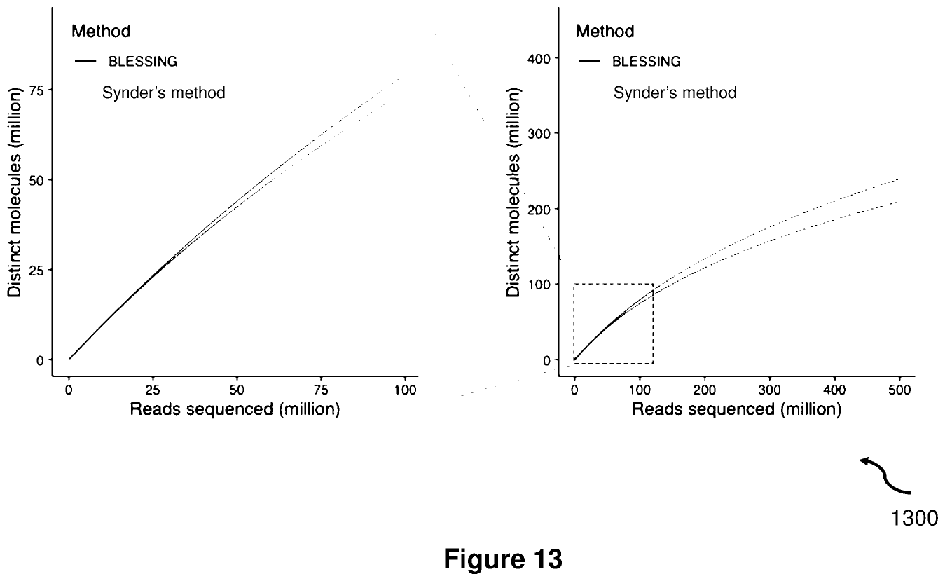

Referring now to FIG. 13, graph 1300 shows a comparison of library complexity of BLESSING with the Snyder's method. As shown in FIG. 13, BLESSING method had comparable efficiency in terms of conversion of DNA fragments to sequence-able library with Snyder's method.

### Example 17: Library Efficiency Analysis

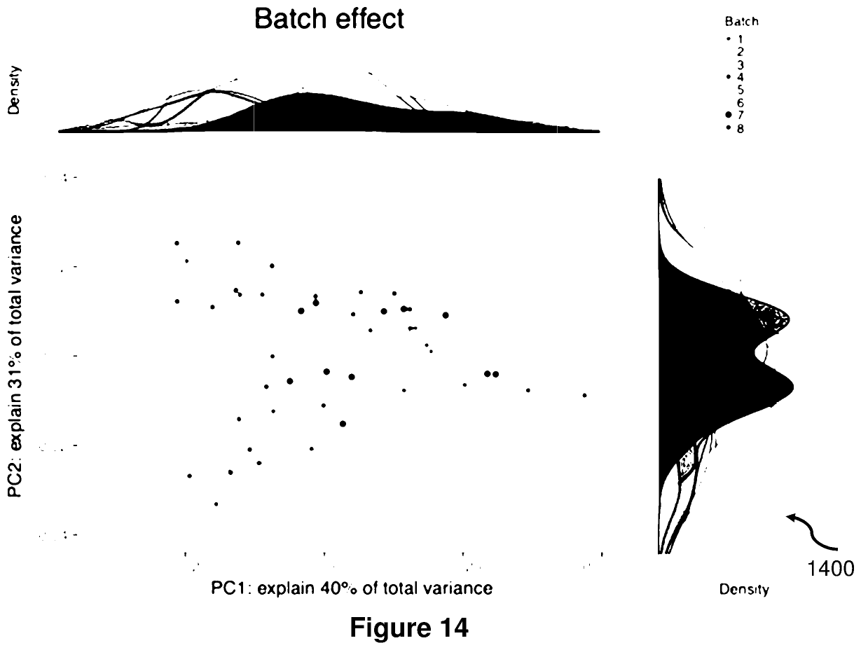

Now referring to FIG. 14. Graph 1400 of FIG. 14 shows the principle component analysis of non-HCC controls by experiment batch. The principle component analysis (PCA) approach was adapted to evaluate the potential batch effect. The total 90 non-HCC controls constructed in eight batches of sequencing libraries were used in the analysis. No significant batch effect was observed based on the principle component analysis approach using all 260 fragmentation features (FIG. 14).

### Example 18: Evaluation of Telecon Model Using Data from Snyder et al.

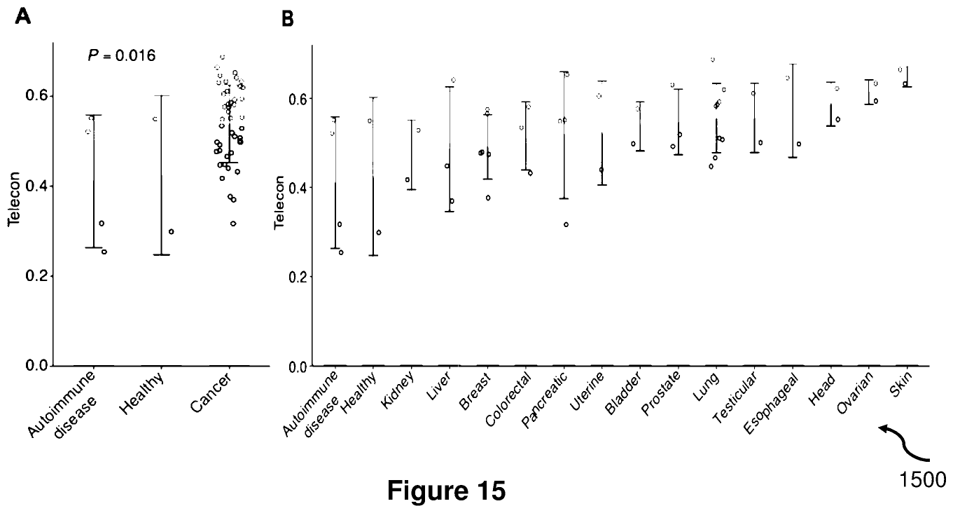

Telecon model was evaluated using data from Snyder et al., 2016 (Table S1, S4 and Table S5 of the Supplementary data from Snyder et al.), which also contained single-strand sequencing data. Referring now to FIG. 15, graph 1500 shows the external evaluation of Telecon score with multiple cancers using data from Snyder et al. The results showed that Telecon scores differed significantly among healthy, autoimmune disease, and cancer group that consisted of 14 different tissue types, including kidney cancer, liver cancer, breast cancer, colorectal cancer, pancreatic cancer, uterine cancer, bladder cancer, prostate cancer, lung cancer, testicular cancer, esophageal cancer, head cancer, ovarian cancer, and skin cancer (P=0.016, FIG. 15). This provides a further and independent supporting evidence for our methodology and for using circulating telomere DNA as a promising biomarker for early detection of cancer.

### Example 19: A Method of Predicting or Detecting Cancer in a Subject by Performing Quantitative Analysis of Telomere-Containing Sequences

In one embodiment, provided is a method of predicting or detecting cancer in a human subject, including the steps of: (a) obtaining a sample including a plurality of nucleic acid fragments from the subject; and (b) performing a quantitative analysis of the level of at least one biomarker associated with the cancer using the plurality of nucleic acid fragments of the sample, wherein the at least one biomarker includes one or more telomere-containing sequences including at least two consecutive repeats of nucleotide sequence TTAGGG. In some embodiments, the at least one biomarker comprises two consecutive repeats of nucleotide sequence (e.g. TTAGGGTTAGGG (SEQ ID NO: 5)). In some embodiments, the one or more telomere-containing sequences do not comprise a single set of nucleotide sequence TTAGGG with no consecutive repeats. In some embodiments, the at least one biomarker is identified by any of the methods as disclosed in examples above.

In some embodiments, the quantitative analysis includes the steps of quantifying the level of the at least one biomarker in the subject, and comparing the level of the at least one biomarker in the subject against the level of the at least one biomarker in a control group without the cancer.

In some embodiments, the quantitative analysis can be performed by any quantitative methods or quantitative assays for target nucleic acid sequences (e.g. DNA), such as quantitative real-time PCR (qPCR), digital PCR (dPCR), the Amplification Refractory Mutation System PCR (ARMS-PCR), or hybridization-based target enrichments followed by qPCR, ARMS-PCR, mass measurement such as by fluorometry, and molecular counting. In some embodiments, the quantitative analysis is performed by quantitative real-time PCR (qPCR). In some embodiments, the quantitative analysis is performed by quantitative digital PCR (dPCR). In some embodiments, the quantitative PCR is performed by using a target-specific primer pair, wherein at least one primer in the target-specific primer pair is at least partially complementary to the at least one biomarker.

In some embodiments, the cancer is selected from a group consisting of kidney cancer, liver cancer, breast cancer, colorectal cancer, pancreatic cancer, uterine cancer, bladder cancer, prostate cancer, lung cancer, testicular cancer, esophageal cancer, head cancer, ovarian cancer, and skin cancer. In some embodiments, the cancer is hepatocellular carcinoma (HCC).

In some embodiments, the plurality of nucleic acid fragments is prepared by fragmentizing and/or denaturing high molecular weight DNA. In some embodiments, the plurality of nucleic acid fragments includes single-strand cDNA fragments prepared from reverse transcription of RNA fragments.

In some embodiments, the sample is prepared by extracting a blood sample of the subject. In some embodiments, the sample is prepared by isolating cell-free nucleic acids extracted from a blood sample of the subject. In some embodiments, the sample is prepared by isolating nucleic acids extracted from lymphocytes in a blood sample of the subject for T-cell and B-cell receptor profiling. In some embodiments, the sample is prepared by isolating nucleic acids extracted from circulating tumor cells.

### SUMMARY OF RESULTS

Of 18,373 participants, 2,893 were HBV-seropositive and developed 81 incident HCC cases. Among short ccfDNA (25-60 nucleotides), telomere G-tail was more abundant in HCC patients than in controls (18.87-fold, P=6.4×10−18). Telomere contributed 91% of the variation of the Telephone model, which distinguished HCC cases from controls completely (AUC=1.0). In Validation, Telephone showed increasing detection performance using pre-HCC samples collected ≥4 years (AUC=0.538), 3-4 years (0.741), 2-3 years (0.742), 1-2 years (0.786), and 0-1 year (0.930) before diagnosis. Within one year before diagnosis and at a specificity of 98%, Telephone had a sensitivity of 68.2% (95% CI=52.4-81.4%) in detecting early HCC, yielding an estimated positive predict value of 15.2% among HBV-seropositive population. High Telephone was also associated with poor survival in hospital HCC patients (hazard ratio 3.22, 95% CI=1.49-7.0), independent of tumor stage. Therefore, circulating short telomere G-tail may effectively detect early hepatocellular carcinoma in high-risk populations.

The exemplary embodiments of the present invention are thus fully described. Although the description referred to particular embodiments, it will be clear to one skilled in the art that the present invention may be practiced with variation of these specific details. The methods/steps discussed in one figure can be added to or exchanged with methods/steps in other figures. Hence this invention should not be construed as limited to the embodiments set forth herein.

1. A method of preparing at least one ligation product from a sample comprising a plurality of single-strand nucleic acid fragments, the method comprising the steps of:
(a) ligating a first universal oligonucleotide adaptor to at least one single-strand nucleic acid fragment, wherein the first universal oligonucleotide adaptor is configured for ligating to a 3′ end of individual single-strand nucleic acid fragment; and
(b) ligating a second universal oligonucleotide adaptor to the at least one single-strand nucleic acid fragment, wherein the second universal oligonucleotide adaptor is configured for ligating to a 5′ end of individual single-strand nucleic acid fragment,
thereby at least one ligation product is formed.

2. The method of claim 1, wherein prior to the step (a), the method further comprises the step of:
dephosphorylating the 5′ end of the at least one single-strand nucleic acid fragment;
and/or prior to the step (b), the method further comprises the step of:
phosphorylating the 5′ end of the at least one single-strand nucleic acid fragment.

3. (canceled)

4. The method of claim 1, wherein the first universal oligonucleotide adaptor further comprises:
a top strand having a 5′ recessive end, wherein the 5′ recessive end is configured for ligating to the 3′ end of the individual single-strand nucleic acid fragment; and
a bottom strand partially complementary to the top strand to form a duplex portion,
wherein the duplex portion of the first universal oligonucleotide adaptor is of sufficient length to remain in duplex form in the step (a), wherein the bottom strand of the first universal oligonucleotide adaptor comprises an unpaired 3′ portion;
wherein the second universal oligonucleotide adaptor further comprises:
a to strand having a 3′ recessive end, wherein the 3′ recessive end is configured for ligating to the 5′ end of the individual single-strand nucleic acid fragment; and
a bottom strand partially complementary to the too strand to form a duplex portion,
wherein the duplex portion of the second universal oligonucleotide adaptor is of sufficient length to remain in duplex form in the step (b), wherein the bottom strand of the second universal oligonucleotide adaptor comprises an unpaired 5′ portion.

5-7. (canceled)

8. The method of claim 1, wherein the first universal oligonucleotide adaptor and/or the second universal oligonucleotide adaptor comprise a hairpin loop connecting a portion of the duplex form.

9. The method of claim 1, wherein the first universal oligonucleotide adaptor and/or the second universal oligonucleotide adaptor comprise a single strand segment comprising three to twenty random nucleotides as a unique molecular index (UMI) for tracing individual original molecules.

10. The method of claim 4, wherein the bottom strand of the first universal oligonucleotide adaptor comprises a nucleotide sequence of SEQ ID NO:3, and the top strand of the first universal oligonucleotide adaptor comprises a nucleotide sequence of SEQ ID NO:4; wherein the bottom strand of the second universal oligonucleotide adaptor comprises nucleotide sequence SEQ ID NO:1, and the too strand of the second universal oligonucleotide adaptor comprises a nucleotide sequence of SEQ ID NO:2.

11. (canceled)

12. The method of claim 1, wherein the method further comprises the step of:
amplifying the at least one ligation product with a pair of sequencing specific adaptor primers to form a sequencing library, wherein the pair of sequencing specific adaptor primers is at least partially complementary to the first universal oligonucleotide adaptor and the second universal oligonucleotide adaptor, respectively.

13. The method of claim 12, wherein the method further comprises the step of sequencing the sequencing library using a sequencing primer pair.

14. The method of claim 1, wherein the method further comprises the step of:
enriching at least one targeted nucleic acid from the at least one ligation product, using at least one target specific primer and at least one universal oligonucleotide adaptor primer that is at least partially complementary to the first or second universal oligonucleotide adaptor.

15. The method of claim 1, wherein the sample comprises a plurality of DNA fragments prepared from high molecular weight DNA.

16. The method of claim 1, wherein the plurality of single-strand nucleic acid fragments is prepared from denaturation of double-strand DNA fragments.

17. The method of claim 1, wherein the sample comprises single-strand cDNA fragments prepared from reverse transcription of RNA fragments.

18-19. (canceled)

20. The method of claim 1, wherein the sample is cell-free nucleic acids extracted from a blood sample;
wherein the sample is nucleic acids extracted from lymphocytes in a blood sample for T-cell and B-cell receptor profiling; or
wherein the sample is nucleic acids extracted from circulating tumor cells.

21-43. (canceled)

44. A method of identifying one or more biomarkers associated with a disease or condition, comprising the steps of:
a. obtaining a plurality of samples comprising a plurality of single-strand nucleic acid fragments from a case group of subjects having the disease or condition and from a control group;
b. for individual sample, ligating a first universal oligonucleotide adaptor to at least one single-strand nucleic acid fragment, wherein the first universal oligonucleotide adaptor is configured for ligating to a 3′ end of individual single-strand nucleic acid fragment;
c. ligating a second universal oligonucleotide adaptor to the at least one single-strand nucleic acid fragment, wherein the second universal oligonucleotide adaptor is configured for ligating to a 5′ end of individual single-strand nucleic acid fragment, thereby at least one ligation product is formed;
d. amplifying the at least one ligation product with a pair of sequencing specific adaptor primers to form individual sequencing library, wherein the pair of sequencing specific adaptor primers is at least partially complementary to the first universal oligonucleotide adaptor and the second universal oligonucleotide adaptor respectively,
e. quantifying and reading the sequencing library to obtain individual sequencing result; and
f. comparing the sequencing results between the case group and the control group, such that one or more biomarkers associated with the disease or condition are identified.

45. The method of claim 44, wherein the step (f) comprises the step of:
(i) comparing proportions of individual biomarker between the case group and the control group using Wilcoxon rank-sum test;
(ii) identifying individual biomarker with fold-difference of the proportions that is greater or equal to 2, or lesser or equal to 0.5.

46. The method of claim 44, wherein the step (f) comprises one or moe of the steps of:
(i) evaluating individual identified biomarker using logistic regression model with a Least Absolute Shrinkage and Selection Operator (LASSO) penalty to obtain a LASSO coefficient;
(ii) selecting one or more biomarkers with a non-zero LASSO coefficient among the identified biomarkers;
(iii) formulating a logistic regression model using the LASSO coefficient based on the selected one or more biomarkers, such that a Telomere and end sequence phenomenon etymology (Telephone score is obtained; and
(iv) validating the logistic regression model in a prospective cohort of subjects to determine the performance of the logistic regression model in detecting the disease or condition.

47-48. (canceled)

49. The method of claim 44, wherein the subjects are human.

50. The method of claim 44, wherein the disease or condition is cancer or autoimmune disease, wherein the cancer is selected from a group consisting of kidney cancer, liver cancer, breast cancer, colorectal cancer, pancreatic cancer, uterine cancer, bladder cancer, prostate cancer, lung cancer, testicular cancer, esophageal cancer, head cancer, ovarian cancer, and skin cancer.

51. (canceled)

52. The method of claim 50, wherein the cancer is hepatocellular carcinoma (HCC).

53. The method of claim 44, wherein the one or more biomarkers comprise one or more telomere-related sequences and/or one or more fragment end sequences;
wherein the one or more telomere-related sequences comprise:
(i) one or more telomere-containing sequences comprising at least two consecutive repeats of nucleotide sequence TTAGGG; and
(ii) one or more non-telomere containing sequences that do not comprise nucleotide sequence TTAGGG; and
wherein the one or more fragment end sequences comprise nucleotide sequences CAAA and/or GATG.

54-55. (canceled)

56. The method of claim 44, wherein prior to the step (b), the method further comprises the step of:
dephosphorylating the 5′ end of the at least one single-strand nucleic acid fragment;
and/or prior to the step (c), the method further comprises the step of:
phosphorylating the 5′ end of the at least one single-strand nucleic acid fragment.

57. (canceled)

58. The method of claim 44, wherein the first universal oligonucleotide adaptor further comprises:
a top strand having a 5′ recessive end, wherein the 5′ recessive end is configured far ligating to the 3′ end of the individual single-strand nucleic acid fragment; and
a bottom strand partially complementary to the top strand to form a duplex portion,
wherein the duplex portion of the first universal oligonucleotide adaptor is of sufficient length to remain in duplex form in the step (b), wherein the bottom strand of the first universal oligonucleotide adaptor comprises an unpaired 3′ portion;
wherein the second universal oligonucleotide adaptor further comprises:
a top strand having a 3′ recessive end, wherein the 3′ recessive end is configured for ligating to the 5′ end of the individual single-strand nucleic acid fragment; and
a bottom strand partially complementary to the top strand to form a duplex portion,
wherein the duplex portion of the second universal oligonucleotide adaptor is of sufficient length to remain in duplex form in the step (c), wherein the bottom strand of the second universal oligonucleotide adaptor comprise an unpaired 5′ portion.

59-61. (canceled)

62. The method of claim 44, wherein the first universal oligonucleotide adaptor and/or the second universal oligonucleotide adaptor comprise a hairpin loop connecting a portion of the duplex form.

63. The method of claim 44, wherein the first universal oligonucleotide adaptor and/or the second universal oligonucleotide adaptor comprise three to twenty random nucleotides as a unique molecular index (UMI) for tracing individual original molecules.

64. The method of claim 58, wherein the bottom strand of the first universal oligonucleotide adaptor comprises a nucleotide sequence of SEQ ID NO:3, and the top strand of the first universal oligonucleotide adaptor comprises a nucleotide sequence of SEQ ID NO:4;
wherein the bottom strand of the second universal oligonucleotide adaptor comprises nucleotide sequence SEQ ID NO:1, and the too strand of the second universal oligonucleotide adaptor comprises a nucleotide sequence of SEQ ID NO:2.

65. (canceled)

66. The method of claim 44, wherein the sample comprises a plurality of DNA fragments prepared from high molecular weight DNA.

67. The method of claim 44, wherein the plurality of single-strand nucleic acid fragments is prepared from denaturation of double-strand DNA fragments.

68. The method of claim 44, wherein the sample comprises single-strand cDNA fragments prepared from reverse transcription of RNA fragments.

69. (canceled)

70. The method of claim 44, wherein the sample is cell-free nucleic acids extracted from a blood sample;
wherein the sample is nucleic acids extracted from lymphocytes in a blood sample for T-cell and B-cell receptor profiling; or
wherein the sample is nucleic acids extracted from circulating tumor cells.

71-113. (canceled)


SEQUENCE LISTING (13 sequences)

SEQ ID NO: 1 - DNA, 48 bp, synthetic construct
  source 1..48: mol_type = other DNA
  misc_difference 1..6: note = A or T or C or G
  misc_difference 15..16: note = A or T or C or G
  misc_difference 17: note = A or T
  misc_difference 18..20: note = A or T or C or G
  misc_difference 21: note = A or T
  misc_difference 22..24: note = A or T or C or G
nnnnnngtgtcactnnwnnnwnnnagatcggaagagcgtcgtgtagtt

SEQ ID NO: 2 - DNA, 42 bp, synthetic construct
  source 1..42: mol_type = other DNA
  modified_base 1..4: mod_base = OTHER; note = phosphorothioate bond
  misc_difference 25..27: note = A or T or C or G
  misc_difference 28: note = A or T
  misc_difference 29..31: note = A or T or C or G
  misc_difference 32: note = A or T
  misc_difference 33..34: note = A or T or C or G
aactacacgacgctcttccgatctnnnwnnnwnnagtgacac

SEQ ID NO: 3 - DNA, 59 bp, synthetic construct
  source 1..59: mol_type = other DNA
  modified_base 1..4: mod_base = OTHER; note = phosphorothioate bond
  misc_difference 38..39: note = A or T or C or G
  misc_difference 40: note = A or T
  misc_difference 41..42: note = A or T or C or G
  misc_difference 43: note = A or T
  misc_difference 44..45: note = A or T or C or G
  misc_difference 54..59: note = A or T or C or G
aaattgactggagttcagacgtgtgctcttccgatctnnwnnwnnagactctgnnnnnn

SEQ ID NO: 4 - DNA, 50 bp, synthetic construct
  source 1..50: mol_type = other DNA
  modified_base 47..50: mod_base = OTHER; note = phosphorothioate bond
  modified_base 1: mod_base = OTHER; note = 5' PHOSPHORYLATION
  misc_difference 9..10: note = A or T or C or G
  misc_difference 11: note = A or T
  misc_difference 12..13: note = A or T or C or G
  misc_difference 14: note = A or T
  misc_difference 15..16: note = A or T or C or G
cagagtctnnwnnwnnagatcggaagagcacacgtctgaactccagtcaa

SEQ ID NO: 5 - DNA, 12 bp, Homo sapiens
  source 1..12: mol_type = genomic DNA
  telomere 1..12
ttagggttaggg

SEQ ID NO: 6 - DNA, 12 bp, Homo sapiens
  source 1..12: mol_type = genomic DNA
  telomere 1..12
  misc_feature 1..12: note = antisense
ccctaaccctaa

SEQ ID NO: 7 - DNA, 12 bp, synthetic construct
  source 1..12: mol_type = other DNA
cgttagggatcg

SEQ ID NO: 8 - DNA, 18 bp, Homo sapiens
  source 1..18: mol_type = genomic DNA
  telomere 1..18
ttagggttagggttaggg

SEQ ID NO: 9 - DNA, 24 bp, Homo sapiens
  source 1..24: mol_type = genomic DNA
  telomere 1..24
ttagggttagggttagggttaggg

SEQ ID NO: 10 - DNA, 12 bp, synthetic construct
  source 1..12: mol_type = other DNA
cgatcgatcgat

SEQ ID NO: 11 - DNA, 12 bp, synthetic construct
  source 1..12: mol_type = other DNA
  misc_feature 1..12: note = antisense
cgccctaaatcg

SEQ ID NO: 12 - DNA, 18 bp, Homo sapiens
  source 1..18: mol_type = genomic DNA
  telomere 1..18
  misc_feature 1..18: note = antisense
ccctaaccctaaccctaa

SEQ ID NO: 13 - DNA, 24 bp, Homo sapiens
  source 1..24: mol_type = genomic DNA
  telomere 1..24
  misc_feature 1..24: note = antisense
ccctaaccctaaccctaaccctaa